SEQUENCE LISTING (35 sequences)

SEQ ID NO: 1 - DNA, 2134 bp, Oryza sativa
gaaactaccagaatgcccgtatgttgcaacacgattttaattaattaaagaaaatatcattaatctattattataatcttttaattcaattgatgtagatatttgttcctgacttgtggatttttttagtctcacactctcccttttttttctttctcatgggagtggtcgatatgtgcagcgtctaacaccacatgtcctttttcttcgaaaaaagagttctcccttataacttttataaggagtatagatagtttgcattccttgcttgctgctttttttatagatataatttgcatttccttgttgcactgatcattagctcattagttcatcccatgaggacatgatgacagtatgacactacccagaatgattatttttcacttaatatttggagatttatctcgacgtcactgggtggcatataagggatgcgacaaacactccatatcaatcgaggaatttcctctcgtcttgaattatgactgatcatgacttgtcattagagaagttaaagaaaaagatgggacttatcaggagagactaaaaaaagagtagccctacgagtagtttagtgtgttttgccatactaccatgtgatggtgatgtcaaaaacatggggctcgcgtaaccgcaaaactaaggctgtcccagacacactaatcaaaagcattcgaatttagaactcctataatttgttagtttgtttaatcactctttgtgtctcatgatgattcatgcttgcataattgcatacacttctcgactctaagcttggaccacacaacccgtacataggacgaaaccgaaagcatcacccaaaaaaaaaaaaactaatgtgcccctctttagcatgtcagaataatatggaaaaggcgtcgagcagaaacaaatatgtcaagagtttgacaaacctgacaactgccaatgcagccaatgagccaagcagcaacttgttgatggctggtaggctttcccattatattctggcaaaaactttgcttccatttttctattgacgacgatgagttcgccgattccggccgggccggctcagcttttctccttcggctagcgtttggacgcacacttttgagagaatcgaaacacggagagaagtgctccgatgcatgacacctccttttccacgtacttaacgccaaagggaaacaatgatatgcagtgaacacaaagggattgaccctgcccaggatcatcaatacacgttgaggcttcttcgtgtgtgaaagcatggctaactcgtctggtcaaaggcaaaattgctgctatcctaaaacaaatagtactaggataaataaatgaaagggctaatataacactcgcacacacgcgagacgacaaaatggagagctagcacacacgatagggacggctcgacggtaggggaggaggaataatgcaggcaggatgttactatggccacgtttagttggttagtgtgacaaaaaatttttaaaagtatacagacatacataatatatatttaaagtattaaaggtagactaataacaaaataaattacagatttcacatataaactgtgaggtgaatttattaagtttaattaatccttcattagcaaatgtttactgtagcatcacattgtcaaatcatggcgtaattagactcaaaagattcgtttcgcaatttacatgcaaactgtgcaattggtttttttccatatttaatgcttcatatatgtattcaaatatttgatgtgacggaattttttaaagtttaaattcaaatatttgatgtgacggaattttttaaagtttgaagggaaataaacacggcctatggcgcaaaattgtactacaaactccctttcgcgagacgagagaatgcaacgccgtgccgctctggcggctggggcccaaccgcgaaaggcaatccatccctctctctccccgtcgcgtaaggccgctataaatcaacccgtcgcgggttgcattgcatcggtcggtctcggtcgctgccgtccctgtgcttggagtagacacgagccggtgacgcgcatatcgtcttctgctcgagatcgatcgatcgatcgcttggttggttgctgcgactgcgagggaggccgcgggtggtgaggaggattgtgcaa

SEQ ID NO: 2 - DNA, 4061 bp, Oryza sativa
gatttggatgaacttgggttgcatttgggctttctatatgggcctgaatttgaatttccttgggatgcaaaattctctcgaggaaaaaagtacaccgaaggtccctcaacttggcatcgagttacaaaatcgtcccccaaccacaaaaccggatacaacgcatcccccaacttataaaaccagtgcatactgatcccggttttagctgacatggcggctgactcagcgtggaacccacgtgggccccatatgtcaggatgccacgtcggccagcctctcccttcctttcctgtcccctcctttcgtctcgacttgttaggatgcaacgtcatcagcagggcggccggcggtggggagggggttgccagggtgagaggtccgacggccggcgggcgatgcggcggcggcaacgtggcggctgagacacgagagaaggggaaggcagcggaccagcgttgtggcggagaggcggcagcaggcgtgcgagtgtcgtggcggagagggcggaggcggtagctgcggcggagaggcgatggcggcggatgcgagcgtcgtggcggagaggaggcggcggcggcggctgccacgcgggagggcaggccgaaggcgacggcgtgggcgagcgtggaggcacgcgacggcgaggctgccgctccgcccgtcgcctcttctctctcaggtttctcctcgtctgggtgggcaggtcggccggcgggttgggaaggaagggaaggggttggggtgatggcttgctggagctgcttcggctgcagccggtcgatcgcagcaggaggcgaggtccggctgccggagccgttccagctgccggcgcccttgcccagctagccgcatggtctagggacttgttcgctttcatcgttcttcgaattctctccccgtttcttcgtgtactgtttaattggttcttaatctgctcataaattaatcgtcgtcatctatggcctggattgccggatagtatctttgtgcttgtaaattttcagcggctatgcgagagctcgtgccaaatttgatatgcgagagttttgttgtttgtttcgccggcgaccgagcaggacgggtccgggcaccggagcgacaggcaccgcgcgccgtcgcccaccgccacgcggaaacgaccggtgatgcggcagccgcaccggcctgccggaggaccgggcaaatggtgggcgaagcgggcaacgacgcgggccggagtggccggccggcggtgggcaggcacgctgcgggagaggaaggggctgcggcggcaggggcgcagaaggtcaggccgcaccggcaggcggctgtcgacatcgaccatttagagggcagagagaggacaagaacggggagaggagagagcggaggcggatggcgccgacggggagcgggatggggatagcaaggccgtcatcgccgtcgtcgtccatggcgtggggaggccgcctccgctccgcctgtcgcctgcctgccgccgccgacccgcctgcccaccggccgccgaacctcctcctctcccgcctcgccccgtcggcttcctccccaacccgtcggccggcctgcccacccagacgaggtgaaagctgagagagaagaggggaaagagagagatgacgtggcatcctgacatgtgggttccacgctgacatgtgggttccacgctgagtcagctgctacgtcggaaaaaactgaggtcaaaaccgccgagggacctagtttgccctggttttgtaagttggaggatgcgttgtatccggttttgcggttggaggacgattttgtaactcgatgacaagttgagggaccttcggtgtactttttccttctctcgatgccactggattgggccggtttggaccaatttgcgtgcattttgagctttccatgtgggccgtagtttgcattccttttttttttgagaaactaccagaatgcccgtatgttgcaacacgattttaattaattaaagaaaatatcattaatctattattataatcttttaattcaattgatgtagatatttgttcctgacttgtggatttttttagtctcacactctcccttttttttctttctcatgggagtggtcgatatgtgcagcgtctaacaccacatgtcctttttcttcgaaaaaagagttctcccttataacttttataaggagtatagatagtttgcattccttgcttgctgctttttttatagatataatttgcatttccttgttgcactgatcattagctcattagttcatcccatgaggacatgatgacagtatgacactacccagaatgattatttttcacttaatatttggagatttatctcgacgtcactgggtggcatataagggatgcgacaaacactccatatcaatcgaggaatttcctctcgtcttgaattatgactgatcatgacttgtcattagagaagttaaagaaaaagatgggacttatcaggagagactaaaaaaagagtagccctacgagtagtttagtgtgttttgccatactaccatgtgatggtgatgtcaaaaacatggggctcgcgtaaccgcaaaactaaggctgtcccagacacactaatcaaaagcattcgaatttagaactcctataatttgttagtttgtttaatcactctttgtgtctcatgatgattcatgcttgcataattgcatacacttctcgactctaagcttggaccacacaacccgtacataggacgaaaccgaaagcatcacccaaaaaaaaaaaaactaatgtgcccctctttagcatgtcagaataatatggaaaaggcgtcgagcagaaacaaatatgtcaagagtttgacaaacctgacaactgccaatgcagccaatgagccaagcagcaacttgttgatggctggtaggctttcccattatattctggcaaaaactttgcttccatttttctattgacgacgatgagttcgccgattccggccgggccggctcagcttttctccttcggctagcgtttggacgcacacttttgagagaatcgaaacacggagagaagtgctccgatgcatgacacctccttttccacgtacttaacgccaaagggaaacaatgatatgcagtgaacacaaagggattgaccctgcccaggatcatcaatacacgttgaggcttcttcgtgtgtgaaagcatggctaactcgtctggtcaaaggcaaaattgctgctatcctaaaacaaatagtactaggataaataaatgaaagggctaatataacactcgcacacacgcgagacgacaaaatggagagctagcacacacgatagggacggctcgacggtaggggaggaggaataatgcaggcaggatgttactatggccacgtttagttggttagtgtgacaaaaaatttttaaaagtatacagacatacataatatatatttaaagtattaaaggtagactaataacaaaataaattacagatttcacatataaactgtgaggtgaatttattaagtttaattaatccttcattagcaaatgtttactgtagcatcacattgtcaaatcatggcgtaattagactcaaaagattcgtttcgcaatttacatgcaaactgtgcaattggtttttttccatatttaatgcttcatatatgtattcaaatatttgatgtgacggaattttttaaagtttaaattcaaatatttgatgtgacggaattttttaaagtttgaagggaaataaacacggcctatggcgcaaaattgtactacaaactccctttcgcgagacgagagaatgcaacgccgtgccgctctggcggctggggcccaaccgcgaaaggcaatccatccctctctctccccgtcgcgtaaggccgctataaatcaacccgtcgcgggttgcattgcatcggtcggtctcggtcgctgccgtccctgtgcttggagtagacacgagccggtgacgcgcatatcgtcttctgctcgagatcgatcgatcgatcgcttggttggttgctgcgactgcgagggaggccgcgggtggtgaggaggattgtgcaaaaaaa

SEQ ID NO: 3 - DNA, 10000 bp, Oryza sativa
gatttggatgaacttgggttgcatttgggctttctatatgggcctgaatttgaatttccttgggatgcaaaattctctcgaggaaaaaagtacaccgaaggtccctcaacttggcatcgagttacaaaatcgtcccccaaccacaaaaccggatacaacgcatcccccaacttataaaaccagtgcatactgatcccggttttagctgacatggcggctgactcagcgtggaacccacgtgggccccatatgtcaggatgccacgtcggccagcctctcccttcctttcctgtcccctcctttcgtctcgacttgttaggatgcaacgtcatcagcagggcggccggcggtggggagggggttgccagggtgagaggtccgacggccggcgggcgatgcggcggcggcaacgtggcggctgagacacgagagaaggggaaggcagcggaccagcgttgtggcggagaggcggcagcaggcgtgcgagtgtcgtggcggagagggcggaggcggtagctgcggcggagaggcgatggcggcggatgcgagcgtcgtggcggagaggaggcggcggcggcggctgccacgcgggagggcaggccgaaggcgacggcgtgggcgagcgtggaggcacgcgacggcgaggctgccgctccgcccgtcgcctcttctctctcaggtttctcctcgtctgggtgggcaggtcggccggcgggttgggaaggaagggaaggggttggggtgatggcttgctggagctgcttcggctgcagccggtcgatcgcagcaggaggcgaggtccggctgccggagccgttccagctgccggcgcccttgcccagctagccgcatggtctagggacttgttcgctttcatcgttcttcgaattctctccccgtttcttcgtgtactgtttaattggttcttaatctgctcataaattaatcgtcgtcatctatggcctggattgccggatagtatctttgtgcttgtaaattttcagcggctatgcgagagctcgtgccaaatttgatatgcgagagttttgttgtttgtttcgccggcgaccgagcaggacgggtccgggcaccggagcgacaggcaccgcgcgccgtcgcccaccgccacgcggaaacgaccggtgatgcggcagccgcaccggcctgccggaggaccgggcaaatggtgggcgaagcgggcaacgacgcgggccggagtggccggccggcggtgggcaggcacgctgcgggagaggaaggggctgcggcggcaggggcgcagaaggtcaggccgcaccggcaggcggctgtcgacatcgaccatttagagggcagagagaggacaagaacggggagaggagagagcggaggcggatggcgccgacggggagcgggatggggatagcaaggccgtcatcgccgtcgtcgtccatggcgtggggaggccgcctccgctccgcctgtcgcctgcctgccgccgccgacccgcctgcccaccggccgccgaacctcctcctctcccgcctcgccccgtcggcttcctccccaacccgtcggccggcctgcccacccagacgaggtgaaagctgagagagaagaggggaaagagagagatgacgtggcatcctgacatgtgggttccacgctgacatgtgggttccacgctgagtcagctgctacgtcggaaaaaactgaggtcaaaaccgccgagggacctagtttgccctggttttgtaagttggaggatgcgttgtatccggttttgcggttggaggacgattttgtaactcgatgacaagttgagggaccttcggtgtactttttccttctctcgatgccactggattgggccggtttggaccaatttgcgtgcattttgagctttccatgtgggccgtagtttgcattccttttttttttgagaaactaccagaatgcccgtatgttgcaacacgattttaattaattaaagaaaatatcattaatctattattataatcttttaattcaattgatgtagatatttgttcctgacttgtggatttttttagtctcacactctcccttttttttctttctcatgggagtggtcgatatgtgcagcgtctaacaccacatgtcctttttcttcgaaaaaagagttctcccttataacttttataaggagtatagatagtttgcattccttgcttgctgctttttttatagatataatttgcatttccttgttgcactgatcattagctcattagttcatcccatgaggacatgatgacagtatgacactacccagaatgattatttttcacttaatatttggagatttatctcgacgtcactgggtggcatataagggatgcgacaaacactccatatcaatcgaggaatttcctctcgtcttgaattatgactgatcatgacttgtcattagagaagttaaagaaaaagatgggacttatcaggagagactaaaaaaagagtagccctacgagtagtttagtgtgttttgccatactaccatgtgatggtgatgtcaaaaacatggggctcgcgtaaccgcaaaactaaggctgtcccagacacactaatcaaaagcattcgaatttagaactcctataatttgttagtttgtttaatcactctttgtgtctcatgatgattcatgcttgcataattgcatacacttctcgactctaagcttggaccacacaacccgtacataggacgaaaccgaaagcatcacccaaaaaaaaaaaaactaatgtgcccctctttagcatgtcagaataatatggaaaaggcgtcgagcagaaacaaatatgtcaagagtttgacaaacctgacaactgccaatgcagccaatgagccaagcagcaacttgttgatggctggtaggctttcccattatattctggcaaaaactttgcttccatttttctattgacgacgatgagttcgccgattccggccgggccggctcagcttttctccttcggctagcgtttggacgcacacttttgagagaatcgaaacacggagagaagtgctccgatgcatgacacctccttttccacgtacttaacgccaaagggaaacaatgatatgcagtgaacacaaagggattgaccctgcccaggatcatcaatacacgttgaggcttcttcgtgtgtgaaagcatggctaactcgtctggtcaaaggcaaaattgctgctatcctaaaacaaatagtactaggataaataaatgaaagggctaatataacactcgcacacacgcgagacgacaaaatggagagctagcacacacgatagggacggctcgacggtaggggaggaggaataatgcaggcaggatgttactatggccacgtttagttggttagtgtgacaaaaaatttttaaaagtatacagacatacataatatatatttaaagtattaaaggtagactaataacaaaataaattacagatttcacatataaactgtgaggtgaatttattaagtttaattaatccttcattagcaaatgtttactgtagcatcacattgtcaaatcatggcgtaattagactcaaaagattcgtttcgcaatttacatgcaaactgtgcaattggtttttttccatatttaatgcttcatatatgtattcaaatatttgatgtgacggaattttttaaagtttaaattcaaatatttgatgtgacggaattttttaaagtttgaagggaaataaacacggcctatggcgcaaaattgtactacaaactccctttcgcgagacgagagaatgcaacgccgtgccgctctggcggctggggcccaaccgcgaaaggcaatccatccctctctctccccgtcgcgtaaggccgctataaatcaacccgtcgcgggttgcattgcatcggtcggtctcggtcgctgccgtccctgtgcttggagtagacacgagccggtgacgcgcatatcgtcttctgctcgagatcgatcgatcgatcgcttggttggttgctgcgactgcgagggaggccgcgggtggtgaggaggattgtgcaaaaaaaatggggcgcgcgccgtgctgcgacaaggcgagcgtgaagcgggggccgtggtcgccggaggaggacgagcagctgcggagctacgtccagagccacggcatcggcggcaactggatcgcgctcccgcagaaagcaggtcggtattagtgcactgctcgtcgtcgtcgatgctagccggtgatgaaacgccatgtttgtatgtgaattctgatgtctgcgtgttctcttgtgtgattcagggctcaaccgctgcggcaagagctgcaggctccggtggctcaactacctgaggccggacatcaagcacggcggctacaccgagcaggaggaccacatcatctgctcgctctacaactcgattggaagcaggtgatgatttaagccggagatgaatatttcttttttctttcactgatcctgttctgagttcttgaactgactctgtttgcgtgcgttgccaggtggtccatcatcgcgtcgaagctccccggccggactgacaacgacgtcaagaactactggaacaccaagctcaagaagaaagccatgggcgccgtgcagccgcgcgccgccgcctcggcgccgagccaatgcacgtcgtcagcgatggcgccggcgctctcgccggcctcctcgtccgtcaccagctcgagcggcgacgcctgcttcgccgccgccgccaccaccaccaccaccatgtacccgccaccgacgacgccgccgcagcagcagttcatccgcttcgacgcaccacccgcggcggcggcggcggcatccccgaccgacctcgcgcccgtgccaccaccggccaccgtcacggcggacggcgacggcggctgggcgtccgacgccttgtccctcgacgacgtgttcctcggcgagctcacggccggcgagccgctgttcccttacgccgagctgttcagcggcttcgccggcgcggcgccggacagcaaggccaccttggagctctcggcgtgctacttcccgaacatggcggagatgtgggcggcctccgaccacgcctacgccaagccacagggtctctgcaacaccctgacataggaggacacgctaaaacctccattgaattcttgcgtagctgcgaacgcaacggcgatcgatcgatgcgtcgccacttgctactagcataattcttgctctcttttttttttcaatttttttctcttcttcgagtggcccattctctagctggcgttttctcgtgtattatgagtgatcgattattacatgtaaattaatccatagatgtatctgcactgatccaaattctccagcgattacgcttgtcagttcgtacgcgatccatccgttggcttggctctccggtacaccggacaagttagcgcgtcccgaaaacgatacgcccctgtctgtctccctctcgccgagccgaagtcacttcacatgtacagtgtcacagtgacaaatctgacgagacgaaatgttaggtgaggcacaagaggttcagaagaacaacgtgtgcgagacatggaccagttctcgtgcggcgcgcacgcacagtgtcatggacttgacccgttccggttttcgagggccaatggggaggagaggcgtgtggacggttttctgtcacgttacacgtaggagtattttttttcctctcattttgtttttgatttttgcccggtggcaagttgatcctgatcgtatggcctagggggcctatgggccgtggatcacatggtaattgggtgtcattgtttatttgggtagtctcacttggggcatggatagtgtctctttgtaaagtgattgaagacttcaagtgcacgacaaggacgagcctagttgaccctggaaatggagggaggatcggtttcgaagaagcttccttgacaattgtcatgctcgtgatgggctctatacatatgatcagaggagacctctagttaaaagtctctctaactaattactccaaaaaaaagtctctctaactttgctcattgtgggtttatcaatgtctaggcaaattaaaaccatcaataataacatgttaacagtacttaccttgttggattttactagcttagtgtacgccagggatccacacattttccaggtcttcgtggtcggcatgagaaaatccggcaaaagccaagtagaacaaggccaaaggatgggaaaaagccagtgcaatcctccactgtagttttatttggaaagtagatgaatatggattttttttataaaaaacaaacttttatttacatttgaaaacaaaccccaaaataacttattttacaaataaaccatttgtggagaaaacaacaggcaaggtccccttataacgttgcattttttcattttggtcttttaagacaattgagtcgaaaaatcctatataattcaacctaggttataagcggcatctactacacaacggtaatggcagaggattcacttccaaggtgtacatctatctggtttgtcaagcaaatctttaatttcgttgcttgacattgtcttgcgagggatcaagtttcagttttggttaaaaatatggcaccacattttttttacttgttcttaatcactcttcccctatgcgattgttacttttaaccatagttctctttatatagcgtgtattacccgtcaacaacgaggcgtctgtggtgactttgtcaatctccaagatttaccggtccagccttcaaagatgctcataggggtagggtttgtgtgcgtgcgttcataagggtgagggtgcgtgtgtgttgtgagtgtctgcgttgtattgtgtagttctaaaaaaaacgtaattataaaaagacagtgcaattacgtagtaaatataggtaatttattgtgtaaaaacgatttgataatatattttgaaaaatgtggtatgataaatatagctagttccattaagttttagtccatgtttaacatatatttgtacctcgttcgtgtttccttcatacgcatagcagtaacttatgttttgcattttacaaagcagttacgatgagtaggtcattgtagctcatttacatatggagggttgtgattttgttagtaccttgacattgactcaaagcggcacatctcggcatcaaaggtaggtggcaaaatgcattcgtcaaggacaaatagacaaacatgcattgatcgaatagtgccttcagagccgtggacttctgcgttatcagttcgttgaggtatctctttgatttgacatgagaaatggaccatgtgacagcaagaacgaaatgcatggatgcagtgtcctaactggatagcttgctcctttcagaattaaaagctgctcacttgctagctcagataagtgtcaagacatagtaacagtgtcttcagactctgctttggtttgttgctctaactgaagaattattgctcaccaaacggataagcatatgcacatgtatcagcaaccggcattcggttggaccagtactgacgcagattgcaaacattgttaactcctcgcgatgtctaactgtcttgaaaacgtccttagaaatacttataccggtcaagatcacgagtgtattccaaataattaatttcacctacatcgatgacagaggtgtcaacttaaacatccatagcttcacaatcaagacttcaaatgttttgatgcactagaaagcaaaatcagtaacctttactatgatcatatatcacacacgaatggtgcagctgttagtgtaacaccacgtgttgagatttgagaaagcaggcccaaatcaatgcattattgtagctccatttcactatatcaagtaataatagtaatttatatatatcgtagcagcgaacatgtaccattagattaaaatatgaaaggctcagattcattgcagttctttagtgtgcacttgcaatatcgaagtgcaccctgatctcaatccatcacacatgcacacagactgttcatgtcatcgctgatgctgagttggtcgtgaaatcggacgattccacgacggcaagtacagacacacgtcgattgccggcaaccgcaagcaagtgcagtgcagggtgcaagtgcagcagggcccttggaaacgaagtcggagagagaccatccgcccatgccatgtccatgtccgtgtgtgtgcgctcgctgctgttgtctctttctgagctggtactagtaataaaaactagcccgaggattattgcaagaacaaaaggtaagcagcatcttgcagagcatactatcacccaagctcttaaagctgtcggggaaaagggaccctacgttactcagatacatacggatacgcgatacgtcgatacggggatacgacattttccggatacgacattttccaaaaaaaacaaggatacggggatacgtatatatatatatatatatatattgaaaacacacaaaaaaaattaaggaaattacattaatatatattaaaaatacaaaaaaaataaggagcttttataaagaaaaagcgatataatactagtattagtgtagccgattccatatcaatattttgcaattttcttttctataagcaattcaaattaacaagcgattaagatcccaaaaactacatattaatagttcaatcatataactcttgatgactttgcatcaacattagcagccgcactaaccagtaaccactagaattgctagatgcactagcacttgtatctaacaaaaacgatagtactttgtcctaagaaaaacaagaaaggcccaacagattcaagcaagaaatgttcaaagagcatggatttacccggccaggcactcaagcaaacaatgaaaaagctccttgctttgatgttgccagcggccagcctgccaccggcctccgctatgcccggcaacggtcgtggcctccgaggttggcggcgaaggagatgcgccaccgcccagccgcggcagctcttccgtctcgcggagaggacgttgtggcccgcgcacggcaaccaagcgcgctgctgcagccgcgtctctggtggcatcgccgggcacgcacgagagacagccatcaggatgggagcgtatgaggcggcgcatccctgatgcgagcgaagacggaggcacgtcgcggccgtatcgctggcatatctcgatttattttgtttttttaaatcgcgaatatgtgggatacgtacggaatatgtatcggaacatatccgatatgtatccgtatccgttgccgtatcagatacggatacgtcgctctgtagccgtaccaggataacgttgagttagggacctaccttcagaaaatatatattcatggatgctagtacaatccagagtagtgacttcaaaagctgaagatctgagctttttgtaccgctaacaaactcagttttttttaaggctgtgtttagtttctgagattagggagaagtttggggaaagttggtaaattagaaaaaaagttgggagtttatttaagtatgaaagttttgaatgtgatgtgatggaaagttgagagtttgggggaagtttggtgtgaactaaatagggcctaatttgttttagcatcccgtgaaacatttttgctatttctataggccttgtttagttccaaactttttcttcaaactttcaacttttccatcacatcgttccaatttcaaccaaacttctaattttggcgtgaaccaaacttctcaattttctgtgtaacttttggtctcggttattggatatcctgatgcatcatgcattgactgtacatgtcataggtcttctattcattactagtacaatatacaggttttttttgggataaacttggatgctttgttcagcatctgccttttctgtcttgactcttggtaccgcttgtgaacatgtcgacatgttgttcctgctgtttttttttccttttcattttgaggcatgcgtcactaacttatgttcgagtccatttttcagcatt

SEQ ID NO: 4 - DNA, 870 bp, Oryza sativa
atggggcgcgcgccgtgctgcgacaaggcgagcgtgaagcgggggccgtggtcgccggaggaggacgagcagctgcggagctacgtccagagccacggcatcggcggcaactggatcgcgctcccgcagaaagcagggctcaaccgctgcggcaagagctgcaggctccggtggctcaactacctgaggccggacatcaagcacggcggctacaccgagcaggaggaccacatcatctgctcgctctacaactcgattggaagcaggtggtccatcatcgcgtcgaagctccccggccggactgacaacgacgtcaagaactactggaacaccaagctcaagaagaaagccatgggcgccgtgcagccgcgcgccgccgcctcggcgccgagccaatgcacgtcgtcagcgatggcgccggcgctctcgccggcctcctcgtccgtcaccagctcgagcggcgacgcctgcttcgccgccgccgccaccaccaccaccaccatgtacccgccaccgacgacgccgccgcagcagcagttcatccgcttcgacgcaccacccgcggcggcggcggcggcatccccgaccgacctcgcgcccgtgccaccaccggccaccgtcacggcggacggcgacggcggctgggcgtccgacgccttgtccctcgacgacgtgttcctcggcgagctcacggccggcgagccgctgttcccttacgccgagctgttcagcggcttcgccggcgcggcgccggacagcaaggccaccttggagctctcggcgtgctacttcccgaacatggcggagatgtgggcggcctccgaccacgcctacgccaagccacagggtctctgcaacaccctgacatag

SEQ ID NO: 5 - protein, 289 aa, Oryza sativa
MGRAPCCDKASVKRGPWSPEEDEQLRSYVQSHGIGGNWIALPQKAGLNRCGKSCRLRWLNYLRPDIKHGGYTEQEDHIICSLYNSIGSRWSIIASKLPGRTDNDVKNYWNTKLKKKAMGAVQPRAAASAPSQCTSSAMAPALSPASSSVTSSSGDACFAAAATTTTTMYPPPTTPPQQQFIRFDAPPAAAAAASPTDLAPVPPPATVTADGDGGWASDALSLDDVFLGELTAGEPLFPYAELFSGFAGAAPDSKATLELSACYFPNMAEMWAASDHAYAKPQGLCNTLT

SEQ ID NO: 6 - protein, 289 aa, Sorghum bicolor
MGRAPCCDKASVKRGPWSPEEDEQLRSYVQRNGIGGNWIALPQKAGLNRCGKSCRLRWLNYLRPDIKHGGYTEEEDRIIWSLYSSIGSRWSIIASKLPGRTDNDVKNYWNTKLKKKAMAMAAAAGLSASSSGGGAFAAAAAPATPTPPALSPASSSVTSSSGDVRFGAAYTEPQPQPQHPGGLIRFDAPRTELAPVPPAVGAQLDGAWSTPVAPALDGGDVFLPELIGGGEQLFPYGGDFFGGLLQDSRALEQLSACYFPNMAEIWGATAAVAAPDGNCKPPGLCNTLT

SEQ ID NO: 7 - protein, 262 aa, Zea mays
MGRAPCCDKASVKRGPWSPEEDEQLRSYVQLNGIGGNWIALPQKAGLNRCGKSCRLRWLNYLRPNIKHGGYTDEEDRIIWSLYSSIGSRWSIIASKLPGRTDNDVKNYWNTKLKKKAMAASSSGAAFAAPATPALSPASSCVTSSSSGDVRLGAAYTEPPPPRQHAGLVRSDAPRTELAPVPAVAHLDGACWPAAAALDVLLPELGGEQLFPYGYGDFFGGLQDRALEQQLSSCYFPNVAEIWGAAAVAPDGKPPGLCNRLT

SEQ ID NO: 8 - protein, 259 aa, Vitis vinifera
MGRAPCCDKANVKRGPWSPEEDATLRNYVQTHGIGGNWIALPRKAGLRRCGKSCRLRWLNYLRPDIKHGGFTEEEDKVICAIYNQMGSRWSVIASQLPGRTDNDVKNYWNTKLKKKLAKFHSNGNAANNPNPFSSAPPPTAAPMPVTEAYRHGFPPLLTQGYTGTSNNMGVSEFGASSNNGHNVFISDQGGLSTDHATGISYVSYLPGTRGGEDDGVLMDFGMGNLPYDIISGFWFQEKDSTEVTPGHDQSRFLLIDGN

SEQ ID NO: 9 - protein, 302 aa, Populus trichocarps
MGRAPCCDKANVKRGPWSPEEDATLKSYLETHGTGGNWIALPQKAGLKRCGKSCRLRWLNYLRPDIKHGGFTEEEDNIICTLYSQMGSRWSLIAAQLPGRTDNDVKNYWNTKLKKKILAGKISLTIKNNPINAPVNVANIPAIPCSTSPILYVPKAETESSVTFSDHYSLTQISGTLPALSDIGYEPIINSTAQNLSPNQFQFSSFPGVIDMSEFSMNSTHIVSPSQEGSSISDSSSLAMDNKGLSVPSTNGGLEDVGIFMDSEPGFPSDFFNGLLLQDKASEVATNCYQYFAGFGYVDIKP

SEQ ID NO: 10 - protein, 348 aa, Malus x domestica
MGRAPCCDKANVKKGPWSPEEDSKLKEYIEKYGTGGNWIALPQKAGLKRCGKSCRLRWLNYLRPNIKHGEFSDEEDRIICSLFASIGSRWSVIAAQLPGRTDNDIKNYWNTKLKKKLMGMHMGPPRPHNHHKNLLKPPPFPSSSHHNYQNQPLIPSEPLSSLYKDLSNYNRSFLGFEAPVPLPPQVSLTSNNFSNISTNSSIFQTLNYPAGVKENNNNNTLLVFGSEGSCSTSSDGSCNNQISYDYCSRSEINNINQEEMGFDHQGFMMLNYGNQWTERPNGFYFGSENNTLEFTDLDEDVKQQLISTRSKNNYNNNTIINESSKSNSFLFNVNESKTEDDEKVMYFY

SEQ ID NO: 11 - protein, 193 aa, Glycine max
MGRAPCCDKANVKKGPWSPEEDAKLKSYIEQHGTGGNWIALPQKIGLKRCGKSCRLKWLNYLRPNIKHGGFSEEEDNIICSLYVSIGSRWSIIAAQLPGRTDNDIKNYWNTRLKKKLLGKQRKEQQAQARRVSNNQQKQDVKRETENLIVASEVMINQVPYWSSSEFSSVPMPVTNASSFQHYGLNNQTSLRA

SEQ ID NO: 12 - protein, 313 aa, Daucus carota
MGRAPCCDKASVKRGPWSPEEDQKLKDYIEKHGTGGNWIALPQKAGLRRCGKSCRLRWLNYLRPNIKHGEFSDDEDRIICTLFANIGSRWSIIAGQLPGRTDNDIKNYWNTKLKKKLLMSSLMLPNPFVRPPNNINNYQQYLLSSPSPSSYSSPLPPTLRLCDNNYPTLGASRSFTSEGFSNISTNLFNIQPQDQGLGPMQNYQEKESPLIMFGGAGDDQQAASSSSYDEHFMMFSNCGMNNISDQQKQVNLGVSSGDHEIVSNVLDQYNMSCVEEIKQLISTTNLSCNSNLSFFVDENKTVRSEEEKVLMYY

SEQ ID NO: 13 - protein, 333 aa, Arabidopsis thaliana
MGRAPCCDKANVKKGPWSPEEDVKLKDYIDKYGTGGNWIALPQKIGLKRCGKSCRLRWLNYLRPNIKHGGFSEEEDRIILSLYISIGSRWSIIAAQLPGRTDNDIKNYWNTKLKKKLLGRQKQMNRQDSITDSTENNLSNNNNNKSPQNLSNSALERLQLHMQLQNLQSPFSSFYNNPILWPKLHPLLQSTTTNQNPKLASQESFHPLGVNVDHQHNNTKLAQINNGASSLYSENVEQSQNPAHEFQPNFGFSQDLRLDNHNMDFMNRGVSKELFQVGNEFELTNGSSWWSEEVELERKTTSSSSWGSASVLDQTTEGMVMLQDYAQMSYHSV

SEQ ID NO: 14 - DNA, 870 bp, Sorghum bicolor
atggggcgcgcgccgtgctgcgacaaggcgagcgtgaagcggggaccctggtcgcctgaggaggacgagcagctgcggagctacgtccagcgcaacggcatcggcggcaactggatcgccctgccgcagaaagcaggcctgaaccggtgcggcaagagctgccggctgcggtggctcaactacctgcggccggacatcaagcacgggggctacaccgaggaggaggaccggatcatctggtcgctctacagctccatcggcagcaggtggtccatcatcgcgtccaagctcccgggccggaccgacaacgacgtcaagaactactggaacaccaagctcaagaagaaggccatggccatggcggcggcggccggcctcagcgccagtagcagcggaggaggagccttcgcggcggcggcggcgcccgccacgccgacgccgccggcgctctcgcccgcctcctcgtccgtcaccagctccagcggcgacgtgcgcttcggcgcggcgtacacggaaccacaaccgcagccgcagcaccccggcgggctcatacgcttcgacgcgccgcggacggagctcgcgcccgtgccgccggcagtgggagcgcagctggacggcgcctggtcgacgcccgtggcgccggcgctggacggcggggacgtgttcctgcccgagctcatcggcggcggcgagcagctgttcccgtacggcggcgacttcttcggcgggctgctgcaggacagcagggccctggagcagctctcggcgtgctacttccccaacatggccgagatatggggcgccaccgctgccgtcgccgcccccgacggcaactgcaagccgccgggtctctgcaacaccctgacatag

SEQ ID NO: 15 - DNA, 789 bp, Zea mays
atgggccgcgcgccgtgctgcgacaaggcgagcgtgaagcgggggccctggtcgcccgaggaggacgagcagctgcggagctacgtccagctgaacggcatcggcggcaactggatcgccctgccgcagaaagcagggctgaaccggtgtggcaagagttgccggctgcggtggctcaactacctgcggccgaacatcaagcacggcggctacaccgacgaggaggaccggatcatttggtcgctctacagctccatcggcagcaggtggtcgatcatcgcgtccaagctccccggccggaccgataacgacgtcaagaactactggaacaccaagctcaagaagaaggccatggcggccagtagcagcggagcagccttcgcggcgcccgccacgccggcgctctcgcccgcctcctcgtgcgtcaccagctcctccagcggcgacgtgcgcctcggcgcagcgtacacggagccgccgccgcctcggcagcacgcggggctcgtacgctctgacgctccgcggacggagctcgcgcccgtgccggcagtcgcgcacctggacggcgcctgctggccggccgcggcggcgctggacgtgctcctgcccgagctcggcggcgagcagctgttcccgtacgggtacggcgacttcttcggcgggctgcaggacagggccctggagcagcagctctcgtcgtgctacttccccaacgtggccgagatatggggcgccgctgccgtcgcccccgacggcaagccgccgggtctctgcaacaggctgacgtag

SEQ ID NO: 16 - DNA, 909 bp, Populus trichocarps
atggggagagctccttgttgtgacaaagcaaacgtgaaaagaggaccatggtctcctgaggaagatgccactctcaaaagctaccttgagactcatggcactggtggtaactggattgctttgcctcaaaaagctggccttaagcgatgcggcaagagctgccggttacgatggcttaattatctcagaccagacatcaaacatggaggctttactgaagaagaagacaacatcatatgcactctctatagtcaaatgggaagcagatggtctctcatagctgctcaactgcctggaagaactgacaacgatgtgaaaaattactggaacaccaaattgaaaaagaagattcttgcgggaaaaataagcctcacaatcaagaacaaccctatcaatgcccccgttaacgttgctaatattcctgccattccctgctcaacatctcctattctttatgtccctaaagctgaaaccgaaagttctgtcaccttctccgatcattattcattaactcaaatttcaggaacattgcctgccttatctgatattggttatgaacctattatcaatagcactgcccagaacttgagtccaaaccaattccaattttctagtttccctggggtcattgacatgtctgaattttcaatgaacagtactcacattgtttctccatctcaagaaggttcgagcatttcagattcctcttcacttgctatggataacaagggtctctcagtgcctagtactaacggtggtcttgaggatgttgggatatttatggattccgaacctggttttcctagtgattttttcaatggccttttgcttcaagacaaagctagtgaagtggccactaattgctaccaatattttgctggttttggctatgttgatattaagccgtaa

SEQ ID NO: 17 - DNA, 1047 bp, Malus x domestica
atggggagggctccttgttgtgacaaagcaaacgtgaagaagggaccatggtcaccagaagaagattcaaagctaaaagagtacatagagaagtatgggactggtgggaattggattgctctcccacagaaagctggtctgaagagatgtgggaaaagctgcagattgagatggcttaactatctgaggccaaacatcaaacatggagaattctctgatgaggaagacaggataatatgcagcctctttgctagtattggaagcaggtggtcagttatagctgctcagctgccaggcaggactgacaacgatatcaagaactactggaacaccaagctcaagaagaagctcatggggatgcatatgggtcctcctcgacctcacaaccaccataaaaatttactaaagcctcccccatttccttcttcctctcatcacaattaccaaaaccaaccattaattccatctgaacctctatcgtcgctgtacaaagacctaagcaattacaacagatctttcttagggtttgaagcgccagtgccattgccaccacaagtttcgctgacgtccaataatttctcaaacatttccaccaactcttctatttttcaaaccctaaattacccagctggagtgaaggaaaataacaataataataccctcctcgtgtttggaagtgaagggagttgcagtacttcgtctgatggaagctgtaataatcagatcagctatgactactgcagcagatcagagattaataacatcaaccaagaagaaatgggttttgatcatcagggcttcatgatgcttaactatggcaaccaatggaccgaaaggccaaatgggttttattttggatcagaaaacaacacattagaatttactgatctggacgaagatgttaagcagcagctgattagtactagaagtaaaaataattataataataatactattataaatgagtcctctaagtctaacagctttttattcaatgttaatgaaagcaagacagaagatgatgagaaggtcatgtatttctactga

SEQ ID NO: 18 - DNA, 582 bp, Glycine max
atgggaagagctccttgctgtgacaaagccaacgtgaagaaaggtccatggtcacctgaagaagatgccaaactcaagtcttatattgagcagcatggcactggtggaaactggattgccttgccccagaaaataggccttaagaggtgtgggaaaagctgtcgtcttaaatggctgaattatcttcgcccaaacatcaaacatgggggtttctcagaagaagaagacaacatcatctgcagcctctatgttagtattggaagcaggtggtcaattatagcagcacagttacccgggcgcacagacaatgatataaagaactattggaacactaggctgaagaagaagctccttggcaaacaaaggaaggagcaacaggctcaagctcgccgagtcagcaacaaccagcagaagcaagatgtcaaaagagaaaccgagaatttgatagtggcttctgaagtcatgatcaatcaagttccctattggtcttcttcagagttttcttctgtgcctatgccagtgaccaatgcttcttcatttcaacactatggtctgaacaatcaaacatctctcagagcatga

SEQ ID NO: 19 - DNA, 942 bp, Daucus carota
atggggagagctccttgctgtgacaaggcaagtgtaaagagagggccatggtcacctgaagaagatcagaagctaaaagactatatagagaagcatgggactggaggaaactggattgctctaccacaaaaggctggtcttagaagatgtgggaaaagctgcagattaagatggctcaactatctcaggcctaacattaaacatggagaattttcagatgatgaagataggatcatatgcaccctctttgccaatattgggagcaggtggtcaataatagcaggtcagttaccagggaggactgacaatgatatcaagaactactggaacaccaagctcaagaagaaactcctcatgtcttcattaatgctccctaatccttttgttaggcctcctaataatattaataactaccaacaatacctattatcatcaccatctccttcttcatattcctccccattgcctccaacattgagactttgtgacaacaattacccaaccctaggtgcttctaggtcattcacaagtgagggcttctcaaatatttcaacaaacctctttaatatccagcctcaagatcagggtttgggtcccatgcaaaattatcaagagaaagaatcacctctcattatgtttggaggggcaggtgatgatcagcaagctgctagctctagttcttatgatgaacatttcatgatgttctccaactgtgggatgaataatatttctgatcaacagaagcaagtaaatcttggtgtgtctagtggtgatcatgagattgttagcaatgtactggatcagtataacatgagctgtgttgaggagattaagcaactgattagcactactaatctgtcatgtaattctaatctgagtttctttgttgatgaaaacaagacagtgagaagtgaggaggagaaagtgttgatgtactactga

SEQ ID NO: 20 - DNA, 1002 bp, Arabidopsis thaliana
atgggaagagctccatgctgcgacaaggcaaacgtgaagaaaggaccatggtcaccggaagaagatgtgaagctcaaggattacatcgacaaatatggcactggtggcaactggatcgcactgcctcagaaaattgggctgaagagatgtggtaagagttgcagactgagatggcttaattacttaagaccaaacatcaaacatggtggtttttctgaggaagaagatagaatcatcttgagtctctacattagcattggaagccggtggtccataattgcagctcagcttcctggaaggactgacaatgatatcaagaattattggaacacaaaactgaagaagaaacttctaggaagacagaaacaaatgaatcgtcaagactccataaccgattctactgagaacaacctcagcaacaataacaacaataagagtcctcagaatctgagcaattcggcactggagaggctccagcttcacatgcagcttcagaatctacagagccctttctctagtttctacaacaaccctatcttgtggcccaagcttcatccattgctccagagcactacaactaatcaaaaccctaagcttgcatctcaagaaagcttccaccctttaggagttaacgttgatcatcagcacaacaataccaagctagctcagataaacaatggagcctcttctctctattcggagaacgtagagcaatcccaaaaccctgctcatgaatttcaacctaatttcggtttttcacaggaccttcgattagataatcataacatggactttatgaacagaggggtttctaaagaactgtttcaagtgggcaacgagtttgagctaacgaacggttcgagttggtggtcagaggaagtggaactagagaggaaaacgacgtcgtcgagttcttgggggtcagcttctgtgcttgatcagacaactgagggaatggttatgcttcaagattacgctcagatgagctaccacagtgtttaa

SEQ ID NO: 21 - DNA, 29 bp, Artificial
  PCR primer
tggtgaggaggattgtgcaaggatccgcg

SEQ ID NO: 22 - DNA, 29 bp, Artificial
  PCR primer
ccggaattcttgcacaatcctcctcacca

SEQ ID NO: 23 - DNA, 21 bp, Artificial
  PCR primer
cgtcaagaactactggaacac

SEQ ID NO: 24 - DNA, 20 bp, Artificial
  PCR primer
ccatgttcgggaagtagcac

SEQ ID NO: 25 - DNA, 25 bp, Artificial
  PCR primer
cgcggatccatggggcgcgcgccgt

SEQ ID NO: 26 - DNA, 34 bp, Artificial
  PCR primer
cccaagctttgtcagggtgttgcagagaccctgt

SEQ ID NO: 27 - DNA, 26 bp, Artificial
  PCR primer
cacctgcggtgaatggaagacgtttg

SEQ ID NO: 28 - DNA, 28 bp, Artificial
  PCR primer
tgctcaaagcagaagagatctgaatgag

SEQ ID NO: 29 - DNA, 29 bp, Artificial
  PCR primer
caccgacggaggaagtagtgtggaaccat

SEQ ID NO: 30 - DNA, 17 bp, Artificial
  PCR primer
tggtggtagggtgcggc

SEQ ID NO: 31 - DNA, 27 bp, Artificial
  PCR primer
cacccagatcaaatgtcaaaaggggcg

SEQ ID NO: 32 - DNA, 22 bp, Artificial
  PCR primer
cttgcctgccgagctatcaacc

SEQ ID NO: 33 - DNA, 17 bp, Artificial
  PCR primer
atggggcgcgcgccgtg

SEQ ID NO: 34 - DNA, 24 bp, Artificial
  PCR primer
ctatgtcagggtgttgcagagacc

SEQ ID NO: 35 - DNA, 2144 bp, Oryza sativa
gagaaactaccagaatgcccgtatgttgcaacacgattttaattaattaaagaaaatatcattaatctattattataatcttttaattcaattgatgtagatatttgttcctgacttgtggatttttttagtctcacactctcccttttttttctttctcatgggagtggtcgatatgtgcagcgtctaacaccacatgtcctttttcttcgaaaaaagagttctcccttataacttttataaggagtatagatagtttgcattccttgcttgctgctttttttatagatataatttgcatttccttgttgcactgatcattagctcattagttcatcccatgaggacatgatgacagtatgacactacccagaatgattatttttcacttaatatttggagatttatctcgacgtcactgggtggcatataagggatgcgacaaacactccatatcaatcgaggaatttcctctcgtcttgaattatgactgatcatgacttgtcattagagaagttaaagaaaaagatgggacttatcaggagagactaaaaaaagagtagccctacgagtagtttagtgtgttttgccatactaccatgtgatggtgatgtcaaaaacatggggctcgcgtaaccgcaaaactaaggctgtcccagacacactaatcaaaagcattcgaatttagaactcctataatttgttagtttgtttaatcactctttgtgtctcatgatgattcatgcttgcataattgcatacacttctcgactctaagcttggaccacacaacccgtacataggacgaaaccgaaagcatcacccaaaaaaaaaaaaactaatgtgcccctctttagcatgtcagaataatatggaaaaggcgtcgagcagaaacaaatatgtcaagagtttgacaaacctgacaactgccaatgcagccaatgagccaagcagcaacttgttgatggctggtaggctttcccattatattctggcaaaaactttgcttccatttttctattgacgacgatgagttcgccgattccggccgggccggctcagcttttctccttcggctagcgtttggacgcacacttttgagagaatcgaaacacggagagaagtgctccgatgcatgacacctccttttccacgtacttaacgccaaagggaaacaatgatatgcagtgaacacaaagggattgaccctgcccaggatcatcaatacacgttgaggcttcttcgtgtgtgaaagcatggctaactcgtctggtcaaaggcaaaattgctgctatcctaaaacaaatagtactaggataaataaatgaaagggctaatataacactcgcacacacgcgagacgacaaaatggagagctagcacacacgatagggacggctcgacggtaggggaggaggaataatgcaggcaggatgttactatggccacgtttagttggttagtgtgacaaaaaatttttaaaagtatacagacatacataatatatatttaaagtattaaaggtagactaataacaaaataaattacagatttcacatataaactgtgaggtgaatttattaagtttaattaatccttcattagcaaatgtttactgtagcatcacattgtcaaatcatggcgtaattagactcaaaagattcgtttcgcaatttacatgcaaactgtgcaattggtttttttccatatttaatgcttcatatatgtattcaaatatttgatgtgacggaattttttaaagtttaaattcaaatatttgatgtgacggaattttttaaagtttgaagggaaataaacacggcctatggcgcaaaattgtactacaaactccctttcgcgagacgagagaatgcaacgccgtgccgctctggcggctggggcccaaccgcgaaaggcaatccatccctctctctccccgtcgcgtaaggccgctataaatcaacccgtcgcgggttgcattgcatcggtcggtctcggtcgctgccgtccctgtgcttggagtagacacgagccggtgacgcgcatatcgtcttctgctcgagatcgatcgatcgatcgcttggttggttgctgcgactgcgagggaggccgcgggtggtgaggaggattgtgcaaaaaaaatg

(19) United States
(12) Patent Application Publication
(10) Pub. No.: US 2015/0203864 A1
(43) Pub. Date: Jul. 23, 2015

(54) MYB55 PROMOTER AND USE THEREOF

(71) Applicant: University of Guelph, Guelph (CA)

(72) Inventors: Steven Rothstein, Guelph (CA); Yongmei Bi, Guelph (CA); Ashraf El-kereamy, Guelph (CA)

(73) Assignee: University of Guelph, Guelph, ON (CA)

(21) Appl. No.: 14/384,946

(22) PCT Filed: Mar. 13, 2013
(86) PCT No.: PCT/IB2013/051976

§ 371 Date: Sep. 12, 2014

Related U.S. Application Data
(60) Provisional application No. 61/610,294, filed on Mar. 13, 2012.

(51) Int. Cl. C12N 15/82

(52) U.S. Cl. CPC C12N 15/8271; C12N 15/8216

(57) ABSTRACT

The invention provides MYB55 promoter sequences that can advantageously be used to express a nucleotide sequence of interest in a plant, plant part or plant cell. Also provided are methods of increasing the expression of a nucleotide sequence of interest in a plant, plant part or plant cell in response to high temperature, abscisic acid, salicylic acid and/or methyl jasmonate. Further provided are methods of increasing the tolerance of a plant, plant part or plant cell to heat stress using the promoter sequences as described herein.

### RELATED APPLICATIONS

This application is a 35 U.S.C. §371 national stage application of International Application No. PCT/IB2013/051976, filed Mar. 13, 2013, which claims the benefit of priority from U.S. Provisional Application No. 61/610,294, filed Mar. 13, 2012, the contents of each of which are incorporated herein by reference in their entireties. The above-referenced International Application was published as International Publication No. WO 2013/136274 A1 on Sep. 19, 2013.

### STATEMENT REGARDING THE ELECTRONIC FILING OF SEQUENCES

A sequence listing in ASCII text format, submitted under 37 C.F.R. §1.821, entitled 9207-71TSv2_ST25.txt, 60,361 bytes in size, generated on Mar. 26, 2014, and filed electronically via EFS-Web, is provided in lieu of a paper copy.

### FIELD OF THE INVENTION

The present invention relates to methods of introducing and expressing nucleotide sequences in a plant, plant part or plant cell.

### BACKGROUND OF THE INVENTION

Plants are subject to various stress conditions that may adversely affect their productivity. For instance, heat stress may adversely affect various aspects of a plant's growth and development, including, but not limited to, fertility, seed germination, coleoptile growth, grain filing and/or fruit colour. See, e.g., Ashraf et al., ENVIRON. EXP. BOT. 34:275 (1994); Endo et al., PLANT CELL PHYSIOL. 50:1911 (2009); Jagadish et al., J. EXP. BOT. 61:143 (2010); Kolupaev et al., RUSSIAN J. PLANT PHYSIOL. 52:199 (2005); Lin et al., J. AGRIC. FOOD CHEM. 58:10545 (2010); Lin-Wang et al., PLANT CELL ENVIRON. 34(7):1176 (2011); Morita et al., ANN. BOT. 95:695 (2005)). Rice, which provides food to approximately half the world's population, may be particularly susceptible to heat stress. See Peng et al., PROC. NATL. ACAD. SCI. USA 101:9971 (2004) (describing decreased rice yields at the International Rice Institute in response to the global increase in nighttime temperatures between 1992 and 2003).

Although most heat response studies have focused on heat shock transcription factors and heat shock proteins, heat tolerance is a complex process that involves numerous genes, pathways and systems. Indeed, a variety of proteins, molecules and pathways have been shown to play a role in heat stress responses in cotton, wheat, corn and other plants.

MYB transcription factors regulate numerous processes during the plant life cycle and are classified into three major groups based upon the number of adjacent repeats in their binding domains: R1R2R3-MYB, R2R3-MYB, and R1-MYB. Most plant MYB transcription factors are of the R2R3 type, which are involved in a wide range of physiological responses such as regulation of the isopropanoid and flavonoid pathways, control of the cell cycle, root growth, and various defense and stress responses. Du et al., BIOCHEM. (MQSC) 74:1 (2009); Jin and Martin, PLANT MOL. BIOL. 41:577 (1999); Lee et al., MOL. PLANT MICROBE INTERACT. 14:527 (2001); Lin-Wang et al., BMC PLANT BIOL. 10:50 (2010); Mellway et al., PLANT PHYSIOL. 150:924 (2009); Mu et al., CELL RES. 19:1291 (2009); Raffaele et al., PLANT CELL 20:752 (2008); Stracke et al., CURR. OPIN. PLANT BIOL. 4:447 (2001); Sugimoto et al., PLANT CELL 12:2511 (2000); Yang and Klessig, PROC. NATL. ACAD. SCI. USA 93:14972 (1996)). Despite the large number of genes in the MYB family, the Arabidopsis MYB68 gene is the only member proposed to have a role in Arabidopsis heat tolerance. Feng et al., PLANT SCI. 167:1099 (2004) (describing a mutant MYB68 plant with reduced growth and higher lignin levels in the roots when grown at a high temperature).

### SUMMARY OF THE INVENTION

As one aspect, the invention provides an isolated nucleic acid comprising a nucleotide sequence that has promoter activity, the nucleotide sequence selected from the group consisting of: (a) a nucleotide sequence comprising a MYB55 promoter of the invention (e.g., SEQ ID NO: 1); (b) a nucleotide sequence comprising at least 500 consecutive nucleotides of the nucleotide sequence of SEQ ID NO: 1; (c) a nucleotide sequence that hybridizes to the complete complement of the nucleotide sequence of (a) or (b) under stringent conditions comprising a wash stringency of 50% Formamide with 5×Denhardt's solution, 0.5% SDS and 1×SSPE at 42° C.; and (d) a nucleotide sequence having at least 95% sequence identity to the nucleotide sequences of any of (a) to (c).

The invention further provides expression cassettes comprising an isolated nucleic acid of the invention operably associated with a nucleotide sequence of interest (e.g., a heterologous nucleotide sequence of interest).

As another aspect are vectors comprising an isolated nucleic acid or expression cassette of the invention.

As a further aspect are cells (e.g., plant cells) comprising an isolated nucleic acid, expression cassette, or vector of the invention.

Also provided are plant parts and transgenic plants comprising a plant cell of the invention.

As a further aspect are stably transformed transgenic plants comprising an isolated nucleic acid, expression cassette, or vector of the invention stably incorporated in its genome.

As another aspect are harvested products from the plants of the invention, and process products produced from such harvested products.

As still another aspect, the invention provides a crop comprising a plurality of the plants of the invention.

As yet a further aspect, the invention provides seed comprising an isolated nucleic acid, expression cassette, or vector of the invention stably incorporated in its genome.

As a further aspect, the invention provides a method of introducing a nucleic acid into a plant, plant part or plant cell, the method comprising transforming the plant, plant part or plant cell with an isolated nucleic acid comprising a MYB55 promoter, or an expression cassette or vector comprising the same.

As yet another aspect, the invention provides a method of introducing a nucleotide sequence of interest into a plant, the method comprising: (a) stably transforming a plant cell with an isolated nucleic acid comprising a MYB55 promoter, or an expression cassette or vector comprising the same; and (b) regenerating a stably transformed transgenic plant from the stably transformed plant cell of (a).

As still another aspect, the invention provides a method of expressing a nucleotide sequence of interest in a plant, plant part or plant cell, the method comprising transforming the plant, plant part or plant cell with an isolated nucleic acid, expression cassette or vector of the invention.

Yet as a further aspect, the invention provides a method of stably expressing a nucleotide sequence of interest in a plant, the method comprising: (a) stably transforming a plant cell with an isolated nucleic acid, expression cassette, or vector of the invention; regenerating a stably transformed transgenic plant from the stably transformed plant cell of (a); and (c) expressing the nucleotide sequence of interest in the plant.

In further aspects, the invention provides a method of increasing the expression of a nucleotide sequence of interest in response to heat stress or high temperature, the method comprising: transforming a plant, plant part or plant cell with an isolated nucleic acid, expression cassette or vector of the invention.

Another aspect of the invention is a method of increasing the expression of a nucleotide sequence of interest in response to heat stress or high temperature, the method comprising: (a) stably transforming a plant cell with an isolated nucleic acid, expression cassette, or vector of the invention; (b) regenerating a stably transformed plant from the stably transformed plant cell of (a).

Still further, the invention provides a method of increasing tolerance of a plant, plant part of plant cell to heat stress or high temperature, the method comprising: transforming a plant, plant part or plant cell with an isolated nucleic acid, expression cassette, or the vector of the invention, wherein the nucleotide sequence is operably associated with a nucleotide sequence of interest (e.g., a heterologous nucleotide sequence of interest) that provides increased tolerance to heat stress or high temperature.

As another aspect, the invention provides a method of increasing tolerance of a plant to heat stress or high temperature, the method comprising: (a) stably transforming a plant cell with an isolated nucleic acid, expression cassette, or vector of the invention, wherein the nucleotide sequence is operably associated with a nucleotide sequence of interest (e.g., heterologous nucleotide sequence of interest) that provides increased tolerance to heat stress or high temperature; and (b) regenerating a stably transformed plant from the stably transformed plant cell of (a).

Optionally, the methods of increasing tolerance to heat stress or high temperature, further comprise exposing the plant, plant part or plant cell to heat stress or high temperature.

In embodiments, the methods of the invention further comprise obtaining a progeny plant derived from a stably transformed transgenic plant, wherein the progeny plant comprises in its genome an isolated nucleic acid of the invention.

Also provided by the invention are stably transformed transgenic plants produced by the methods of the invention and seed produced therefrom, optionally wherein the seed comprises an isolated nucleic acid of the invention stably incorporated in its genome.

These and other aspects of the invention are described in more detail in the description of the invention set forth below.

### BRIEF DESCRIPTION OF THE DRAWINGS

FIG. 1A depicts an unrooted phylogenetic tree showing the similarity between Oryza sativa MYB55 (OsMYB55) and several of its homologues in other species.

FIGS. 1B-1F show various sequences described herein. FIG. 1B depicts the portion of the OsMYB55 promoter sequence (SEQ ID NO:1) that was used to drive expression of beta-glucuronidase (GUS) in the expression assays described in Example 3. FIG. 1C depicts the OsMYB55 promoter sequence (SEQ ID NO:2) and the adjoining 5′ untranslated region (UTR). FIG. 1D depicts the OsMYB55 gene sequence (SEQ ID NO:3), including the 5′ UTR, the promoter sequence, the coding region and the 3′ UTR. FIG. 1E depicts the nucleotide sequence of the OsMYB55 cDNA (SEQ ID NO:4). FIG. 1F depicts the amino acid sequence of the OsMYB55 protein (SEQ ID NO:5). Nucleotides residing in a promoter sequence are underlined. Nucleotides residing in a coding sequence are shown as uppercase letters. Amino acids residing in a DNA binding region are shown as bold, italicized letters.

FIG. 2A shows the OsMYB55 promoter sequence (SEQ ID NO:35), with cis-acting regulatory elements (CAREs) and transcription factor binding sites (TFBS) highlighted therein. “MeJa” refers to CAREs involved in MeJa responsiveness. “HSE” (SEQ ID NOS:36 and 37) refers to CAREs involved in heat stress responsiveness. “ABRE” refers to CAREs involved in abscisic acid responsiveness. “TCA” (SEQ ID NO:38) refers to CAREs involved in salicylic acid responsiveness. “LTR” refers to CAREs involved in low temperature responsiveness. “Skn-1” refers to CAREs involved in endosperm expression. “GCC Box” refers to binding sites for activating protein-2 (AP-2) transcription factors. “MBS box” refers to binding sites for MYB transcription factors. “W box” refers to binding sites for WRKY transcription factors. “DOF box” refers to binding sites for DNA-binding with one finger (DOF) transcription factors. The ATG start codon of the OsMYB55 coding sequence is indicated with asterisks.

FIG. 2B is a diagram that graphically depicts the location of potential CAREs in the OsMYB55 promoter region. “MeJa” refers to CAREs involved in MeJa responsiveness. “HSE” (SEQ ID NOS:36 and 37) refers to CAREs involved in heat stress responsiveness. “ABRE” refers to CAREs involved in abscisic acid responsiveness. The numbers below the diagram indicate the positions of the CAREs relative to the ATG initiation codon.

FIG. 3 depicts the relative gene expression levels of OsMSB55 at various stages in the life cycle of wild-type rice plants grown under normal growth conditions.

FIG. 4 is a graph showing the relative OsMYB55 transcript levels (mean±standard deviation, n=3) of leaves taken from wild-type rice plants grown under normal growth conditions for four weeks and then exposed to 45° C. for 0, 1, 6 or 24 hours.

FIG. 5 shows cross sections of the leaf sheaths (I, II), leaf blade (III) and roots (IV) taken from rice plants expressing GUS under the control of a 2134 base pair fragment (SEQ ID NO:1) of the OsMYB55 promoter region (OsMYB55promoter-GUS) that were grown under normal growth conditions for 4 weeks and then exposed to 29° C. (left) or to 45° C. (right) for 24 hours. Plant tissues were immersed in a solution containing 1 mg/ml 5-bromo-4-chloro-3-indolyl β-G-glucuronide (X-Gluc; Biosynth, Itasca, Ill.) to stain GUS protein, and cross sections were taken and visualized using a light microscope.

FIG. 6 is a graph showing OsMYB55 transcript levels in the leaves of wild-type rice plants (WT) and transgenic rice plants overexpressing OsMYB55 (OsMYB55-4; OsMYB55-11) grown under normal growth conditions for four weeks.

FIG. 7A shows seeds from wild-type rice plants (WT) and transgenic rice plants overexpressing OsMYB55 (OsMYB55-4; OsMYB55-11) following germination and four days of growth at 28° C. or 39° C.

FIG. 7B is a graph showing the coleoptile lengths (mean±standard deviation, n=3) of wild-type rice plants (WT) and transgenic rice plants overexpressing OsMYB55 following germination and four days of growth at 28° C. (Control) or 39° C. (High temperature). Each replicate consisted of 25 seedlings.

FIG. 8A shows wild-type rice plants (WT) and transgenic rice plants overexpressing OsMYB55 (55:4; 55:11) following germination under normal growth conditions and four weeks of growth in Turface® MVP® (PROFILE Products, LLC, Buffalo Grove, Ill.) under long daylight conditions with either normal temperature conditions (Control) or high temperature conditions (High temperature).

FIGS. 8B-8D are graphs showing the (B) plant heights, (C) above-ground vegetative biomasses and (D) root biomasses (mean±standard deviation, n=6) of wild-type rice plants (WT) and transgenic rice plants overexpressing OsMYB55 (OsMYB55-4; OsMYB55-11) following germination under normal growth conditions and four weeks of growth in Turface® MVP® (PROFILE Products, LLC, Buffalo Grove, Ill.) under long daylight conditions with either normal temperature conditions (Control) or high temperature conditions (High temperature).

FIG. 9A shows wild-type rice plants (WT) and transgenic rice plants overexpressing OsMYB55 (MYB55-4; MYB55-11) following four weeks of growth in peat-moss:vermiculite (1:4) under normal daylight conditions with either normal temperature conditions (“29”) or high temperature conditions (“35”).

FIG. 9B shows wild-type rice plants (WT) and transgenic rice plants overexpressing OsMYB55 (MYB55-4; MYB55-11) following four weeks of growth in peat-moss:vermiculite (1:4) under normal daylight conditions with high temperature conditions.

FIG. 9C is a graph showing the above-ground vegetative growth dry biomass (mean±standard deviation, n=6) of wild-type rice plants (WT) and transgenic rice plants overexpressing OsMYB55 (MYB55-4; MYB55-11) following four weeks of growth under normal daylight conditions with either normal temperature conditions (Control) or high temperature conditions (High temperature).

FIG. 9D is a graph showing plant height and leaf sheath length (mean±standard deviation, n=6) of wild-type rice plants (WT) and transgenic rice plants overexpressing OsMYB55 (MYB55-4; MYB55-11) following four weeks of growth under normal daylight conditions with high temperature conditions.

FIG. 10A shows the rice panicles of a wild-type rice plant (leftmost plant in each grouping) and transgenic rice plants overexpressing OsMYB55 (two rightmost plants in each grouping) following nine weeks of growth under normal daylight conditions with either normal temperature conditions (left) or high temperature conditions (right).

FIG. 10B shows the rice panicles of a wild-type rice plant following 11 weeks of growth under normal growth conditions.

FIG. 10C shows the rice panicles of a wild-type rice plant following 11 weeks of growth under long daylight conditions with high temperature conditions.

FIG. 10D shows the rice panicles of a wild-type rice plant following 11 weeks of growth under normal daylight conditions with high temperature conditions.

FIG. 10E shows the rice panicles of a wild-type rice plant following 17 weeks of growth under normal growth conditions.

FIG. 10F shows the rice panicles of a wild-type rice plant grown for 17 weeks under long daylight conditions with high temperature conditions.

FIG. 10G shows the rice panicles of a wild-type rice plant grown for 17 weeks under normal daylight conditions with high temperature conditions.

FIGS. 11A-11B are graphs showing the percent reduction in (A) total dry biomasses and (B) grain yields of wild-type rice plants (WT) and transgenic rice plants overexpressing OsMYB55 (OsMYB55-4; OsMYB55-11) grown under normal daylight conditions with high temperature conditions for four weeks and then grown under normal growth conditions until harvest (approximately 12 additional weeks) as compared to equivalent plants grown under normal growth conditions until harvest (approximately 16 weeks).

FIG. 12 is a graph showing the relative transcript levels (mean±standard deviation) of OsMYB55 in the leaves of wild-type rice plants (WT) and transgenic rice plants expressing OsMYB55 interference RNA (OsMYB55-RNAi) (OsMYB55::RNAi-12; OsMYB55::RNAi-16) grown under normal growth conditions. The OsMYB55 transcript level of OsMYB55::RNAi-12 was used as a reference value to calculate the relative transcripts levels.

FIG. 13A is a graph showing the total amino acid content (mean±standard deviation, n=3) of leaves from wild-type rice plants (WT) and transgenic rice plants overexpressing OsMYB55 (OsMYB55-4; OsMYB55-11) grown for four weeks under long daylight conditions with either normal temperature conditions (Control) or high temperature conditions (High temperature).

FIGS. 13B-13D are graphs showing the relative expression levels (mean±standard deviation, n=3) of (B) Oryza sativa glutamine synthetase (OsGs1;2), (C) Oryza sativa class I glutamine amidotransferase (OsGAT1) and (D) Oryza sativa glutamine decarboxylase 3 (OsGAD3) in the leaves of wild-type rice plants (WT) and transgenic rice plants overexpressing OsMYB55 (OsMYB55-4; OsMYB55-11) grown under long daylight conditions with normal temperature conditions for four weeks (Control) or grown under long daylight conditions with normal temperature conditions for four weeks and then exposed to 45° C. for 1, 6 or 24 hours. The results depicted in the graph are representative of similar results from three independent experiments.

FIG. 14A shows electrophoretic mobility shift assays using varying amounts of recombinant OsMYB55 (0-40 μg) and 200 ng of DNA containing one copy of a promoter region isolated from (I) OsGs1;2, (II) OsGAT1 or (Ill) OsGAD3.

FIG. 14B is a graph showing the expression levels (mean±standard deviation, n=6) of GUS in a transient gene expression assay wherein four-week-old tobacco plants were co-transformed with a vector comprising OsMYB55 and a GUS-reporting vector comprising GUS under the control of a promoter region isolated from OsGs1;2, OsGAT1 or OsGAD3.

FIG. 15A is a graph showing the glutamic acid content (mean±standard deviation) of leaves from wild-type rice plants (WT) and transgenic rice plants overexpressing OsMYB55 (OsMYB55-4; OsMYB55-11) following four weeks of growth under long daylight conditions with either normal temperature conditions (Control) or high temperature conditions (High temperature).

FIG. 15B is a graph showing the γ-aminobutyric acid (GABA) content (mean±standard deviation) of leaves from wild-type rice plants (WT) and transgenic rice plants overexpressing OsMYB55 (OsMYB55-4; OsMYB55-11) following four weeks of growth under long daylight conditions with either normal temperature conditions (Control) or high temperature conditions (High temperature).

FIG. 15C is a graph showing the arginine content (mean±standard deviation) of leaves from wild-type rice plants (WT) and transgenic rice plants overexpressing OsMYB55 (OsMYB55-4; OsMYB55-11) following four weeks of growth under long daylight conditions with either normal temperature conditions (Control) or high temperature conditions (High temperature).

FIG. 15D is a graph showing the proline content (mean±standard deviation) of leaves from wild-type rice plants (WT) and transgenic rice plants overexpressing OsMYB55 (OsMYB55-4; OsMYB55-11) following four weeks of growth under long daylight conditions with either normal temperature conditions (Control) or high temperature conditions (High temperature).

FIGS. 16A-16B are Venn diagrams representing the number of genes that were significantly (A) up-regulated or (B) down-regulated in wild-type rice plants (WT) and transgenic rice plants overexpressing OsMYB55 (OsMYB55) following four weeks of growth under normal growth conditions and then exposure to 45° C. for one hour.

FIGS. 17A-H shows the amino acid and coding sequences for various plant MYB55 homologues. FIG. 17A depicts the amino acid (SEQ ID NO:6) and cDNA (SEQ ID NO:14) sequences for a MYB55 homologue from Sorghum bicolor. FIG. 17B depicts the amino acid (SEQ ID NO:7) and cDNA (SEQ ID NO:15) sequences for a MYB55 homologue from Zea mays. FIG. 17C depicts the amino acid (SEQ ID NO:8) sequence for a MYB55 homologue from Vitis vinifera. FIG. 17D depicts the amino acid (SEQ ID NO:9) and cDNA (SEQ ID NO:16) sequences for a MYB55 homologue (previously designated MYB133) from Populus trichocarps. FIG. 17E depicts the amino acid (SEQ ID NO:10) and cDNA (SEQ ID NO:17) sequences for a MYB55 homologue (previously designated MYB24) from Malus×domestica. FIG. 17F depicts the amino acid (SEQ ID NO:11) and cDNA (SEQ ID NO:18) sequences for a MYB55 homologue (previously designated DcMYB4) from Glycine max. FIG. 17G depicts the amino acid (SEQ ID NO:12) and cDNA (SEQ ID NO:19) sequences for a MYB55 homologue from Daucus carota. FIG. 17H depicts the amino acid (SEQ ID NO:13) and cDNA (SEQ ID NO:20) sequences for a MYB55 homologue (previously designated MYB36) from Arabidopsis thaliana.

### DETAILED DESCRIPTION OF THE INVENTION

It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

### I. DEFINITIONS

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, ±5%, ±1%, ±0.5%, or even ±0.1% of the specified amount.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” when used in a claim or the description of this invention is not intended to be interpreted to be equivalent to “comprising.”

Unless indicated otherwise, the terms “heat stress” and “high temperature” (and similar terms) refer to exposing a plant, plant part or plant cell to elevated temperatures that are higher than is optimal for the plant species and/or variety and/or developmental stage. In representative embodiments, the plant, plant part or plant cell is exposed to a high temperature for an insufficient time to result in heat stress (e.g., reduced yield). In embodiments of the invention, the plant, plant part or plant cell is exposed to a high temperature for a sufficient time to result in heat stress.

For example, in embodiments of the invention, a plant can be exposed to high temperature for a sufficient period of time to produce heat stress in the plant and result in an adverse effect on plant function, development and/or performance, e.g., reduced cell division, size (e.g., reduced plant height) and/or number of plants and/or parts thereof and/or an impairment in an agronomic trait such as reduced yield, fruit drop, fruit size and/or number, seed size and/or number, quality of produce due to appearance and/or texture and/or increased flower abortion. Plants, plant parts and plant cells may be exposed or subjected to heat stress or high temperature under a variety of circumstances, e.g., a cultivated plant exposed to heat stress or high temperature due to ambient temperatures; a plant, plant part or plant cell exposed to heat stress or high temperature during harvesting, processing, storage and/or shipping; or a plant, plant part or plant cell exposed to heat stress or high temperature to achieve a desired effect (e.g., inducing the activity of a promoter of the invention to express an operably associated nucleotide sequence of interest).

Those skilled in the art will recognize that the terms “heat stress” and “high temperature” are not absolute and may vary with the plant species, plant variety, developmental stage, water availability, soil type, geographic location, day length, season, the presence of other abiotic and/or biotic stressors, and other parameters that are well within the level of skill in the art. Thus, while one species may be severely impacted by a temperature of 23° C., another species may not be impacted until at least 30° C., and the like. Typically, temperatures above 30° C. result in a significant reduction in the yields of most important crops.

In embodiments of the invention, exposure to heat stress or high temperature comprises exposing a plant, plant part or plant cell to temperatures of at least about 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C. or 55° C. In embodiments of the invention, exposure to heat stress or high temperature refers to temperatures from about 30° C. to about 45° C., 46° C., 47° C., 48° C., 49° C., 50° C., 51° C., 52° C., 53° C., 54° C. or 55° C.; from about 31° C. to about 45° C., 46° C., 47° C., 48° C., 49° C., 50° C., 51° C., 52° C., 53° C., 54° C. or 55° C.; from about 32° C. to about 45° C., 46° C., 47° C., 48° C., 49° C., 50° C., 51° C., 52° C., 53° C., 54° C. or 55° C.; from about 33° C. to about 45° C., 46° C., 47° C., 48° C., 49° C., 50° C., 51° C., 52° C., 53° C., 54° C. or 55° C.; from about 34° C. to about 45° C., 46° C., 47° C., 48° C., 49° C., 50° C., 51° C., 52° C., 53° C., 54° C. or 55° C.; from about 35° C. to about 45° C., 46° C., 47° C., 48° C., 49° C., 50° C., 51° C., 52° C., 53° C., 54° C. or 55° C.; from about 36° C. to about 45° C., 46° C., 47° C., 48° C., 49° C., 50° C., 51° C., 52° C., 53° C., 54° C. or 55° C.; from about 37° C. to about 45° C., 46° C., 47° C., 48° C., 49° C., 50° C., 51° C., 52° C., 53° C., 54° C. or 55° C.; from about 38° C. to about 45° C., 46° C., 47° C., 48° C., 49° C., 50° C., 51° C., 52° C., 53° C., 54° C. or 55° C.; from about 39° C. to about 45° C., 46° C., 47° C., 48° C., 49° C., 50° C., 51° C., 52° C., 53° C., 54° C. or 55° C.; or from about 40° C. to about 45° C., 46° C., 47° C., 48° C., 49° C., 50° C., 51° C., 52° C., 53° C., 54° C. or 55° C. In representative embodiments, the temperatures above refer to day-time temperatures.

In additional embodiments, exposure to heat stress or high temperature comprises exposing a plant, plant part or plant cell to night-time temperatures of about 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C. or 55° C. In embodiments of the invention, heat stress or high temperature refers to night-time temperatures from about 25° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 26° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 27° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 28° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 29° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 30° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 31° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 32° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 33° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 34° C. to about 40° C., 41° C., 42° C., 43° C., 44° C., 45° C., 46° C., 47° C., 48° C., 49° C., 50° C., 51° C., 52° C., 53° C., 54° C. or 55° C.; or from about 35° C. to about 40° C., 41° C., 42° C., 43° C., 44° C., 45° C., 46° C., 47° C., 48° C., 49° C., 50° C., 51° C., 52° C., 53° C., 54° C. or 55° C.

The plant, plant part or plant cell can be exposed to the heat stress or high temperature for any period of time. In exemplary embodiments, the plant, plant part or plant cell is exposed to heat stress or high temperature for a period of at least about 1, 2, 5, 10, 15, 20, 30, 40, 50, 60, 90 or 120 minutes or longer; at least about 1, 2, 5, 10, 15, 18, 24, 48, 72 or 96 hours or longer; at least about 1, 2, 3, 4, 7, 10, 14, 21 or 30 days or longer, at least about 1, 2, 3, 4, 5 or 6 weeks or longer; or at least about 1, 2, 3 or 4 months or longer.

The present invention encompasses heat stress or high temperature conditions produced by any combination of the temperatures and time periods described herein.

In representative embodiments, the plant, plant part or plant cell is exposed to heat stress or high temperature comprising a day-time temperature of about 35° C. and a night-time temperature of about 26° C., e.g., for a period of about one, two, three, four weeks, or longer. In embodiments of the invention, the plant, plant part or plant cell is subject to heat stress or high temperature comprising exposure to about 45° C. for a period of at least about 5, 10, 15, 20, 30, 40, 50, 60, 90 or 120 minutes or longer.

Those skilled in the art will appreciate that generally the plant, plant part or plant cell is exposed to a sub-lethal level of heat stress or high temperature (e.g., that is not lethal to the plant, plant part or plant cell).

The term “increased tolerance to heat stress,” “increasing tolerance to heat stress,” “increased tolerance to high temperature,” or “increasing tolerance to high temperature” (and similar terms) as used herein refers to the ability of a plant, plant part or plant cell exposed to heat stress or high temperature and comprising an isolated nucleic acid, expression cassette or vector comprising a promoter sequence as described herein to withstand a given heat stress or high temperature better than a control plant, plant part or plant cell (i.e., a plant, plant part or plant cell that does not comprise a nucleic acid, expression cassette or vector comprising a promoter sequence as described herein). Increased tolerance to heat stress or high temperature can be measured using a variety of parameters including, but not limited to, increased cell division, size (e.g., plant height) and/or number of plants and/or parts thereof and/or an improvement in an agronomic trait such as increased yield, fruit drop, fruit size and/or number, seed size and/or number and/or increased quality of produce due to appearance and/or texture and/or reduced flower abortion. In embodiments of the invention, increased tolerance to heat stress or high temperature can be assessed in terms of an increase in plant height, plant biomass (e.g., dry biomass) and/or grain yield. Increases in these indices (e.g., yield, plant size, plant height, plant biomass, grain yield, and the like) may indicate that there is an increase as compared with a control plant, plant part or plant cell that has not been subject to heat stress or high temperature and/or may indicate that there is an increase as compared with a control plant, plant part or plant cell that has been subject to heat stress or high temperature but does not comprise a nucleic acid, expression cassette or vector as described herein. In other words, there may be a reduction as compared with a plant, plant part or plant cell that has not been exposed to heat stress or high temperature but the decrease is less than in a plant, plant part or plant cell subject to the heat stress or high temperature that does not comprise a nucleic acid, expression cassette or vector as described herein.

“Yield” as used herein refers to the production of a commercially and/or agriculturally important plant, plant biomass (e.g., dry biomass), plant part (e.g., roots, tubers, seed, leaves, fruit, flowers), plant material (e.g., an extract) and/or other product produced by the plant (e.g., a recombinant polypeptide). In embodiments of the invention, “increased yield” is assessed in terms of an increase in plant height.

The term “modulate” (and grammatical variations) refers to an increase or decrease.

As used herein, the terms “increase,” “increases,” “increased,” “increasing” and similar terms indicate an elevation of at least about 25%, 50%, 75%, 100%, 150%, 200%, 300%, 400%, 500% or more.

As used herein, the terms “reduce,” “reduces,” “reduced,” “reduction” and similar terms mean a decrease of at least about 25%, 35%, 50%, 75%, 80%, 85%, 90%, 95%, 97% or more. In particular embodiments, the reduction results in no or essentially no (i.e., an insignificant amount, e.g., less than about 10% or even 5%) detectable activity or amount.

As used herein, the term “heterologous” means foreign, exogenous, non-native and/or non-naturally occurring.

As used here, “homologous” means native. For example, a homologous nucleotide sequence or amino acid sequence is a nucleotide sequence or amino acid sequence naturally associated with a host cell into which it is introduced, a homologous promoter sequence is the promoter sequence that is naturally associated with a coding sequence, and the like.

As used herein a “chimeric nucleic acid,” “chimeric nucleotide sequence” or “chimeric polynucleotide” comprises a promoter operably linked to a nucleotide sequence of interest that is heterologous to the promoter (or vice versa). In particular embodiments, the “chimeric nucleic acid,” “chimeric nucleotide sequence” or “chimeric polynucleotide” comprises a nucleic acid encoding a promoter sequence of the invention operably associated with a heterologous nucleotide sequence of interest.

A “promoter” is a nucleotide sequence that controls or regulates the transcription of a nucleotide sequence (i.e., a coding sequence) that is operatively associated with the promoter. The coding sequence may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

“Nucleotide sequence of interest” refers to any nucleotide sequence which, when introduced into a plant, confers upon the plant a desired characteristic, for example, increased tolerance to heat stress, high temperature and/or drought. The “nucleotide sequence of interest” can encode a polypeptide and/or an inhibitory polynucleotide (e.g., a functional RNA).

A “heterologous nucleotide sequence of interest” is heterologous (e.g., foreign) to the promoter with which it is operatively associated. For example, according to the present invention, the promoter sequences of the invention can be operatively associated with a heterologous nucleotide sequence of interest (e.g., a nucleotide sequence of interest that is not the native MYB55 coding sequence with which the MYB55 promoter is associated in its naturally occurring state).

A “functional” RNA includes any untranslated RNA that has a biological function in a cell, e.g., regulation of gene expression. Such functional RNAs include but are not limited to RNAi (e.g., siRNA, shRNA), miRNA, antisense RNA, ribozymes, RNA aptamers and the like.

By “operably linked” or “operably associated” as used herein, it is meant that the indicated elements are functionally related to each other, and are also generally physically related. For example, a promoter is operatively linked or operably associated to a coding sequence (e.g., nucleotide sequence of interest) if it controls the transcription of the sequence. Thus, the term “operatively linked” or “operably associated” as used herein, refers to nucleotide sequences on a single nucleic acid molecule that are functionally associated. Those skilled in the art will appreciate that the control sequences (e.g., promoter) need not be contiguous with the coding sequence, as long as they functions to direct the expression thereof. Thus, for example, intervening untranslated, yet transcribed, sequences can be present between a promoter and a coding sequence, and the promoter sequence can still be considered “operably linked” to the coding sequence.

By the term “express,” “expressing” or “expression” (or other grammatical variants) of a nucleic acid coding sequence, it is meant that the sequence is transcribed. In particular embodiments, the terms “express,” “expressing” or “expression” (or other grammatical variants) can refer to both transcription and translation to produce an encoded polypeptide.

“Wild-type” nucleotide sequence or amino acid sequence refers to a naturally occurring (“native”) or endogenous nucleotide sequence (including a cDNA corresponding thereto) or amino acid sequence.

The terms “nucleic acid,” “polynucleotide” and “nucleotide sequence” are used interchangeably herein unless the context indicates otherwise. These terms encompass both RNA and DNA, including cDNA, genomic DNA, partially or completely synthetic (e.g., chemically synthesized) RNA and DNA, and chimeras of RNA and DNA. The nucleic acid, polynucleotide or nucleotide sequence may be double-stranded or single-stranded, and further may be synthesized using nucleotide analogs or derivatives (e.g., inosine or phosphorothioate nucleotides). Such nucleotides can be used, for example, to prepare nucleic acids, polynucleotides and nucleotide sequences that have altered base-pairing abilities or increased resistance to nucleases. The present invention further provides a nucleic acid, polynucleotide or nucleotide sequence that is the complement (which can be either a full complement or a partial complement) of a nucleic acid, polynucleotide or nucleotide sequence of the invention. Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR §1.822 and established usage.

The nucleic acids and polynucleotides of the invention are optionally isolated. An “isolated” nucleic acid molecule or polynucleotide is a nucleic acid molecule or polynucleotide that, by the hand of man, exists apart from its native environment and is therefore not a product of nature. An isolated nucleic acid molecule or isolated polynucleotide may exist in a purified form or may exist in a non-native environment such as, for example, a recombinant host cell. Thus, for example, the term “isolated” means that it is separated from the chromosome and/or cell in which it naturally occurs. A nucleic acid or polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and polynucleotides of the invention can be considered to be “isolated.”

Further, an “isolated” nucleic acid or polynucleotide can be a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. The “isolated” nucleic acid or polynucleotide can exist in a cell (e.g., a plant cell), optionally stably incorporated into the genome. According to this embodiment, the “isolated” nucleic acid or polynucleotide can be foreign to the cell/organism into which it is introduced, or it can be native to an the cell/organism, but exist in a recombinant form (e.g., as a chimeric nucleic acid or polynucleotide) and/or can be an additional copy of an endogenous nucleic acid or polynucleotide. Thus, an “isolated nucleic acid molecule” or “isolated polynucleotide” can also include a nucleotide sequence derived from and inserted into the same natural, original cell type, but which is present in a non-natural state, e.g., present in a different copy number, in a different genetic context and/or under the control of different regulatory sequences than that found in the native state of the nucleic acid molecule or polynucleotide.

In representative embodiments, the “isolated” nucleic acid or polynucleotide is substantially free of cellular material (including naturally associated proteins such as histones, transcription factors, and the like), viral material, and/or culture medium (when produced by recombinant DNA techniques), or chemical precursors or other chemicals (when chemically synthesized). Optionally, in representative embodiments, the isolated nucleic acid or polynucleotide is at least about 10%, 20%, 30%, 40%, 50%, 60%, 70%, 80%, 90%, 95% or more pure.

As used herein, the term “recombinant” nucleic acid, polynucleotide or nucleotide sequence refers to a nucleic acid, polynucleotide or nucleotide sequence that has been constructed, altered, rearranged and/or modified by genetic engineering techniques. The term “recombinant” does not refer to alterations that result from naturally occurring events, such as spontaneous mutations, or from non-spontaneous mutagenesis.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in the cell, i.e., capable of nucleic acid replication under its own control. The term “vector” includes both viral and nonviral (e.g., plasmid) nucleic acid molecules for introducing a nucleic acid into a cell in vitro, ex vivo, and/or in vivo, and is optionally an expression vector. A large number of vectors known in the art may be used to manipulate, deliver and express polynucleotides. Vectors may be engineered to contain sequences encoding selectable markers that provide for the selection of cells that contain the vector and/or have integrated some or all of the nucleic acid of the vector into the cellular genome. Such markers allow identification and/or selection of host cells that incorporate and express the proteins encoded by the marker. A “recombinant” vector refers to a viral or non-viral vector that comprises one or more nucleotide sequences of interest (e.g., transgenes), e.g., two, three, four, five or more nucleotide sequences of interest.

Viral vectors have been used in a wide variety of gene delivery applications in cells, as well as living animal subjects. Plant viral vectors that can be used include, but are not limited to, Agrobacterium tumefaciens, Agrobacterium rhizcgenes and geminivirus vectors. Non-viral vectors include, but are not limited to, plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (e.g., delivery to specific tissues, duration of expression, etc.).

The term “fragment,” as applied to a nucleic acid or polynucleotide, will be understood to mean a nucleotide sequence of reduced length relative to the reference or full-length nucleotide sequence and comprising, consisting essentially of and/or consisting of contiguous nucleotides from the reference or full-length nucleotide sequence. Such a fragment according to the invention may be, where appropriate, included in a larger polynucleotide of which it is a constituent. In some embodiments, such fragments can comprise, consist essentially of, and/or consist of oligonucleotides having a length that greater than and/or is at least about 8, 10, 12, 15, 16, 17, 18, 19, 20, 25, 30, 35, 40, 45, 50, 75, 100, 125, 150, 175, 200, 250, 300, 350, 400, 450, 500, 600, 700, 800, 900, 1000, 1100, 1200, 1300, 1400, 1500, 1600, 1700, 1800, 1900, 2000, 2050, 2100, 2105, 2110, 2115, 2120, 2125, 2130, 2131, 2132, 2133, 2134, 2135, 2136, 2137, 2138 or 2139 nucleotides (optionally, contiguous nucleotides) or more from the reference or full-length nucleotide sequence, as long as the fragment is shorter than the reference or full-length nucleotide sequence. In representative embodiments, the fragment is a biologically active nucleotide sequence, as that term is described herein.

A “biologically active” nucleotide sequence is one that substantially retains at least one biological activity normally associated with the wild-type nucleotide sequence, for example, promoter activity, optionally inducible promoter activity in response to heat stress, high temperature, abscisic acid (ABA), methyl jasmonate (MeJa) and/or salicylic acid. In particular embodiments, the “biologically active” nucleotide sequence substantially retains all of the biological activities possessed by the unmodified sequence. By “substantially retains” biological activity, it is meant that the nucleotide sequence retains at least about 50%, 60%, 75%, 85%, 90%, 95%, 97%, 98%, 99%, or more, of the biological activity of the native nucleotide sequence (and can even have a higher level of activity than the native nucleotide sequence). Methods of measuring promoter activity are known in the art.

Two nucleotide sequences are said to be “substantially identical” to each other when they share at least about 60%, 70%, 75%, 80%, 85%, 90%, 95%, 97%, 98%, 99% or even 100% sequence identity. In embodiments of the invention, a “substantially identical” nucleotide sequence has about 1, 2, 3, 4, 5, 6, 7, 8, 9 or 10 nucleotide substitutions, insertions and/or deletions, taken individually or collectively, as compared with a reference sequence.

Two amino acid sequences are said to be “substantially identical” or “substantially similar” to each other when they share at least about 60%, 70%, 75%, 80%, 85%, 90%, 95%, 97%, 98%, 99% or even 100% sequence identity or similarity, respectively. In embodiments of the invention, a “substantially identical” amino acid sequence has about 1, 2, 3, 4, 5, 6, 7, 8, 9 or 10 amino acid substitutions, insertions and/or deletions, taken individually or collectively, as compared with a reference sequence. In embodiments of the invention, a “substantially similar” amino acid sequence has about 1, 2, 3, 4, 5, 6, 7, 8, 9 or 10 amino acid substitutions, insertions and/or deletions, taken individually or collectively, as compared with a reference sequence, where the amino acid substitutions can be conservative and/or non-conservative substitutions.

As used herein “sequence identity” refers to the extent to which two optimally aligned polynucleotide or polypeptide sequences are invariant throughout a window of alignment of components, e.g., nucleotides or amino acids.

As used herein “sequence similarity” is similar to sequence identity (as described herein), but permits the substitution of conserved amino acids (e.g., amino acids whose side chains have similar structural and/or biochemical properties), which are well-known in the art.

As is known in the art, a number of different programs can be used to identify whether a nucleic acid has sequence identity or an amino acid sequence has sequence identity or similarity to a known sequence. Sequence identity or similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Eva 35, 351-360 (1987); the method is similar to that described by Higgins & Sharp, CABIOS 5, 151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25, 3389-3402 (1997).

The CLUSTAL program can also be used to determine sequence similarity. This algorithm is described by Higgins et al. (1988) Gene 73:237; Higgins et al. (1989) CABIOS 5:151-153; Corpet et al. (1988) Nucleic Acids Res. 16: 10881-90; Huang et al. (1992) CABIOS 8: 155-65; and Pearson et al. (1994) Meth. Mol. Biol. 24: 307-331.

The alignment may include the introduction of gaps in the sequences to be aligned. In addition, for sequences which contain either more or fewer nucleotides than the nucleic acids disclosed herein, it is understood that in one embodiment, the percentage of sequence identity will be determined based on the number of identical nucleotides acids in relation to the total number of nucleotide bases. Thus, for example, sequence identity of sequences shorter than a sequence specifically disclosed herein, will be determined using the number of nucleotide bases in the shorter sequence, in one embodiment. In percent identity calculations relative weight is not assigned to various manifestations of sequence variation, such as, insertions, deletions, substitutions, etc.

Two nucleotide sequences can also be considered to be substantially identical when the two sequences hybridize to each other under stringent conditions. A nonlimiting example of “stringent” hybridization conditions include conditions represented by a wash stringency of 50% Formamide with 5×Denhardt's solution, 0.5% SDS and 1×SSPE at 42° C. “Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). In some representative embodiments, two nucleotide sequences considered to be substantially identical hybridize to each other under highly stringent conditions. Generally, highly stringent hybridization and wash conditions are selected to be about 5° C. lower than the thermal melting point (Tₘ) for the specific sequence at a defined ionic strength and pH.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

The polypeptides of the invention are optionally “isolated.” An “isolated” polypeptide is a polypeptide that, by the hand of man, exists apart from its native environment and is therefore not a product of nature. An isolated polypeptide may exist in a purified form or may exist in a non-native environment such as, for example, a recombinant host cell. The recombinant polypeptides of the invention can be considered to be “isolated.”

In representative embodiments, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. In particular embodiments, the “isolated” polypeptide is at least about 1%, 5%, 10%, 25%, 50%, 60%, 70%, 75%, 80%, 85%, 90%, 95%, 97%, 98%, 99% or more pure (w/w). In other embodiments, an “isolated” polypeptide indicates that at least about a 5-fold, 10-fold, 25-fold, 100-fold, 1000-fold, 10,000-fold, or more enrichment of the protein (w/w) is achieved as compared with the starting material. In representative embodiments, the isolated polypeptide is a recombinant polypeptide produced using recombinant nucleic acid techniques. In embodiments of the invention, the polypeptide is a fusion protein.

A “biologically active” polypeptide is one that substantially retains at least one biological activity normally associated with the wild-type polypeptide. In particular embodiments, the “biologically active” polypeptide substantially retains all of the biological activities possessed by the unmodified (e.g., native) sequence. By “substantially retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide).

“Introducing” in the context of a plant cell, plant tissue, plant part and/or plant means contacting a nucleic acid molecule with the plant cell, plant tissue, plant part, and/or plant in such a manner that the nucleic acid molecule gains access to the interior of the plant cell or a cell of the plant tissue, plant part or plant. Where more than one nucleic acid molecule is to be introduced, these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol.

The term “transformation” as used herein refers to the introduction of a heterologous and/or isolated nucleic acid into a cell. Transformation of a cell may be stable or transient. Thus, a transgenic plant cell, plant tissue, plant part and/or plant of the invention can be stably transformed or transiently transformed.

“Transient transformation” in the context of a polynucleotide means that a polynucleotide is introduced into the cell and does not integrate into the genome of the cell.

As used herein, “stably introducing,” “stably introduced,” “stable transformation” or “stably transformed” (and similar terms) in the context of a polynucleotide introduced into a cell, means that the introduced polynucleotide is stably integrated into the genome of the cell (e.g., into a chromosome or as a stable-extra-chromosomal element). As such, the integrated polynucleotide is capable of being inherited by progeny cells and plants.

“Genome” as used herein includes the nuclear and/or plastid genome, and therefore includes integration of a polynucleotide into, for example, the chloroplast genome. Stable transformation as used herein can also refer to a polynucleotide that is maintained extrachromosomally, for example, as a minichromosome.

As used herein, the terms “transformed” and “transgenic” refer to any plant, plant cell, plant tissue (including callus), or plant part that contains all or part of at least one recombinant or isolated nucleic acid, polynucleotide or nucleotide sequence. In representative embodiments, the recombinant or isolated nucleic acid, polynucleotide or nucleotide sequence is stably integrated into the genome of the plant (e.g., into a chromosome or as a stable extra-chromosomal element), so that it is passed on to subsequent generations of the cell or plant.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs plant cell tissue cultures, plant calli, plant clumps, and the like. As used herein, “shoot” refers to the above ground parts including the leaves and stems.

The term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ.

Any plant (or groupings of plants, for example, into a genus or higher order classification) can be employed in practicing the present invention including angiosperms or gymnosperms, monocots or dicots.

Exemplary plants include, but are not limited to corn (Zea mays), canola (Brassica napus, Brassica rapa ssp.), alfalfa (Medicago saliva), rice (Oryza sativa, including without limitation Indica and/or Japonica varieties), rape (Brassica napus), rye (Secale cereale), sorghum (Sorghum bicolor, Sorghum vulgare), sunflower (Helianthus annus), wheat (Triticum aestivum), soybean (Glycine max), tobacco (Nicotiana tobacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), apple (Malus pumila), blackberry (Rubus), strawberry (Fragaria), walnut (Juglans regia), grape (Vitis vinifera), apricot (Prunus armeniaca), cherry (Prunus), peach (Prunus persica), plum (Prunus domestica), pear (Pyrus communis), watermelon (Citrullus vulgaris). duckweed (Lemna), oats (Avena sativa), barley (Hordium vulgare), vegetables, ornamentals, conifers, and turfgrasses (e.g., for ornamental, recreational or forage purposes), and biomass grasses (e.g., switchgrass and miscanthus).

Vegetables include Solanaceous species (e.g., tomatoes; Lycopersicon esculentum), lettuce (e.g., Lactuea sativa), carrots (Caucus carota), cauliflower (Brassica oleracea), celery (apium graveolens), eggplant (Solanum melongena), asparagus (Asparagus officinalis), ochra (Abelmoschus esculentus), green beans (Phaseolus vulgaris), lima beans (Phaseolus limensis), peas (Lathyrus spp.), members of the genus Cucurbita such as Hubbard squash (C. Hubbard), Butternut squash (C. moschata), Zucchini (C. pepo), Crookneck squash (C. crookneck), C. argyrosperma, C. argyrosperma ssp sororia, C. digitata, C. ecuadorensis, C. foetidissima, C. lundelliana, and C. martinezii, and members of the genus Cucumis such as cucumber (Cucumis sativus), cantaloupe (C. cantalupensis), and musk melon (C. melo).

Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (dianthus caryophyllus), poinsettia (Euphorbia pulcherima), and chrysanthemum.

Conifers, which may be employed in practicing the present invention, include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Turfgrass include but are not limited to zoysiagrasses, bentgrasses, fescue grasses, bluegrasses, St. Augustinegrasses, bermudagrasses, bufallograsses, ryegrasses, and orchardgrasses.

Also included are plants that serve primarily as laboratory models, e.g., Arabidopsis.

### II. PROMOTER SEQUENCES

The invention provides nucleic acids comprising, consisting essentially of, or consisting of a MYB55 promoter of the invention. The term “MYB55 promoter” is intended to encompass the promoter sequences specifically disclosed herein (e.g., SEQ ID NO:1, nucleotides 1921 to 4061 of SEQ ID NO:2, nucleotides 2562 to 4061 of SEQ ID NO:2, or SEQ ID NO:2), and equivalents thereof (optionally, a biologically active equivalent) that have substantially identical nucleotide sequences to the MYB55 promoter sequences specifically disclosed herein, as well as fragments of a full-length MYB55 promoter (optionally, a biologically active fragment) and equivalents thereof (optionally, a biologically active equivalent) that have substantially identical nucleotide sequences to a fragment of the MYB55 promoter sequences specifically disclosed herein. The term “MYB55 promoter” includes sequences from rice as well as homologues from other plant species, including naturally occurring allelic variants, isoforms, splice variants, and the like, or can be partially or completely synthetic.

Homologues from other organisms, in particular other plants, can be identified using methods known in the art. For example, PCR and other amplification and hybridization techniques can be used to identify such homologues based on their sequence similarity to the sequences set forth herein.

Biological activities associated with the MYB55 promoter include, without limitation, the ability to control or regulate transcription of an operably associated coding sequence. Another non-limiting biological activity includes the ability to bind one or more transcription factors and/or RNA polymerase II. Other biological activities include without limitation the ability to be induced by heat stress, high temperature, ABA, MeJa and/or salicylic acid.

Thus, in exemplary embodiments, the isolated nucleic acid comprises, consists essentially of, or consists of SEQ ID NO:1, nucleotides 1921 to 4061 of SEQ IQ NO:2, nucleotides 2562 to 4061 of SEQ ID NO:2, or SEQ ID NO:2 or an equivalent of any of the foregoing (optionally, a biologically active equivalent).

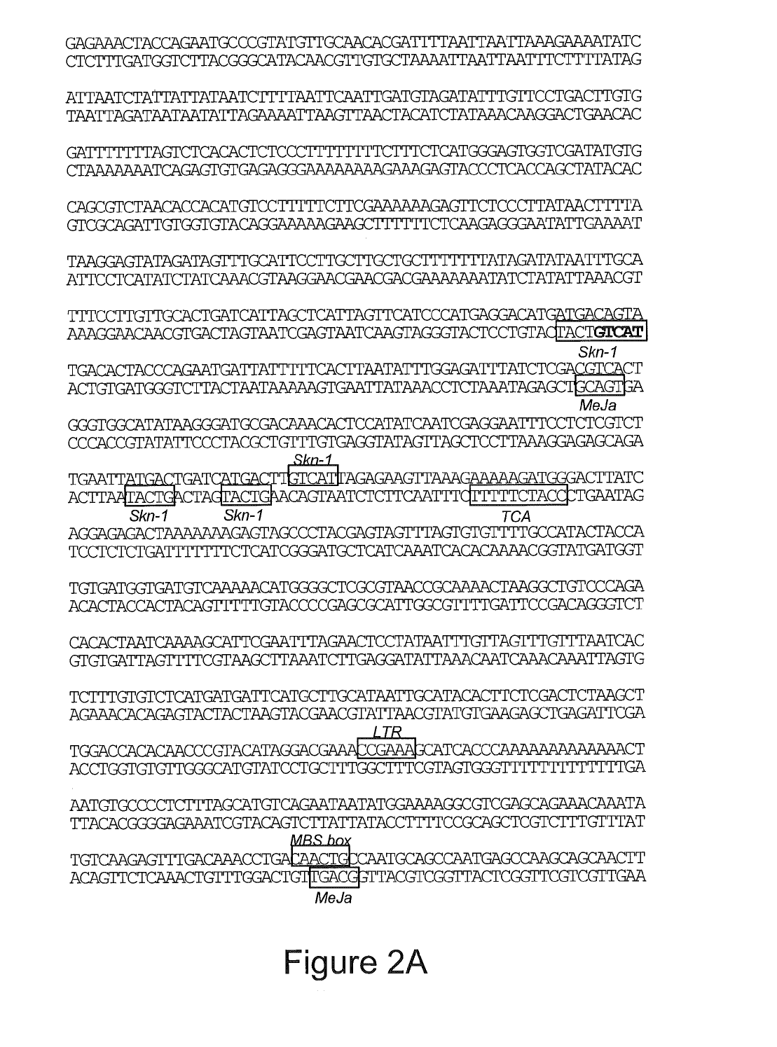

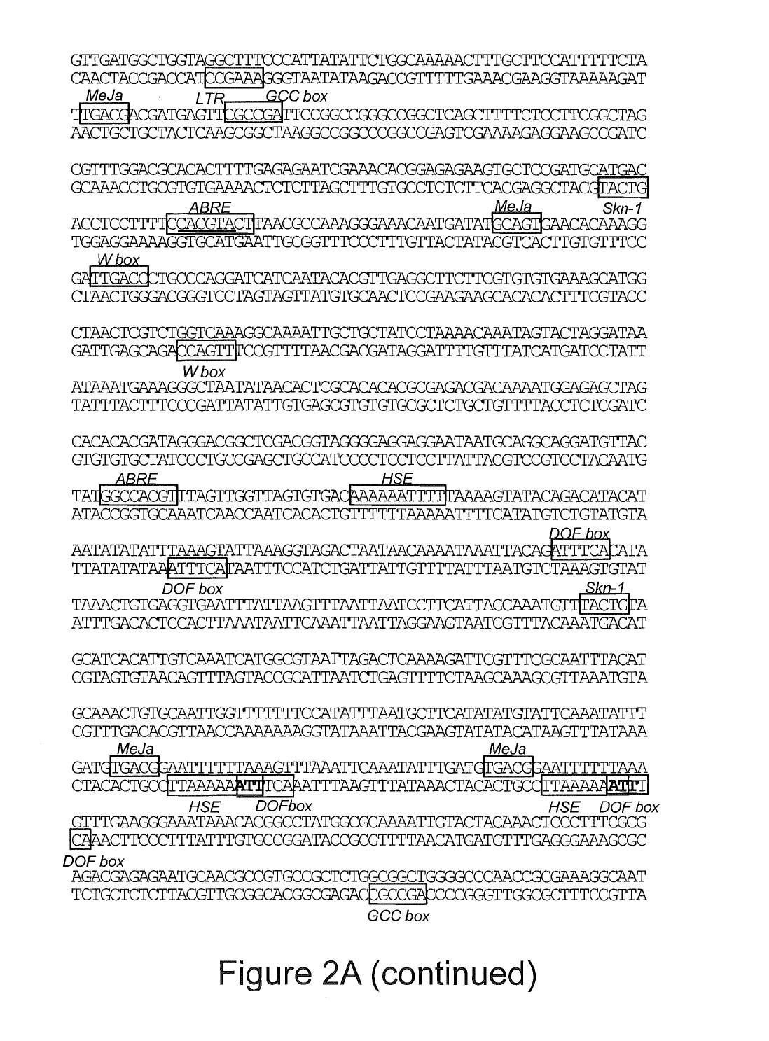

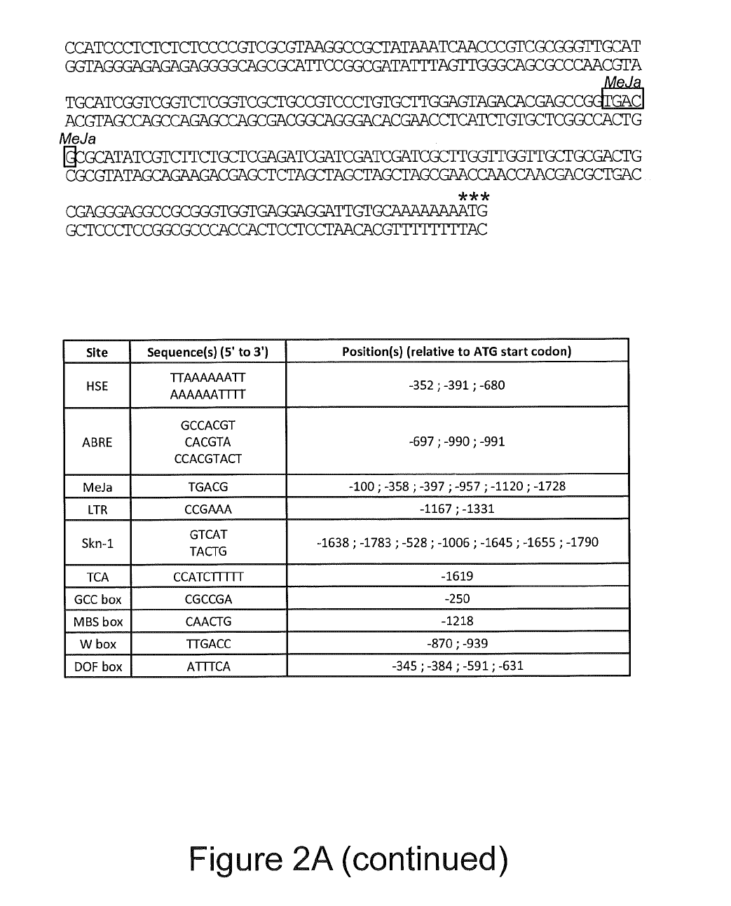

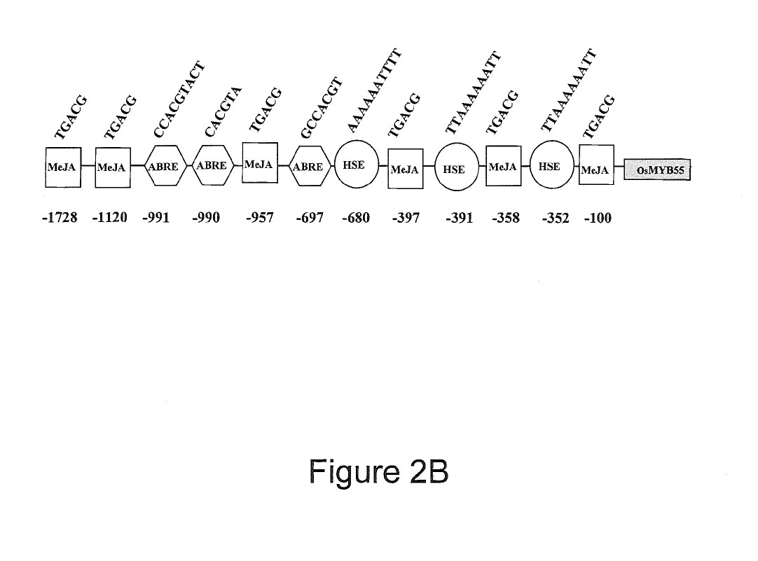

Equivalents of the MYB55 promoters of the invention encompass polynucleotides having substantial nucleotide sequence identity with the MYB55 promoter sequences specifically disclosed herein (e.g., SEQ ID NO:1, nucleotides 1921 to 4061 of SEQ ID NO:2, nucleotides 2562 to 4061 of SEQ ID NO:2, or SEQ ID NO:2) or fragments thereof, for example at least about 60%, 70%, 75%, 80%, 85%, 90%, 95%, 97%, 98% or 99% or more, and are optionally biologically active. In representative embodiments, there is no sequence variability in the TATA box, CAAT box, one or more of the Heat Shock Elements (HSE) (e.g., one, two or three), one or more of the ABA Responisve Elements (ABRE; e.g., one two or three), one or more of the Methyl Jasonate (MeJa) response elements (e.g., one, two, three, four or five), the Low Temperature Responsiveness (LTR) element, one or more of the DOF binding sites (“DOF box”; e.g., one, two or three), the MYB binding site (“MBS Box”), the AP-2 binding site (“GCC Box”, one or more of the WRKY binding sites (“W box”; e.g., one or two), one or more of the Skn-1 binding sites (e.g., one, two, three, four or five) and/or the TCA-element (see, e.g., the schematic in FIGS. 2A and 2B), i.e., these sequences are conserved and any sequence variability falls outside these regions.

The MYB55 promoters of the invention also include polynucleotides that hybridize to the complete complement of the MYB55 promoter sequences specifically disclosed herein (e.g., SEQ ID NO:1, nucleotides 1921 to 4061 of SEQ ID NO:2, nucleotides 2562 to 4061 of SEQ ID NO:2, or SEQ ID NO:2) or fragments thereof under stringent hybridization conditions as known by those skilled in the art and are optionally biologically active.

The MYB55 promoter sequences encompass fragments (optionally, biologically active fragments) of the MYB55 promoter sequences specifically disclosed herein (e.g., SEQ ID NO:1, nucleotides 1921 to 4061 of SEQ ID NO:2, nucleotides 2562 to 4061 of SEQ ID NO:2, or SEQ ID NO:2) and equivalents thereof. The length of the MYB55 promoter fragments is not critical. Illustrative fragments comprise at least and/or are greater than about 8, 10, 12, 15, 16, 17, 18, 19, 20, 25, 30, 35, 40, 45, 50, 75, 100, 125, 150, 175, 200, 250, 300, 350, 400, 450, 500, 600, 700, 800, 900, 1000, 1100, 1200, 1300, 1400, 1500, 1600, 1700, 1800, 1900, 2000, 2050, 2100, 2105, 2110, 2115, 2120, 2125, 2130, 2131, 2132, 2133, 2134, 2135, 2136, 2137, 2138 or 2139 or more nucleotides (optionally, contiguous nucleotides) of the full-length sequence.

In representative embodiments, the MYB55 promoter sequence comprises the TATA box sequence, the CAAT box sequence, one or more of the HSE elements (e.g., one, two or three), one or more of the ABRE elements (e.g., one two or three), one or more of the MeJa elements (e.g., one, two, three, four or five), the LTR element, one or more of the DOF binding sites (“DOF box”; e.g., one, two or three), the MYB binding site (“MBS Box”), the AP-2 binding site (“GCC Box”, one or more of the WRKY binding sites (“W box”; e.g., one or two), one or more of the Skn-1 binding sites (e.g., one, two, three, four or five) and/or the TCA-element (see, e.g., the schematic in FIGS. 2A and 2B), i.e., these sequences are conserved and any sequence variability falls outside these regions.

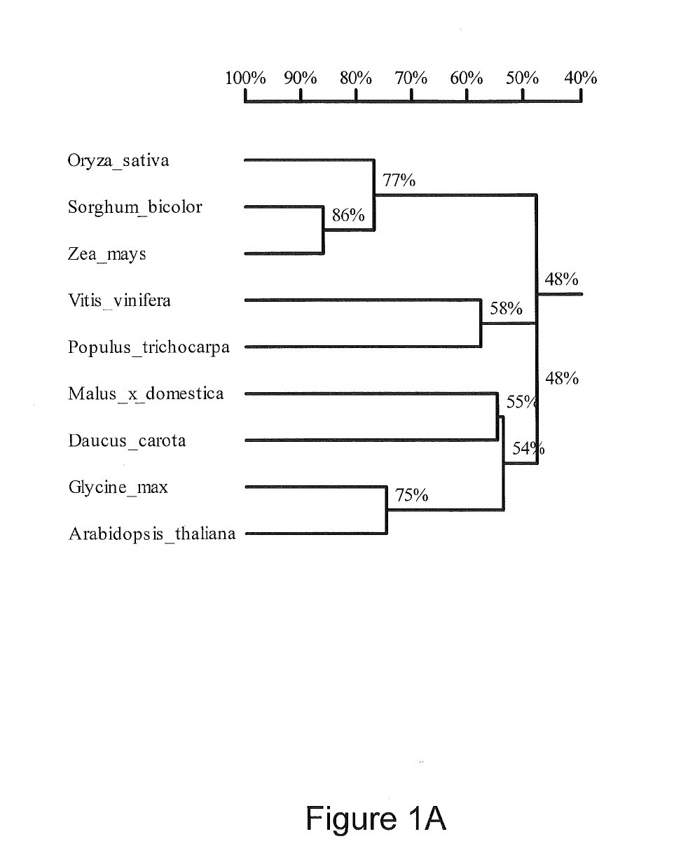

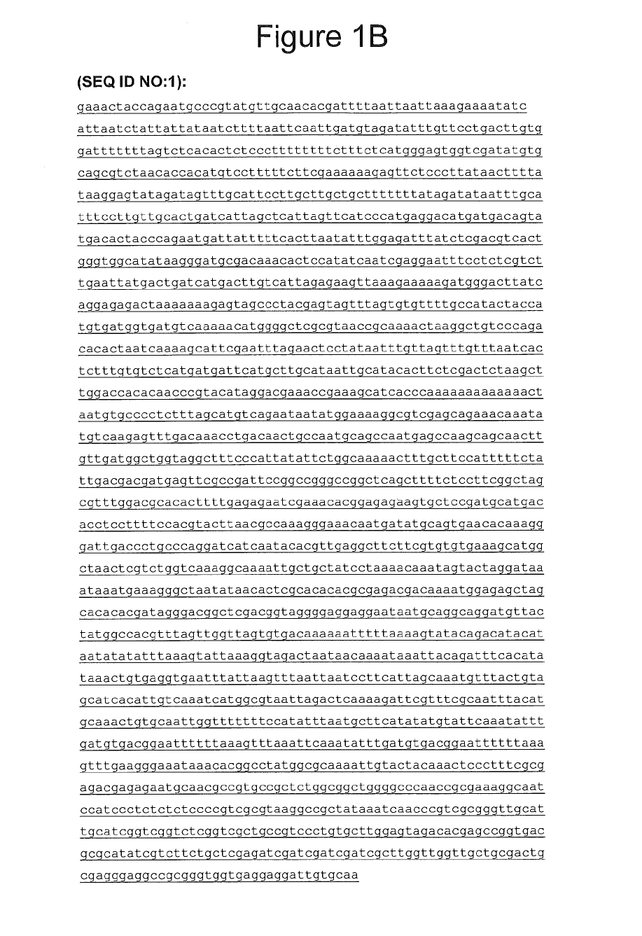

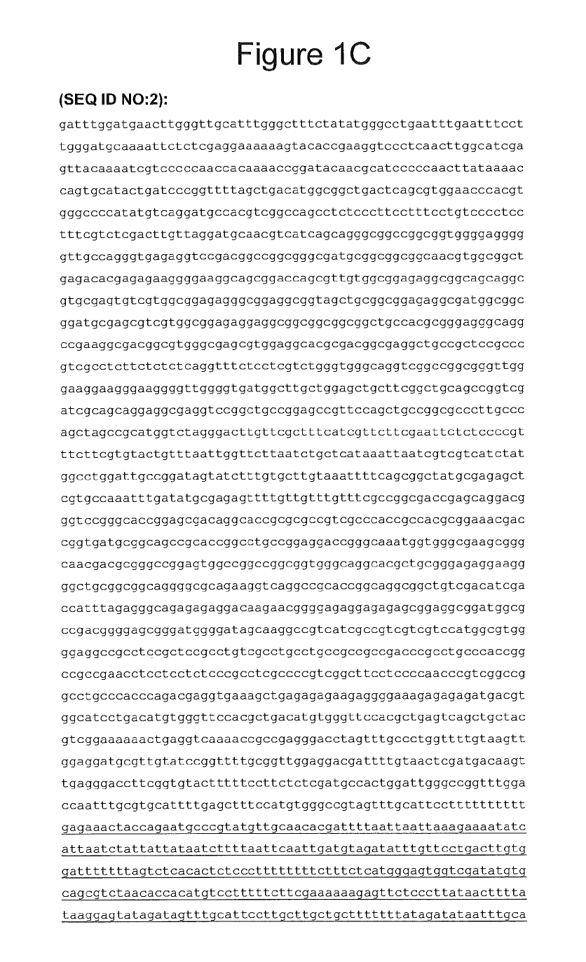

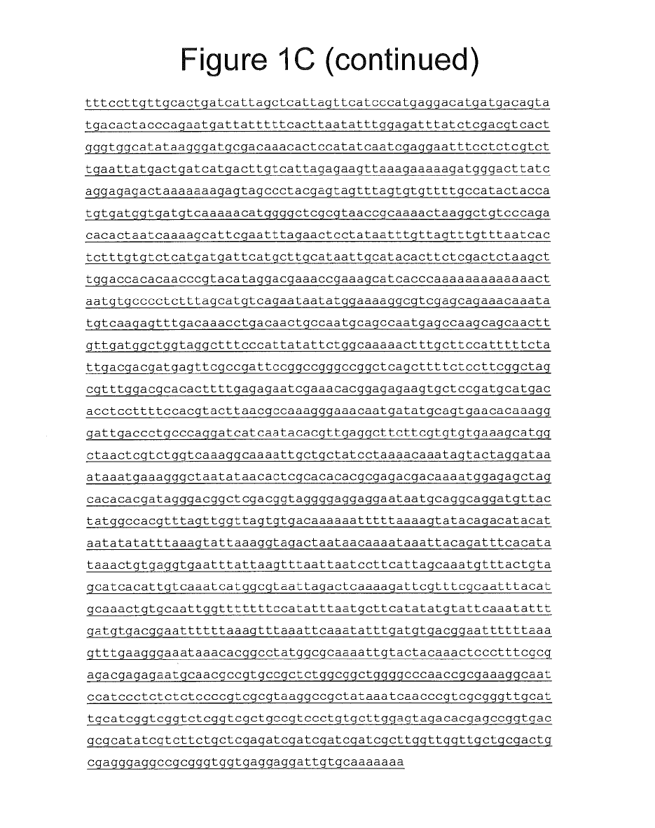

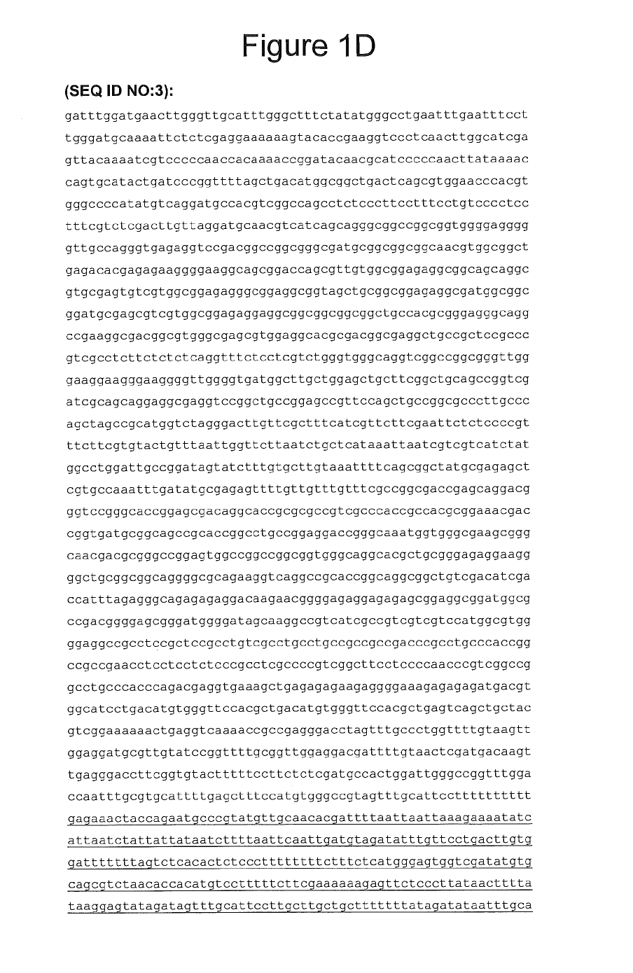

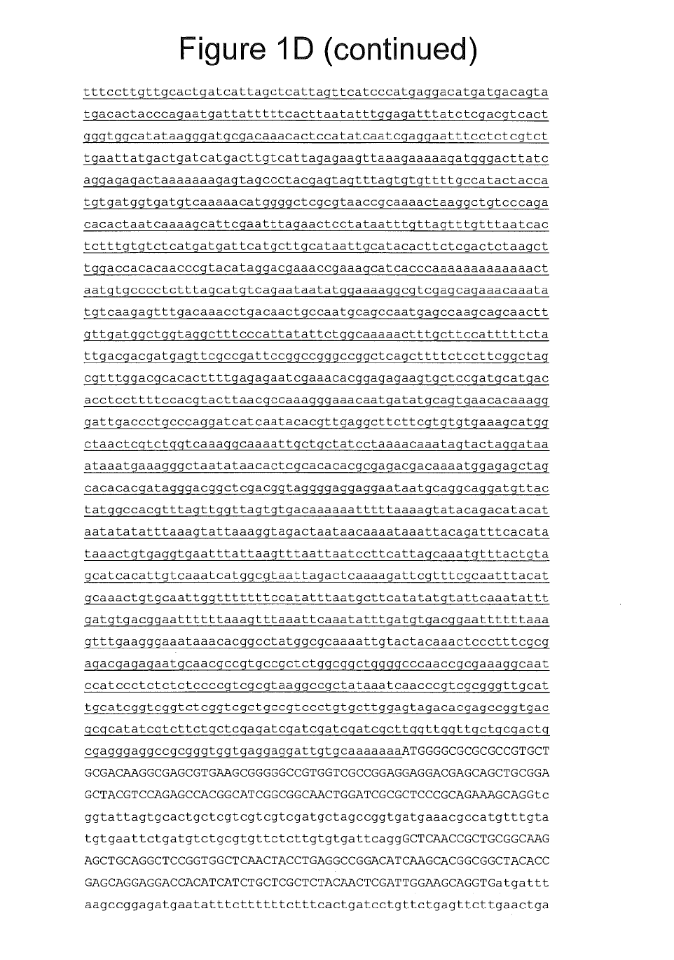

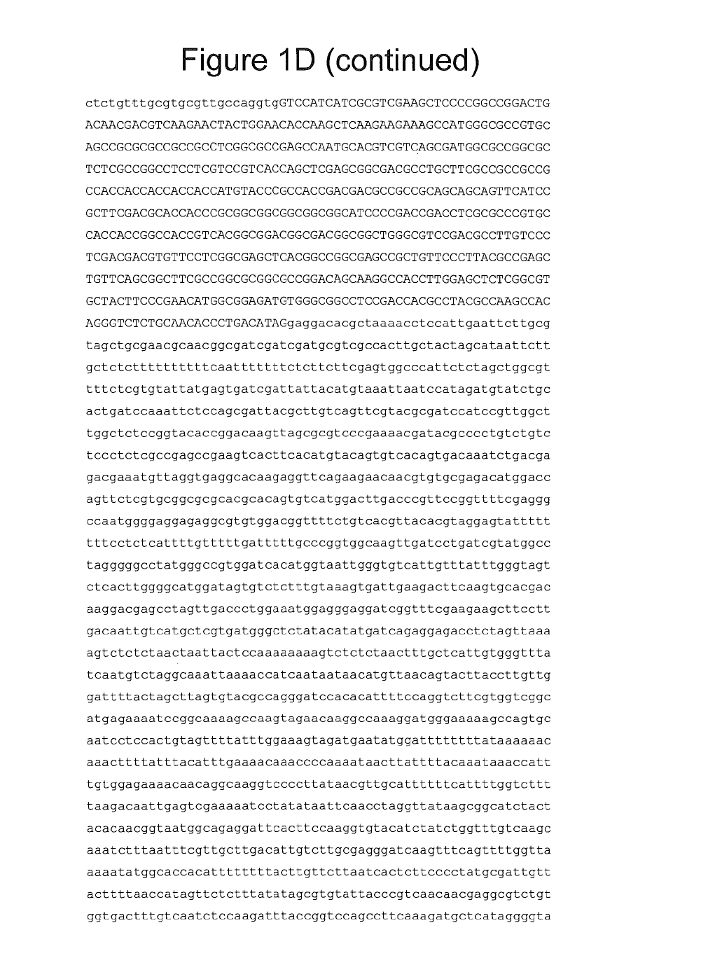

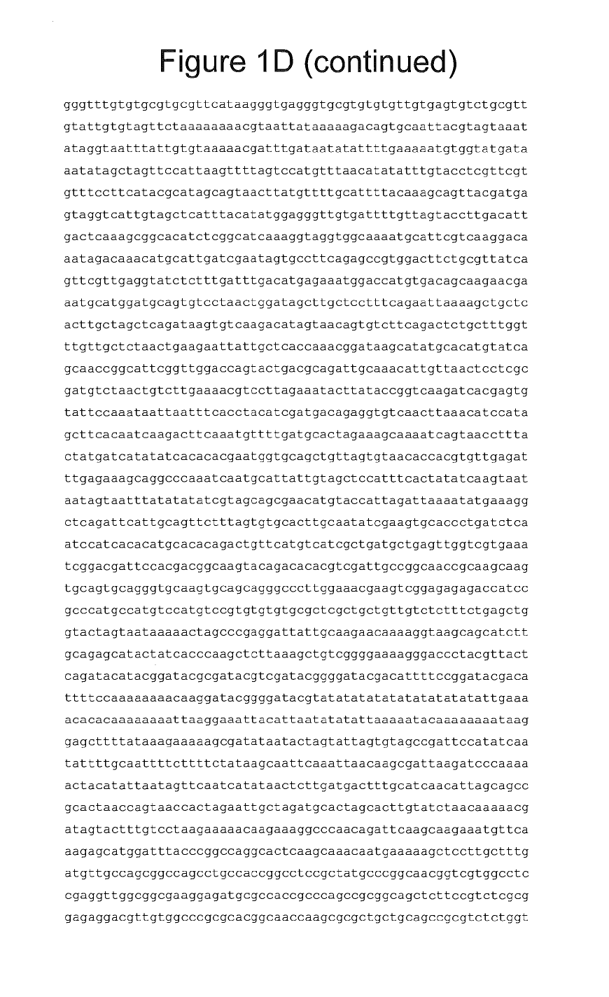

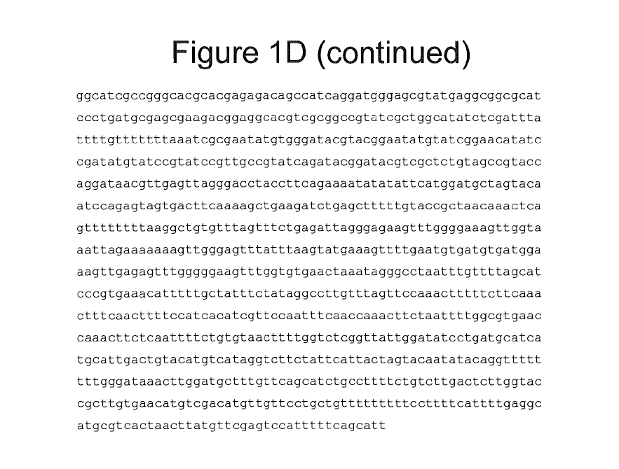

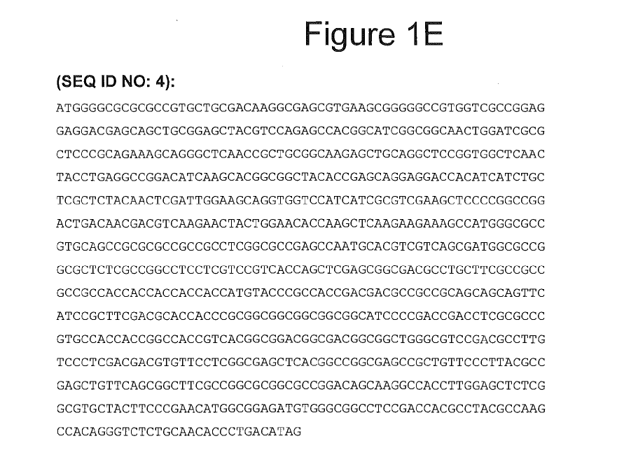

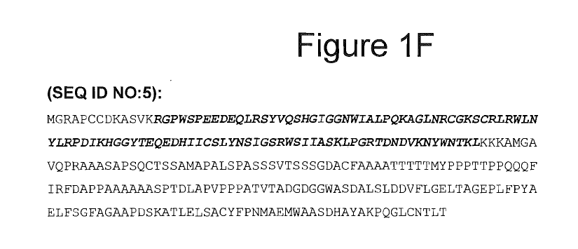

In embodiments of the invention, the nucleic acid comprising the MYB55 promoter does not include any of the MYB55 coding region (e.g., nucleotides 4062 to 5126 of SEQ ID NO:3; FIG. 1D). In embodiments of the invention, the nucleotide sequence of interest does not encode a MYB55 polypeptide (e.g., SEQ ID NO: 5; FIG. 1F). In embodiments of the invention, the nucleotide sequence of interest encodes a MYB55 polypeptide.

Accordingly, in representative embodiments, the invention provides a nucleic acid (e.g., a recombinant or isolated nucleic acid) comprising, consisting essentially of, or consisting of a nucleotide sequence selected from the group consisting of: (a) SEQ ID NO:1, nucleotides 1921 to 4061 of SEQ ID NO:2, nucleotides 2562 to 4061 of SEQ ID NO:2, or SEQ ID NO:2; (b) a nucleotide sequence comprising at least about 8, 10, 12, 15, 16, 17, 18, 19, 20, 25, 30, 35, 40, 45, 50, 75, 100, 125, 150, 175, 200, 250, 300, 350, 400, 450, 500, 600, 700, 800, 900, 1000, 1100, 1200, 1300, 1400, 1500, 1600, 1700, 1800, 1900, 2000, 2050, 2100, 2105, 2110, 2115, 2120, 2125, 2130, 2131, 2132, 2133, 2134, 2135, 2136, 2137, 2138 or 2139 or more nucleotides (optionally, contiguous nucleotides) of SEQ ID NO:1, nucleotides 1921 to 4061 of SEQ ID NO:2, nucleotides 2562 to 4061 of SEQ ID NO2, or SEQ ID NO:2; (c) a nucleotide sequence that hybridizes to the complete complement of the nucleotide sequence of (a) or (b) under stringent hybridization conditions; and (d) a nucleotide sequence having at least about 60%, 70%, 75%, 80%, 85%, 90%, 95%, 97%, 98%, 99% sequence identity to the nucleotide sequences of any of (a) to (c). In representative embodiments, the nucleotide sequence is a biologically active promoter sequence (e.g., has promoter activity) and is optionally induced by heat stress, high temperature, ABA, salicylic acid and/or MeJa.

In embodiments of the invention, the nucleotide sequence comprises, consists essentially of, or consists of the nucleotide sequence of SEQ ID NO:1, nucleotides 1921 to 4061 of SEQ ID NO:2, nucleotides 2562 to 4061 of SEQ ID NO:2, or SEQ ID NO:2.

In embodiments, the MYB55 promoter of the invention is operably associated with a nucleotide sequence of interest, which is optionally a heterologous nucleotide sequence of interest. According to this embodiment, the MYB55 promoter controls or regulates expression (e.g., transcription and, optionally, translation) of the nucleotide sequence of interest.

The invention also provides an expression cassette comprising a MYB55 promoter sequence of the invention, optionally in operable association with a nucleotide sequence of interest. The expression cassette can further have a plurality of restriction sites for insertion of a nucleotide sequence of interest to be operably linked to the regulatory regions. In particular embodiments, the expression cassette comprises more than one (e.g., two, three, four or more) nucleotide sequences of interest.

The expression cassettes of the invention may further comprise a transcriptional termination sequence. Any suitable termination sequence known in the art may be used in accordance with the present invention. The termination region may be native with the transcriptional initiation region, may be native with the nucleotide sequence of interest, or may be derived from another source. Convenient termination regions are available from the Ti-plasmid of A. tumefaciens, such as the octopine synthetase and nopaline synthetase termination regions. See also, Guerineau et al., Mol. Gen. Genet. 262, 141 (1991); Proudfoot, Cell 64, 671 (1991); Sanfacon et al., Genes Dev. 5,141 (1991); Mogen et al., Plant Cell 2, 1261 (1990); Munroe et al., Gene 91, 151 (1990); Sallas et al., Nucleic Acids Res. 17, 7891 (1989); and Joshi et al., Nucleic Acids Res. 15, 9627 (1987). Additional exemplary termination sequences are the pea RubP carboxylase small subunit termination sequence and the Cauliflower Mosaic Virus 35S termination sequence. Other suitable termination sequences will be apparent to those skilled in the art.

Further, in particular embodiments, the nucleotide sequence of interest is operably associated with a translational start site. The translational start site can be derived from the MYB55 coding sequence or, alternatively, can be the native translational start site associated with a heterologous nucleotide sequence of interest, or any other suitable translational start codon.

In illustrative embodiments, the expression cassette includes in the 5′ to 3′ direction of transcription, a promoter, a nucleotide sequence of interest, and a transcriptional and translational termination region functional in plants.

Those skilled in the art will understand that the expression cassettes of the invention can further comprise enhancer elements and/or tissue preferred elements in combination with the promoter.

Further, in some embodiments, it is advantageous for the expression cassette to comprise a selectable marker gene for the selection of transformed cells. Suitable selectable marker genes include without limitation genes encoding antibiotic resistance, such as those encoding neomycin phosphotransferase II (NEO) and hygromycin phosphotransferase (HPT), as well as genes conferring resistance to herbicidal compounds. Herbicide resistance genes generally code for a modified target protein insensitive to the herbicide or for an enzyme that degrades or detoxifies the herbicide in the plant before it can act. See, DeBlock et al., EMBO J. 6, 2513 (1987); DeBlock et al., Plant Physiol. 91, 691 (1989); Fromm et al., BioTechnology 8, 833 (1990); Gordon-Kamm et al., Plant Cell 2, 603 (1990). For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

Selectable marker genes that can be used according to the present invention further include, but are not limited to, genes encoding: neomycin phosphotransferase II (Fraley et al., CRC Critical Reviews in Plant Science 4, 1 (1986)); cyanamide hydratase (Maier-Greiner et al., Proc. Natl. Acad. Sci. USA 88, 4250 (1991)); aspartate kinase; dihydrodipicolinate synthase (Pert et al., BioTechnology 11, 715 (1993)); the bar gene (Toki et al., Plant Physiol. 100, 1503 (1992); Meagher et al., Crop Sci. 36, 1367 (1996)); tryptophane decarboxylase (Goddijn et at, Plant Mol. Biol. 22, 907 (1993)); neomycin phosphotransferase (NEO; Southern et al., J. Mol. Appl. Gen. 1, 327 (1982)); hygromycin phosphotransferase (HPT or HYG; Shimizu et al., Mol. Cell. Biol. 6, 1074 (1986)); dihydrofolate reductase (DHFR; Kwok et al., Proc. Natl. Acad. Sot USA 83, 4552 (1986)); phosphinothricin acetyltransferase (DeBlock et al., EMBQ J. 6, 2513 (1987)); 2,2-dichloropropionic acid dehalogenase (Buchanan-Wollatron et al., J. Cell. Biochem. 13D, 330 (1989)); acetohydroxyacid synthase (U.S. Pat. No. 4,761,373 to Anderson et al.; Haughn et al., Mol. Gen. Genet. 221, 266 (1988)); 5-enolpyruvyl-shikimate-phosphate synthase (aroA; Comai et al., Nature 317, 741 (1985)); haloarylnitrilase (WO 87/04181 to Stalker et al.); acetyl-coenzyme A carboxylase (Parker et al, Plant Physiol. 92, 1220 (1990)); dihydropteroate synthase (sull; Guerineau et al., Plant Mol. Biol. 15, 127 (1990)); and 32 kDa photosystem II polypeptide (psbA; Hirschberg et al., Science 222, 1346 (1983)).

Also included are genes encoding resistance to: chloramphenicol (Herrera-Estrella et al., EMBO J. 2, 987 (1983)); methotrexate (Herrera-Estrella et al., Nature 303, 209 (1983); Meijer et al., Plant Mol. Biol. 16, 807 (1991)); hygromycin (Waldron et al., Plant Mol. Biol. 5, 103 (1985); Zhijian et al., Plant Science 108, 219 (1995); Meijer et al., Plant Mol. Bio. 16, 807 (1991)); streptomycin (Jones et al., Mol. Gen. Genet. 210, 86 (1987)); and spectinomycin (Bretagne-Sagnard et al., Transgenic Res. 5, 131 (1996)); bleomycin (Hille et al., Plant Mol. Biol. 7, 171 (1986)); sulfonamide (Guerineau et al., Plant Mol. Bio. 15, 127 (1990); bromoxynil (Stalker et al., Science 242, 419 (1988)); 2,4-D (Streber et al., Bio/Technology 7, 811 (1989)); phosphinothricin (DeBlock et al., EMBO J. 6, 2513 (1987)); spectinomycin (Bretagne-Sagnard and Chupeau, Transgenic Research 5, 131 (1996)).

Other selectable marker genes include the pat gene (for bialaphos and phosphinothricin resistance), the ALS gene for imidazolinone resistance, the HPH or HYG gene for hygromycin resistance, the Hm1 gene for resistance to the Hc-toxin, and other selective agents used routinely and known to one of ordinary skill in the art. See generally, Yarranton, Curr. Opin. Biotech. 3, 506 (1992); Chistopherson et al., Proc. Natl. Acad. Sci. USA 89, 6314 (1992); Yao et al., Cell 71, 63 (1992); Reznikoff, Mol. Microbiol. 6, 2419 (1992); BARKLEY ET AL., THE OPERON 177-220 (1980); Hu et al., Cell 48, 555 (1987); Brown et al., Cell 49, 603 (1987); Figge et al., Cell 52, 713 (1988); Deuschle et al., Proc. Natl. Acad. Sci. USA 86, 5400 (1989); Fuerst at al., Proc. Natl. Acad. Sci. USA 86, 2549 (1989); Deuschle et al., Science 248, 480 (1990); Labow et al., Mol. Cell. Biol. 10, 3343 (1990); Zambretti et al., Proc. Natl. Acad. Sci. USA 89, 3952 (1992); Bairn et al., Proc. Natl. Acad. Sci. USA 88, 5072 (1991); Wyborski et al., Nuc. Acids Res. 19, 4647 (1991); Hillenand-Wissman, Topics in Mol. And Struc. Biol. 10, 143 (1989); Degenkolb et al., Antimicrob. Agents Chemother. 35, 1591 (1991); Kleinschnidt et al., Biochemistry 27, 1094 (1988); Gatz et al., Plant J. 2, 397 (1992); Gossen et al., Proc. Natl. Acad. Sci. USA 89, 5547 (1992); Oliva at al., Antimicrob. Agents Chemother. 36, 913 (1992); HLAVKA ET AL., HANDBOOK OF EXPERIMENTAL PHARMACOLOGY 78 (1985); and Gill et al., Nature 334, 721 (1988).

The nucleotide sequence of interest can additionally be operably linked to a sequence that encodes a transit peptide that directs expression of an encoded polypeptide of interest to a particular cellular compartment. Transit peptides that target protein accumulation in higher plant cells to the chloroplast, mitochondrion, vacuole, nucleus, and the endoplasmic reticulum (for secretion outside of the cell) are known in the art. Transit peptides that target proteins to the endoplasmic reticulum are desirable for correct processing of secreted proteins. Targeting protein expression to the chloroplast (for example, using the transit peptide from the RubP carboxylase small subunit gene) has been shown to result in the accumulation of very high concentrations of recombinant protein in this organelle. The pea RubP carboxylase small subunit transit peptide sequence has been used to express and target mammalian genes in plants (U.S. Pat. Nos. 5,717,084 and 5,728,925 to Herrera-Estrella et al.). Alternatively, mammalian transit peptides can be used to target recombinant protein expression, for example, to the mitochondrion and endoplasmic reticulum. It has been demonstrated that plant cells recognize mammalian transit peptides that target endoplasmic reticulum (U.S. Pat. Nos. 5,202,422 and 5,639,947 to Hiatt et al.).

Further, the expression cassette can comprise a 5′ leader sequence that acts to enhance expression (transcription, post-transcriptional processing and/or translation) of an operably associated nucleotide sequence of interest. Leader sequences are known in the art and include sequences from: picornavirus leaders, e.g., EMCV leader (Encephalomyocarditis 5′ noncoding region; Elroy-Stein et al., Proc. Natl. Acad. Sci USA, 86, 6126 (1989)).; potyvirus leaders, e.g., TEV leader (Tobacco Etch Virus; Allison et al., Virology, 154, 9 (1986)); human immunoglobulin heavy-chain binding protein (BiP; Macajak and Sarnow, Nature 353, 90 (1991)); untranslated leader from the coat protein mRNA of alfalfa mosaic virus (AMV RNA 4; Jobling and Gehrke, Nature 325, 622 (1987)); tobacco mosaic virus leader (TMV; Gallie, MOLECULAR BIOLOGY OF RNA, 237-56 (1989)); and maize chlorotic mottle virus leader (MCMV; Lommel et al., Virology 81, 382 (1991)). See also, Della-Cioppa et al., Plant Physiology 84, 965 (1987).

### III. NUCLEOTIDE SEQUENCES OF INTEREST

The heterologous nucleotide sequence(s) in the expression cassette can be any nucleotide sequence(s) of interest and can be obtained from prokaryotes or eukaryotes (e.g., bacteria, fungi, yeast, viruses, plants, mammals) or the heterologous nucleotide sequence can be synthesized in whole or in part. Further, the heterologous nucleotide sequence can encode a polypeptide of interest or can be transcribed to produce a functional RNA. In particular embodiments, the functional RNA can be expressed to improve an agronomic trait in the plant (e.g., tolerance to drought, heat stress, high temperature, salt, or resistance to herbicides disease, insects or other pests [e.g., a Bacillus thuringiensis endotoxin], and the like), to confer male sterility, to improve fertility and/or enhance nutritional quality (e.g., enzymes that enhance nutritional quality). A polypeptide of interest can be any polypeptide encoded by a nucleotide sequence of interest. The nucleotide sequence may further be used in the sense orientation to achieve suppression of endogenous plant genes, as is known by those skilled in the art (see, e.g., U.S. Pat. Nos. 5,283,184; 5,034,323).

The heterologous nucleotide sequence can encode a polypeptide that imparts a desirable agronomic trait to the plant (as described above), confers male sterility, improves fertility and/or improves nutritional quality. Other suitable polypeptides include enzymes that can degrade organic pollutants or remove heavy metals. Such plants, and the enzymes that can be isolated therefrom, are useful in methods of environmental protection and remediation. Alternatively, the heterologous nucleotide sequence can encode a therapeutically or pharmaceutically useful polypeptide or an industrial polypeptide (e.g., an industrial enzyme). Therapeutic polypeptides include, but are not limited to antibodies and antibody fragments, cytokines, hormones, growth factors, receptors, enzymes and the like.

Other non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, and/or fungal resistance. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956. See also, on the World Wide Web at lifesci.sussex.ac.uldhome/Neil_Crickmore/Bt/.

Nucleotide sequences conferring resistance/tolerance to an herbicide that inhibits the growing point or meristem, such as an imidazalinone or a sulfonylurea can also be suitable in some embodiments of the invention. Exemplary nucleotide sequences in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

In embodiments of the invention, the nucleotide sequence increases tolerance of a plant, plant part and/or plant cell to heat stress and/or high temperature. The nucleotide sequence can encode a polypeptide or inhibitory polynucleotide (e.g., functional RNA) that results in increased tolerance to heat stress and/or high temperature. Suitable polypeptides include without limitation water stress polypeptides, ABA receptors, dehydration proteins (e.g., ERDs), a glutamine synthetase 1;2 (GS1;2), a glutamate decarboxylase 3 (GAD3) and/or a class I glutamine amidotransferase (GAT1).

In representative embodiments, nucleotide sequences that encode polypeptides that provide tolerance to water stress are used. Non-limiting examples of polypeptides that provide tolerance to water stress include: water channel proteins involved in the movement of water through membranes; enzymes required for the biosynthesis of various osmoprotectants (e.g., sugars, proline, and Glycine-betaine); proteins that protect macromolecules and membranes (e.g., LEA protein, osmotin, antifreeze protein, chaperone and mRNA binding proteins); proteases for protein turnover (thiol proteases, CIp protease and ubiquitin); and detoxification enzymes (e.g., glutathione S-transferase, soluble epoxide hydrolase, catalase, superoxide dismutase and ascorbate peroxidase). Non-limiting examples of proteins involved in the regulation of signal transduction and gene expression in response to water stress include protein kinases (MAPK, MAPKKK, S6K, CDPK, two-component His kinase, Bacterial-type sensory kinase and SNF1); transcription factors (e.g., MYC and bZIP); phosopholipase C; and 14-3-3 proteins.

Nucleotide sequences that encode receptors/binding proteins for abscisic acid (ABA) are also useful in the practice of the present invention. Non-limiting examples of ABA binding proteins/receptors include: the Mg-chelatase H subunit; RNA-binding protein FCA; G-protein coupled receptor GCR2; PYR1; PYL5; protein phosphatases 2C ABI1 and ABI2; and proteins of the RCAR (Regulatory Component of the ABA Receptor) family.

In embodiments of the invention, the nucleotide sequence encodes a dehydration protein, also known as a dehydrin (e.g., an ERD). Dehydration proteins are a group of proteins known to accumulate in plants in response to dehydration. Examples include WCOR410 from wheat; PCA60 from peach; DHN3 from sessile oak, COR47 from Arabidopsis thaliana; Hsp90, BN59, BN115 and Bnerd10 from Brassica napes; COR39 and WCS19 from Triticum aestivum (bread wheat); and COR25 from Brassica rapa subsp. Pekinensis. Other examples of dehydration proteins are ERD proteins, which include without limitation, ERD1, ERD2, ERD4, ERD5, ERD6, ERD8, ERD10, ERD11, ERD13, ERD15 and ERD16.

Nucleic acids encoding a GS1;2 (E.C. 6.3.1.2), a GAD3 (E.C. 4.1.1.15), a GAT1 (E.C. 2.6.5.2), or any combination thereof can be used according to the invention. In representative embodiments, the GS1;2, GAD3 and/or GAT1 are plant enzymes. The GS1;2, GAD3 and GAT1 can be from any species of origin (e.g., rice [including indica and/or japonica varieties], wheat, barley, maize, sorghum, oats, rye, sugar cane, Arabidopsis and the like), and the terms “GS1;2,” “GAD3” and “GAT1” also include naturally occurring allelic variations, isoforms, splice variants and the like. The GS1;2, GAD3 and GAT1 can further be wholly or partially synthetic. These enzymes are well-known in the art and have previously been described in a number of plant species.

For example, it is known that plants have multiple isozymes of class 2 glutamine synthetase. The GS1:2 isozyme is a cytosolic form, and is involved in converting glutamine into glutamic acid and represents one of the early steps in amino acids biosynthesis. The enzyme is a homo-octomer composed of eight identical subunits separated into two face-to-face rings. ATP binds to the top of the active site near a cation binding site, whereas glutamate binds near a second cation binding site at the bottom of the active site. Ammonium, rather than ammonia, binds to active site because the binding site is polar and exposed to solvent. The nucleotide and amino acid sequences of a number of GS1;2 are known, e.g., in rice (GenBank Accession Nos. NP_{—}001051067 and P14654 [amino acid] and AB180688.1 and NM_{—}001057602 [nucleotide]), maize (GenBank Accession Nos. NP_{—}001105443 and BAA03433 [amino acid] and NM_{—}001111973 (nucleotide), soybean (GenBank Accession Nos. NP_{—}001242332 [amino acid] and NM_{—}001255403 [nucleotide]), Arabidopsis (GenBank Accession Nos. NP_{—}176794 [amino acid] and NM_{—}105291 [nucleotide]), sugar cane (GenBank Accession Nos. AAW21275 [amino acid] and AY835455 [nucleotide]), and the like. A number of functional domains have been identified in GS1;2 proteins including the beta-Grasp domain and catalytic domain. The crystal structure of the maize GS1a is described in Unno et al. (2006, J. Biol. Chem. 281:29287-29296; see also, RCSB Protein Data Bank ID 2D3C).

GAT1 is also known as carbamoyl phosphate synthetase and is involved in the first committed step in arginine biosynthesis in prokaryotes and eukaryotes. The nucleotide and amino acid sequences of a number of GAT1 are known, e.g., in rice (GenBank Accession Nos. BAD08105.1 and NP_OQ1047880 [amino acid] and NM_{—}001054415 [nucleotide]), maize (GenBank Accession Nos. NP_{—}001132055 [amino acid] and NM_{—}001138583 [nucleotide]), soybean (GenBank Accession Nos. XP_{—}003525104 [amino acid] and XM_{—}003525056 [nucleotide]), Arabidopsis (GenBank Accession Nos. NP_{—}566824 [amino acid] and NM_{—}113690 [nucleotide]), and the like. A number of functional domains have been identified in GAT1 proteins including the catalytic (active) site, which is defined by a conserved catalytic triad of cysteine, histidine and glutamate. The crystal structure of GAT1 from a number of bacteria have been described including the GAT1 From T. thermophilus (RCSB Protein Data Bank ID 2YWD) and P. horikoshii (RCSB Protein Data Bank ID 2D7J).

GAD3 is involved in converting L-glutamic acid into GABA. The nucleotide and amino acid sequences of a number of GAD3 are known, e.g., in rice (GenBank Accession Nos. AA059316 [amino acid] and AY187941 [nucleotide]), soybean (GenBank Accession Nos. BAF80895 [amino acid] and AB240965 [nucleotide]), Arabidopsis (GenBank Accession Nos. NP_{—}178309 [amino acid] and NM_{—}126261 [nucleotide]), and the like. A number of functional domains have been identified in GAD3 proteins including the pyridoxal 5′-phosphate (cofactor) binding site and the catalytic (active) site. The crystal structure of GAD3 has been resolved from bacteria, including E. coli (RCSB Protein Data Bank ID 3FZ7 and 3FZ6).

Polypeptides encoded by nucleotide sequences conferring resistance to glyphosate are also suitable for use with the present invention. See, e.g., U.S. Pat. No. 4,940,835 and U.S. Pat. No. 4,769,061. U.S. Pat. No. 5,554,798 discloses transgenic glyphosate resistant maize plants, which resistance is conferred by an altered 5-enolpyruvyl-3-phosphoshikimate (EPSP) synthase gene. Heterologous nucleotide sequences suitable to confer tolerance to the herbicide glyphosate also include, but are not limited to the Agrobacterium strain CP4 glyphosate resistant EPSPS gene (aroA:CP4) as described in U.S. Pat. No. 5,633,435 or the glyphosate oxidoreductase gene (GOX) as described in U.S. Pat. No. 5,463,175. Other heterologous nucleotide sequences include genes conferring resistance to herbicides that act to inhibit the action of acetolactate synthase (ALS), in particular the sulfonylurea-type herbicides (e.g., mutant forms of the acetolactate synthase (ALS) gene that lead to such resistance, in particular the S4 and/or Hra mutations), genes coding for resistance to herbicides that act to inhibit the action of glutamine synthase, such as phosphinothricin or basta (e.g., the bar gene). The bar gene encodes resistance to the herbicide basta, the nptlI gene encodes resistance to the antibiotics kanamycin and geneticin, and the ALS gene encodes resistance to the herbicide chlorsulfuron.

Nucleotide sequences coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268 and 5,561,236.

Other suitable nucleotide sequences include those coding for resistance to herbicides that inhibit photosynthesis, such as a triazine and a benzonitrile (nitrilase). See, U.S. Pat. No. 4,810,648. Additional suitable nucleotide sequences coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are nucleotide sequences conferring resistance to a protox enzyme, or that provide enhanced resistance to plant diseases; enhanced tolerance of adverse environmental conditions (abiotic stresses) including but not limited to drought, heat stress, high temperature, cold, excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Insecticidal proteins useful in the invention may be produced in an amount sufficient to control insect pests, i.e., insect controlling amounts. It is recognized that the amount of production of insecticidal protein in a plant useful to control insects may vary depending upon the cultivar, type of insect, environmental factors and the like. Suitable heterologous nucleotide sequences that confer insect tolerance include those which provide resistance to pests such as rootworm, cutworm, European Corn Borer, and the like. Exemplary nucleotide sequences include, but are not limited to, those that encode toxins identified in Bacillus organisms (see, e.g., WO 99/31248; U.S. Pat. Nos. 5,689,052; 5,500,365; 5,880,275); Bacillus thuringiensis toxic protein genes (see, e.g., U.S. Pat. Nos. 5,366,892; 5,747,450; 5,737,514; 5,723,756; 5,593,881; 6,555,655; 6,541,448; 6,538,109; Geiser, et al. (1986) Gene 48:109); and lectins (Van Damme et al. (1994) Plant Mol. Biol. 24:825). Nucleotide sequences encoding Bacillus thuringiensis (Bt) toxins from several subspecies have been cloned and recombinant clones have been found to be toxic to lepidopteran, dipteran and coleopteran insect larvae (for example, various delta-endotoxin genes such as Cry1Aa, Cry1Ab, Cry1Ac, Cry1B, Cry1C, Cry1D, Cry1Ea, Cry1Fa, Cry3A, Cry9A, Cry9C and Cry9B; as well as genes encoding vegetative insecticidal proteins such as Vip1, Vip2 and Vip3). A full list of Bt toxins can be found on the worldwide web at Bacillus thuringiensis Toxin Nomenclature Database maintained by the University of Sussex (see also, Crickmore et al. (1998) Microbiol. Mol. Biol. Rev. 62:807-813).

Polypeptides that are suitable for production in plants further include those that improve or otherwise facilitate the conversion of harvested plants and/or plant parts into a commercially useful product, including, for example, increased or altered carbohydrate content and/or distribution, improved fermentation properties, increased oil content, increased protein content, improved digestibility, and increased nutraceutical content, e.g., increased phytosterol content, increased tocopherol content, increased stanol content and/or increased vitamin content. Polypeptides of interest also include, for example, those resulting in, or contributing to, a reduced content of an unwanted component in a harvested crop, e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme).

In one embodiment, the polypeptide of interest contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced by xylanases. Heterologous production of xylanases in plant cells also can facilitate lignocellulosic conversion to fermentable sugars in industrial processing.

Numerous xylanases from fungal and bacterial microorganisms have been identified and characterized (see, e.g., U.S. Pat. No. 5,437,992; Coughlin at al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718).

In another embodiment, a polypeptide useful for the present invention can be a polysaccharide degrading enzyme. Plants producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme or other glucoamylases.

Polysaccharide-degrading enzymes include: starch degrading enzymes such as alpha-amylases (EC 3.2.1.1), glucuronidases (E.C. 3.2.1.131); exo-1,4-alpha-D glucanases such as amyloglucosidases and glucoamylase (EC 3.2.1.3), beta-amylases (EC 3.2.1.2), alpha-glucosidases (EC 3.2.1.20), and other exo-amylases; starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-beta-D-glucanase (EC 3.2.1.39), beta-glucosidase (EC 3.2.1.21); c) L-arabinases, such as endo-1,5-alpha-L-arabinase (EC 3.2.1.99), alpha-arabinosidases (EC 3.2.1.55) and the like; d) galactanases such as endo-1,4-beta-D-galactanase (EC 3.2.1.89), endo-1,3-beta-D-galactanase (EC 3.2.1.90), alpha-galactosidase (EC 3.2.1.22), beta-galactosidase (EC 3.2.1.23) and the like; e) mannanases, such as endo-1,4-beta-D-mannanase (EC 3.2.1.78), beta-mannosidase (EC 3.2.1.25), alpha-mannosidase (EC 3.2.1.24) and the like; f) xylanases, such as endo-1,4-beta-xylanase (EC 3.2.1.8), beta-D-xylosidase (EC 3.2.1.37), 1,3-beta-D-xylanase, and the like; and g) other enzymes such as alpha-L-fucosidase (EC 3.2.1.51), alpha-L-rhamnosidase (EC 3.2.1.40), levanase (EC 3.2.1.65), inulanase (EC 3.2.1.7), and the like.

Further enzymes which may be used with the present invention include proteases, such as fungal and bacterial proteases. Fungal proteases include, but are not limited to, those obtained from Aspergillus, Trichoderma, Mucor and Rhizopus, such as A. niger, A. awamori, A. oryzae and M. miehei. In some embodiments, the polypeptides of this invention can be cellobiohydrolase (CBH) enzymes (EC 3.2.1.91). In one embodiment, the cellobiohydrolase enzyme can be CBH1 or CBH2.

Other useful enzymes include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); esterases such as ferulic acid esterase (EC 3.1.1.73) and acetyl xylan esterases (EC 3.1.1.72); and cutinases (e.g. E.C. 3.1.1.74).

The nucleotide sequence can encode a reporter polypeptide (e.g., an enzyme), including but not limited to Green Fluorescent Protein, β-galactosidase, luciferase, alkaline phosphatase, the GUS gene encoding β-glucuronidase, and chloramphenicol acetyltransferase.

Where appropriate, the heterologous nucleotide sequence may be optimized for increased expression in a transformed plant, e.g., by using plant preferred codons. Methods for synthetic optimization of nucleic acid sequences are available in the art. The nucleotide sequence can be optimized for expression in a particular host plant or alternatively can be modified for optimal expression in monocots. See, e.g., EP 0 359 472, EP 0 385 962, WO 91/16432; Perlak et al., Prcc. Natl. Acad. Sol. USA 88, 3324 (1991), and Murray et al., Nuc. Acids Res. 17, 477 (1989), and the like. Plant preferred codons can be determined from the codons of highest frequency in the proteins expressed in that plant.

Additional sequence modifications are known to enhance gene expression in a cellular host. These include elimination of sequences encoding spurious polyadenylation signals, exon-intron splice site signals, transposon-like repeats, and other such well-characterized sequences which may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

### IV. TRANSGENIC PLANTS, PLANT PARTS AND PLANT CELLS

The invention also provides transgenic plants, plant parts and plant cells comprising the nucleic acids, expression cassettes and vectors of the invention.

Accordingly, as one aspect the invention provides a cell comprising a nucleic acid, expression cassette, or vector of the invention. The cell can be transiently or stably transformed with the nucleic acid, expression cassette or vector. Further, the cell can be a cultured cell, a cell obtained from a plant, plant part, or plant tissue, or a cell in situ in a plant, plant part or plant tissue. Cells can be from any suitable species, including plant (e.g. rice), bacterial, yeast, insect and/or mammalian cells. In representative embodiments, the cell is a plant cell or bacterial cell.

The invention also provides a plant part (including a plant tissue culture) comprising a nucleic acid, expression cassette, or vector of the invention. The plant part can be transiently or stably transformed with the nucleic acid, expression cassette or vector. Further, the plant part can be in culture, can be a plant part obtained from a plant, or a plant part in situ. In representative embodiments, the plant part comprises a cell of the invention.

Seed comprising the nucleic acid, expression cassette, or vector of the invention are also provided. Optionally, the nucleic acid, expression cassette or vector is stably incorporated into the genome of the seed.

The invention also contemplates a transgenic plant comprising a nucleic acid, expression cassette, or vector of the invention. The plant can be transiently or stably transformed with a nucleic acid, expression cassette or vector comprising a promoter sequence of the invention. In representative embodiments, the plant comprises a cell or plant part of the invention (as described above). In representative embodiments, the promoter sequence is inducible (e.g., has increased activity) in response to heat stress, high temperature, ABA, salicylic acid and/or MeJa.

Still further, the invention encompasses a crop comprising a plurality of the transgenic plants of the invention, as described herein. Nonlimiting examples of the types of crops comprising a plurality of transgenic plants of the invention include an agricultural field, a golf course, a residential lawn or garden, a public lawn or garden, a road side planting, an orchard, and/or a recreational field (e.g., a cultivated area comprising a plurality of the transgenic plants of the invention).

Products harvested from the plants of the invention are also provided. Nonlimiting examples of a harvested product include a seed, a leaf, a stem, a shoot, a fruit, flower, root, biomass (e.g., for biofuel production) and/or extract.

In some embodiments, a processed product produced from the harvested product is provided. Nonlimiting examples of a processed product include a polypeptide (e.g., a recombinant polypeptide), an extract, a medicinal product (e.g., artemicin as an antimalarial agent), a fiber or woven textile, a fragrance, dried fruit, a biofuel (e.g., ethanol), a tobacco product (e.g., cured tobacco, cigarettes, chewing tobacco, cigars, and the like), an oil (e.g., sunflower oil, corn oil, canola oil, and the like), a nut or seed butter, a flour or meal (e.g., wheat or rice flour, corn meal) and/or any other animal feed (e.g., soy, maize, barley, rice, alfalfa) and/or human food product (e.g., a processed wheat, maize, rice or soy food product).

### V. METHODS OF INTRODUCING NUCLEIC ACIDS

The invention also provides methods of introducing a nucleic acid, expression cassette or vector as described herein (e.g., SEQ ID NO:1, nucleotides 1921 to 4061 of SEQ ID NO:2, nucleotides 2562 to 4061 of SEQ ID NO:2, or SEQ ID NO:2 or equivalents thereof, including fragments) into a target plant, plant part or plant cell (including callus cells or protoplasts), seed, plant tissue (including callus), and the like. In exemplary embodiments, the method is practiced to express a nucleotide sequence of interest that is operably associated with a promoter as described herein. The invention further comprises host plants, cells, plant parts, seed or tissue culture (including callus) transiently or stably transformed with the nucleic acids, expression cassettes or vectors as described herein.

In representative embodiments, the invention provides a method of introducing a nucleotide sequence of interest into a plant, the method comprising transforming a plant cell with a nucleic acid, expression cassette, or vector comprising a promoter sequence as described herein to produce a transformed plant cell, and regenerating a stably transformed transgenic plant from the transformed plant cell.

In additional embodiments, the method comprises a method of expressing a nucleotide sequence of interest in a plant, the method comprising transforming a plant cell with an expression cassette or vector comprising a promoter sequence as described herein operably associated with a nucleotide sequence of interest to produce a transformed plant cell, regenerating a stably transformed transgenic plant from the transformed plant cell, and expressing the nucleotide sequence of interest in the plant.

Optionally, the methods of the invention can further comprise exposing the plant, plant part or plant cell to heat stress, high temperature, ABA, salicylic acid and/or MeJa.

The invention also provides a method of increasing tolerance of a plant, plant part or plant cell to heat stress or high temperature, the method comprising: transforming a plant, plant part or plant cell with an isolated nucleic acid, expression cassette or vector comprising a promoter sequence as described herein operably associated with a heterologous nucleotide sequence that provides increased tolerance to heat stress or high temperature (e.g., encodes a polypeptide that provides increased tolerance to heat stress or high temperature), including but not limited to, a coding sequence for a water stress polypeptide, an ABA receptor and/or a dehydration protein.

The invention further provides a method of increasing tolerance of a plant to heat stress or high temperature, the method comprising: (a) stably transforming a plant cell with an isolated nucleic acid, expression cassette, or vector comprising a promoter sequence as described herein operably associated with a heterologous nucleotide sequence that provides increased tolerance to heat stress or high temperature (e.g., encodes a polypeptide that provides increased tolerance to heat stress or high temperature), including but not limited to, a coding sequence for a water stress polypeptide, an ABA receptor and/or a dehydration protein; and (b) regenerating a stably transformed plant from the stably transformed plant cell of (a).

In representative embodiments, a method of increasing tolerance of a plant to heat stress or high temperature comprises reducing an adverse effect on plant functions, development and/or performance as a result of heat stress or high temperature, e.g., reduced cell division, size and/or number of plants and/or parts thereof and/or impairment in an agronomic trait such as reduced yield, fruit drop, fruit size and/or number, seed size and/or number, quality of produce due to appearance and/or texture and/or increased flower abortion.

The present invention can be advantageously practiced to regulate and/or increase the expression of a nucleotide sequence of interest operably associated with a promoter as described herein.

Accordingly, in embodiments, the invention provides a method of modulating (e.g., increasing) the expression of a nucleotide sequence of interest in response to heat stress or high temperature, ABA, salicylic acid and/or MeJa, the method comprising transforming a plant, plant part or plant cell with a nucleic acid, expression cassette or vector as described herein, and optionally, exposing the plant, plant part or plant cell to heat stress or high temperature, ABA, salicylic acid and/or MeJa.

In additional embodiments, the invention provides a method of modulating (e.g., increasing) the expression of a nucleotide sequence of interest in response to heat stress or high temperature, ABA, salicylic acid and/or MeJa, the method comprising (a) stably transforming a plant cell with an isolated nucleic acid, expression cassette or vector as described herein; (b) regenerating a stably transformed plant from the stably transformed plant cell of (a); and (c) exposing the plant to heat stress or high temperature, ABA, salicylic acid and/or MeJa.

The invention further encompasses transgenic plants (and progeny thereof), plant parts, and plant cells produced by the methods of the invention.

Also provided by the invention are seed produced from the inventive transgenic plants. Optionally, the seed comprise a nucleic acid, expression cassette or vector as described herein stably incorporated into the genome.

Methods of introducing nucleic acids, transiently or stably, into plants, plant tissues, cells, protoplasts, seed, callus and the like are known in the art. Stably transformed nucleic acids can be incorporated into the genome. Exemplary transformation methods include biological methods using viruses and bacteria (e.g., Agrobacterium), physicochemical methods such as electroporation, floral dip methods, ballistic bombardment, microinjection, and the like. Other transformation technology includes the whiskers technology that is based on mineral fibers (see e.g., U.S. Pat. Nos. 5,302,523 and 5,464,765) and pollen tube transformation.

Other exemplary transformation methods include, without limitation, calcium-phosphate-mediated transformation, cyclodextrin-mediated transformation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Thus, in some particular embodiments, the method of introducing into a plant, plant part, plant tissue, plant cell, protoplast, seed, callus and the like comprises bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethyleneglycol-mediated transformation, any other electrical, chemical, physical and/or biological mechanism that results in the introduction of nucleic acid into the plant, plant part and/or cell thereof, or a combination thereof.

In one form of direct transformation, the vector is microinjected directly into plant cells by use of micropipettes to mechanically transfer the recombinant DNA (Crossway, Mol. Gen. Genetics 202: 179 (1985)).

In another protocol, the genetic material is transferred into the plant cell using polyethylene glycol (Krens, et al. Nature 296, 72 (1982)).

In still another method, protoplasts are fused with minicells, cells, lysosomes, or other fusible lipid-surfaced bodies that contain the nucleotide sequence to be transferred to the plant (Fraley, et al., Proc. Natl. Acad. Sci. USA 79, 1859 (1982)).

Nucleic acids may also be introduced into the plant cells by electroporation (Fromm et al., Proc. Natl. Acad. Sci. USA 82, 5824 (1985)). In this technique, plant protoplasts are electroporated in the presence of nucleic acids comprising the expression cassette. Electrical impulses of high field strength reversibly permeabilize biomembranes allowing the introduction of the nucleic acid. Electroporated plant protoplasts reform the cell wall, divide and regenerate. One advantage of electroporation is that large pieces of DNA, including artificial chromosomes, can be transformed by this method.

Ballistic transformation typically comprises the steps of: (a) providing a plant material as a target; (b) propelling a microprojectile carrying the heterologous nucleotide sequence at the plant target at a velocity sufficient to pierce the walls of the cells within the target and to deposit the nucleotide sequence within a cell of the target to thereby provide a transformed target. The method can further include the step of culturing the transformed target with a selection agent and, optionally, regeneration of a transformed plant. As noted below, the technique may be carried out with the nucleotide sequence as a precipitate (wet or freeze-dried) alone, in place of the aqueous solution containing the nucleotide sequence.

Any ballistic cell transformation apparatus can be used in practicing the present invention. Exemplary apparatus are disclosed by Sandford et al. (Particulate Science and Technology 5, 27 (1988)), Klein et al. (Nature 327, 70 (1987)), and in EP 0 270 356. Such apparatus have been used to transform maize cells (Klein et aL, Proc. Natl. Acad. Sci. USA 85, 4305 (1988)), soybean callus (Christou et al., Plant Physiol. 87, 671 (1988)), McCabe et al., BioTechnology 6, 923 (1988), yeast mitochondria (Johnston et al., Science 240, 1538 (1988)), and Chlamydomonas chloroplasts (Boynton et al., Science 240, 1534 (1988)).

Alternately, an apparatus configured as described by Klein et al. (Nature 70, 327 (1987)) may be utilized. This apparatus comprises a bombardment chamber, which is divided into two separate compartments by an adjustable-height stopping plate. An acceleration tube is mounted on top of the bombardment chamber. A macroprojectile is propelled down the acceleration tube at the stopping plate by a gunpowder charge. The stopping plate has a borehole formed therein, which is smaller in diameter than the microprojectile. The macroprojectile carries the microprojectile(s), and the macroprojectile is aimed and fired at the borehole. When the macroprojectile is stopped by the stopping plate, the microprojectile(s) is propelled through the borehole. The target is positioned in the bombardment chamber so that a microprojectile(s) propelled through the bore hole penetrates the cell walls of the cells in the target and deposit the nucleotide sequence of interest carried thereon in the cells of the target. The bombardment chamber is partially evacuated prior to use to prevent atmospheric drag from unduly slowing the microprojectiles. The chamber is only partially evacuated so that the target tissue is not desiccated during bombardment. A vacuum of between about 400 to about 800 millimeters of mercury is suitable.

In alternate embodiments, ballistic transformation is achieved without use of microprojectiles. For example, an aqueous solution containing the nucleotide sequence of interest as a precipitate may be carried by the macroprojectile (e.g., by placing the aqueous solution directly on the plate-contact end of the macroprojectile without a microprojectile, where it is held by surface tension), and the solution alone propelled at the plant tissue target (e.g., by propelling the macroprojectile down the acceleration tube in the same manner as described above). Other approaches include placing the nucleic acid precipitate itself (“wet” precipitate) or a freeze-dried nucleotide precipitate directly on the plate-contact end of the macroprojectile without a microprojectile. In the absence of a microprojectile, it is believed that the nucleotide sequence must either be propelled at the tissue target at a greater velocity than that needed if carried by a microprojectile, or the nucleotide sequenced caused to travel a shorter distance to the target (or both).

It particular embodiments, the nucleotide sequence is delivered by a microprojectile. The microprojectile can be formed from any material having sufficient density and cohesiveness to be propelled through the cell wall, given the particle's velocity and the distance the particle must travel. Non-limiting examples of materials for making microprojectiles include metal, glass, silica, ice, polyethylene, polypropylene, polycarbonate, and carbon compounds (e.g., graphite, diamond). Non-limiting examples of suitable metals include tungsten, gold, and iridium. The particles should be of a size sufficiently small to avoid excessive disruption of the cells they contact in the target tissue, and sufficiently large to provide the inertia required to penetrate to the cell of interest in the target tissue. Particles ranging in diameter from about one-half micrometer to about three micrometers are suitable. Particles need not be spherical, as surface irregularities on the particles may enhance their carrying capacity.

The nucleotide sequence may be immobilized on the particle by precipitation. The precise precipitation parameters employed will vary depending upon factors such as the particle acceleration procedure employed, as is known in the art. The carrier particles may optionally be coated with an encapsulating agents such as polylysine to improve the stability of nucleotide sequences immobilized thereon, as discussed in EP 0 270 356 (column 8).

Alternatively, plants may be transformed using Agrobacterium tumefaciens or Agrobacterium rhizogenes. Agrobacterium-mediated nucleic acid transfer exploits the natural ability of A. tumefaciens and A. rhizogenes to transfer DNA into plant chromosomes. Agrobacterium is a plant pathogen that transfers a set of genes encoded in a region called T-DNA of the Ti and Ri plasmids of A. tumefaciens and A. rhizogenes, respectively, into plant cells. The typical result of transfer of the Ti plasmid is a tumorous growth called a crown gall in which the T-DNA is stably integrated into a host chromosome. Integration of the Ri plasmid into the host chromosomal DNA results in a condition known as “hairy root disease”. The ability to cause disease in the host plant can be removed by deletion of the genes in the T-DNA without loss of DNA transfer and integration. The DNA to be transferred is attached to border sequences that define the end points of an integrated T-DNA.

Transfer by means of engineered Agrobacterium strains has become routine for many dicotyledonous plants. Some difficulty has been experienced, however, in using Agrobacterium to transform monocotyledonous plants, in particular, cereal plants. However, Agrobacterium mediated transformation has been achieved in several monocot species, including cereal species such as rye, maize (Rhodes et al., Science 240, 204 (1988)), and rice (Hiei et al., (1994) Plant J. 6:271).

While the following discussion will focus on using A. tumefaciens to achieve gene transfer in plants, those skilled in the art will appreciate that this discussion also applies to A. rhizogenes. Transformation using A. rhizogenes has developed analogously to that of A. tumefaciens and has been successfully utilized to transform, for example, alfalfa, Solanum nigrum L., and poplar (U.S. Pat. No. 5,777,200 to Ryals et al.). As described by U.S. Pat. No. 5,773,693 to Burgess et al., it is preferable to use a disarmed A. tumefaciens strain (as described below), however, the wild-type A. rhizogenes may be employed. An illustrative strain of A. rhizogenes is strain 15834.

In particular protocols, the Agrobacterium strain is modified to contain the nucleotide sequences to be transferred to the plant. The nucleotide sequence to be transferred is incorporated into the T-region and is typically flanked by at least one T-DNA border sequence, optionally two T-DNA border sequences. A variety of Agrobacterium strains are known in the art particularly, and can be used in the methods of the invention. See, e.g., Hooykaas, Plant Mol. Biol. 13, 327 (1989); Smith et al., Crop Science 35, 301 (1995); Chilton, Proc. Natl. Acad. Sci. USA 90, 3119 (1993); Mollony et al., Monograph Theor. Appl. Genet NY 19, 148 (1993); Ishida et al., Nature Biotechnol. 14, 745 (1996); and Komari et al., The Plant Journal 10, 165 (1996).

In addition to the T-region, the Ti (or Ri) plasmid contains a vir region. The vir region is important for efficient transformation, and appears to be species-specific.

Two exemplary classes of recombinant Ti and Ri plasmid vector systems are commonly used in the art. In one class, called “cointegrate,” the shuttle vector containing the gene of interest is inserted by genetic recombination into a non-oncogenic Ti plasmid that contains both the cis-acting and trans-acting elements required for plant transformation as, for example, in the PMLJ1 shuttle vector of DeBlock et al., EMBO J 3, 1681 (1984), and the non-oncogenic Ti plasmid pGV2850 described by Zambryski et al., EMBOJ 2, 2143 (1983). In the second class or “binary” system, the gene of interest is inserted into a shuttle vector containing the cis-acting elements required for plant transformation. The other necessary functions are provided in trans by the non-oncogenic Ti plasmid as exemplified by the pBIN19 shuttle vector described by Bevan, Nucleic Acids Research 12, 8711 (1984), and the non-oncogenic Ti plasmid PAL4404 described by Hoekma, et al., Nature 303, 179 (1983).

Binary vector systems have been developed where the manipulated disarmed T-DNA carrying the heterologous nucleotide sequence of interest and the vir functions are present on separate plasmids. In this manner, a modified T-DNA region comprising foreign DNA (the nucleic acid to be transferred) is constructed in a small plasmid that replicates in E. coli. This plasmid is transferred conjugatively in a tri-parental mating or via electroporation into A. tumefaciens that contains a compatible plasmid with virulence gene sequences. The vir functions are supplied in trans to transfer the T-DNA into the plant genome. Such binary vectors are useful in the practice of the present invention.

In particular embodiments of the invention, super-binary vectors are employed. See, e.g., U.S. Pat. No. 5,591,615 and EP 0 604 662. Such a super-binary vector has been constructed containing a DNA region originating from the hypervirulence region of the Ti plasmid pTiBo542 (Jin et al., J. Bacteriol. 169, 4417 (1987)) contained in a super-virulent A. tumefaciens A281 exhibiting extremely high transformation efficiency (Hood et al., Biotechnol. 2, 702 (1984); Hood et al., J. Bacteriol. 168, 1283 (1986); Komari et al., J. Bacteriol. 166, 88 (1986); Jin et al., J. Bacteriol. 169, 4417 (1987); Komari, Plant Science 60, 223 (1987); ATCO Accession No. 37394.

Exemplary super-binary vectors known to those skilled in the art include pTOK162 (Japanese patent Appl. (Kokai) No. 4-222527, EP 504,869, EP 604,662, and U.S. Pat. No. 5,591,616) and pTOK233 (Komari, Plant Cell Reports 9, 303 (1990); Ishida et al., Nature Biotechnology 14, 745 (1996)). Other super-binary vectors may be constructed by the methods set forth in the above references. Super-binary vector pTOK162 is capable of replication in both E. coli and in A. tumefaciens. Additionally, the vector contains the virB, virC and virG genes from the virulence region of pTiBo542. The plasmid also contains an antibiotic resistance gene, a selectable marker gene, and the nucleic acid of interest to be transformed into the plant. The nucleic acid to be inserted into the plant genome is typically located between the two border sequences of the T region. Super-binary vectors of the invention can be constructed having the features described above for pTOK162. The T-region of the super-binary vectors and other vectors for use in the invention are constructed to have restriction sites for the insertion of the genes to be delivered. Alternatively, the DNA to be transformed can be inserted in the T-DNA region of the vector by utilizing in vivo homologous recombination. See, Herrera-Esterella et al., EMBO J. 2, 987 (1983); Horch et al., Science 223, 496 (1984). Such homologous recombination relies on the fact that the super-binary vector has a region homologous with a region of pBR322 or other similar plasmids. Thus, when the two plasmids are brought together, a desired gene is inserted into the super-binary vector by genetic recombination via the homologous regions.

In plants stably transformed by Agrobacteria-mediated transformation, the nucleotide sequence of interest is incorporated into the plant nuclear genome, typically flanked by at least one T-DNA border sequence and generally two T-DNA border sequences.

Plant cells may be transformed with Agrobacteria by any means known in the art, e.g., by co-cultivation with cultured isolated protoplasts, or transformation of intact cells or tissues. The first uses an established culture system that allows for culturing protoplasts and subsequent plant regeneration from cultured protoplasts. Identification of transformed cells or plants is generally accomplished by including a selectable marker in the transforming vector, or by obtaining evidence of successful bacterial infection.

Methods of introducing a nucleic acid into a plant can also comprise in vivo modification of genetic material, methods for which are known in the art. For example, in vivo modification can be used to insert a nucleic acid comprising a promoter sequence of the invention into the plant genome.

Suitable methods for in vivo modification include the techniques described in Gao et. al., Plant J. 61, 176 (2010); Li et al., Nucleic Acids Res. 39, 359 (2011); U.S. Pat. Nos. 7,897,372 and 8,021,867; U.S. Patent Publication No. 2011/0145940 and in International Patent Publication Nos. WO 2009/114321, WO 2009/134714 and WO 2010/079430. For example, one or more transcription affector-like nucleases (TALEN) and/or one or more meganucleases may be used to incorporate an isolated nucleic acid comprising a promoter sequence of the invention into the plant genome. In representative embodiments, the method comprises cleaving the plant genome at a target site with a TALEN and/or a meganuclease and providing a nucleic acid that is homologous to at least a portion of the target site and further comprises a promoter sequence of the invention (optionally in operable association with a heterologous nucleotide sequence of interest), such that homologous recombination occurs and results in the insertion of the promoter sequence of the invention into the genome.

Protoplasts, which have been transformed by any method known in the art, can also be regenerated to produce intact plants using known techniques.

Plant regeneration from cultured protoplasts is described in Evans et al., Handbook of Plant Cell Cultures, Vol. 1: (MacMilan Publishing Co. New York, 1983); and Vasil I. R. (ed.), Cell Culture and Somatic Cell Genetics of Plants, Acad. Press, Orlando, Vol. I, 1984, and Vol. II, 1986). Essentially all plant species can be regenerated from cultured cells or tissues, including but not limited to, all major species of sugar-cane, sugar beet, cotton, fruit trees, and legumes.

Means for regeneration vary from species to species of plants, but generally a suspension of transformed protoplasts or a petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

The regenerated plants are transferred to standard soil conditions and cultivated in a conventional manner. The plants are grown and harvested using conventional procedures.

Alternatively, transgenic plants may be produced using the floral dip method (See, e.g., Clough and Bent (1998) Plant Journal 16:735-743, which avoids the need for plant tissue culture or regeneration. In one representative protocol, plants are grown in soil until the primary inflorescence is about 10 cm tall. The primary inflorescence is cut to induce the emergence of multiple secondary inflorescences. The inflorescences of these plants are typically dipped in a suspension of Agrobacterium containing the vector of interest, a simple sugar (e.g., sucrose) and surfactant. After the dipping process, the plants are grown to maturity and the seeds are harvested. Transgenic seeds from these treated plants can be selected by germination under selective pressure (e.g., using the chemical bialaphos). Transgenic plants containing the selectable marker survive treatment and can be transplanted to individual pots for subsequent analysis. See Bechtold, N. and Pelletier, G. Methods Mol Biol 82, 259-266 (1998); Chung, M. H. et al. Transgenic Res 9, 471-476 (2000); Clough, S. J. and Bent, A. F. Plant J 16, 735-743 (1998); Mysore, K. S. et al. Plant J 21, 9-16 (2000); Tague, B. W. Transgenic Res 10, 259-267 (2001); Wang, W. C. et al. Plant Cell Rep 22, 274-281 (2003); Ye, Q. N. et al. Plant J., 19:249-257 (1999).

The particular conditions for transformation, selection and regeneration can be optimized by those of skill in the art. Factors that affect the efficiency of transformation include the species of plant, the target tissue or cell, composition of the culture media, selectable marker genes, kinds of vectors, and light/dark conditions. Therefore, these and other factors may be varied to determine what is an optimal transformation protocol for any particular plant species. It is recognized that not every species will react in the same manner to the transformation conditions and may require a slightly different modification of the protocols disclosed herein. However, by altering each of the variables, an optimum protocol can be derived for any plant species.

Further, the genetic properties engineered into the transgenic seeds and plants, plant parts, and/or plant cells of the present invention described herein can be passed on by sexual reproduction or vegetative growth and therefore can be maintained and propagated in progeny plants. Generally, maintenance and propagation make use of known agricultural methods developed to fit specific purposes such as harvesting, sowing or tilling.

The present invention is more particularly described in the following examples that are intended as illustrative only since numerous modifications and variations therein will be apparent to those skilled in the art.

### EXAMPLES

As used in the following Examples, the term “normal growth conditions” refers to growth conditions comprising 29° C. daytime temperatures, 23° C. nighttime temperatures, 12 hours of light (approximately 500 μmol m⁻² s⁻¹) during the daytime and 12 hours of dark during the nighttime.

As used in the following Examples, the term “normal temperature conditions” refers to growth conditions comprising 29° C. daytime temperatures and 23° C. nighttime temperatures.

As used in the following Examples, the term “high temperature conditions” refers to growth conditions comprising 35° C. daytime temperatures and 26° C. nighttime temperatures.

As used in the following Examples, the term “normal daylight conditions” refers to growth conditions comprising 12 hours of light (approximately 500 μmol m⁻² s⁻¹) during the daytime and 12 hours of dark during the nighttime.

As used in the following Examples, the term “long daylight conditions” refers to growth conditions comprising 16 hours of light (approximately 500 μmol m⁻² s⁻¹) during the daytime and 8 hours of dark during the nighttime.

### Example 1

### Characterization of OsMYB55

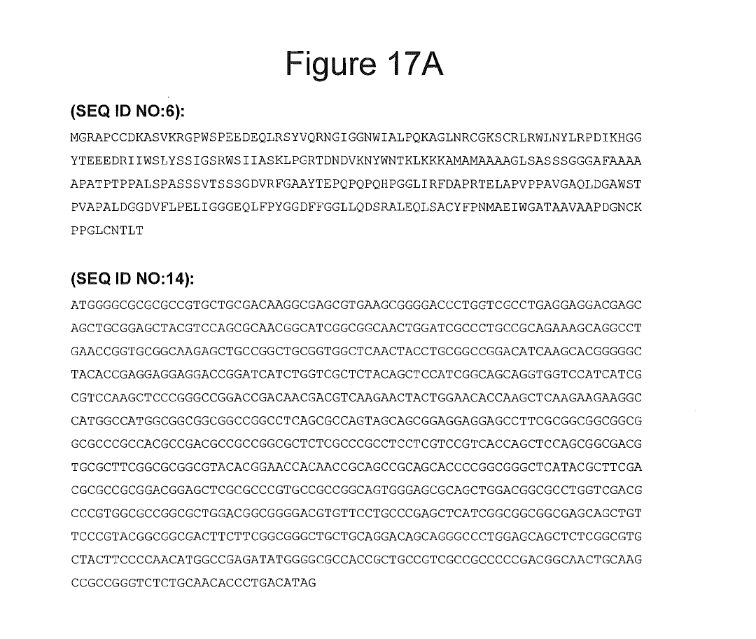

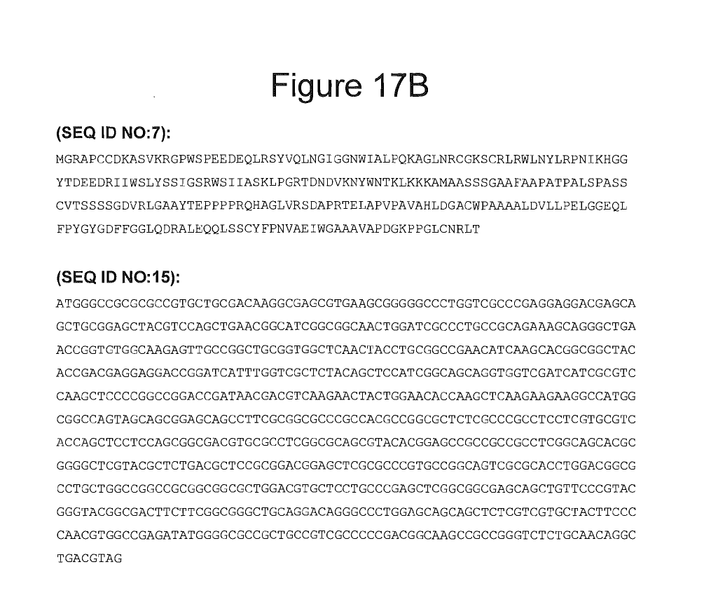

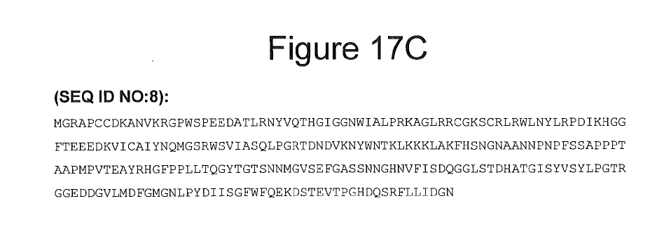

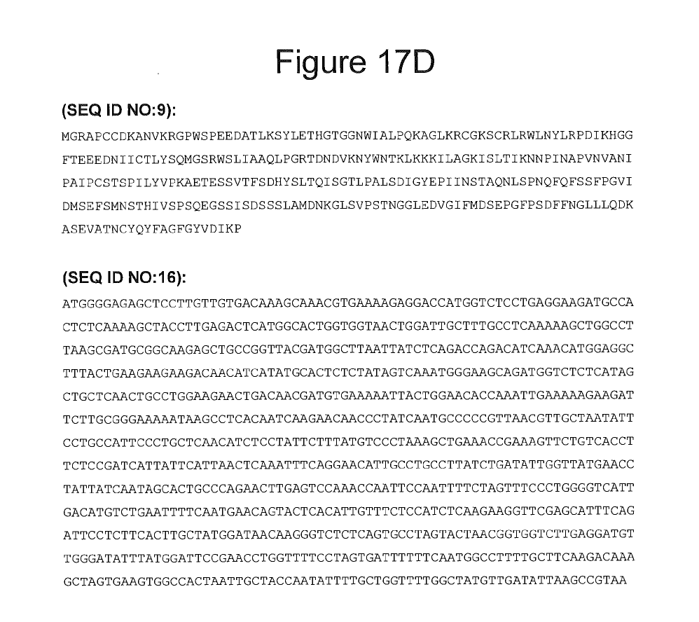

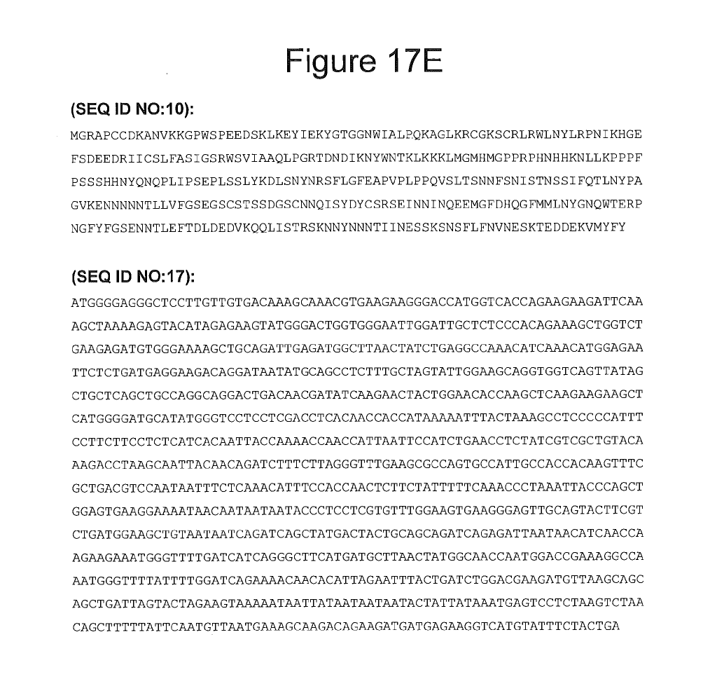

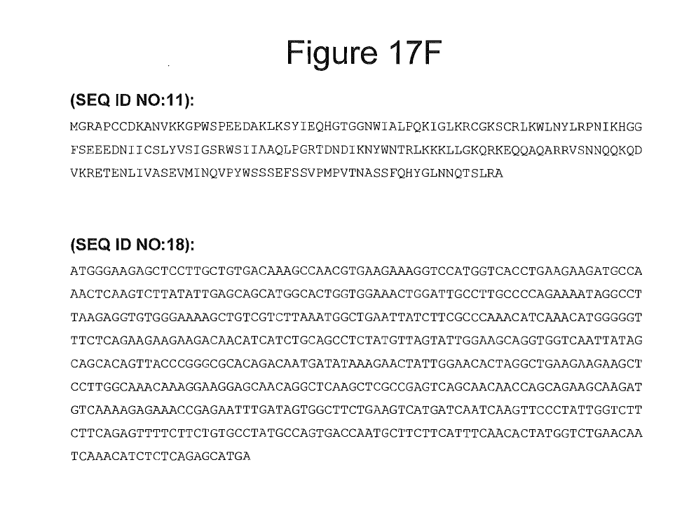

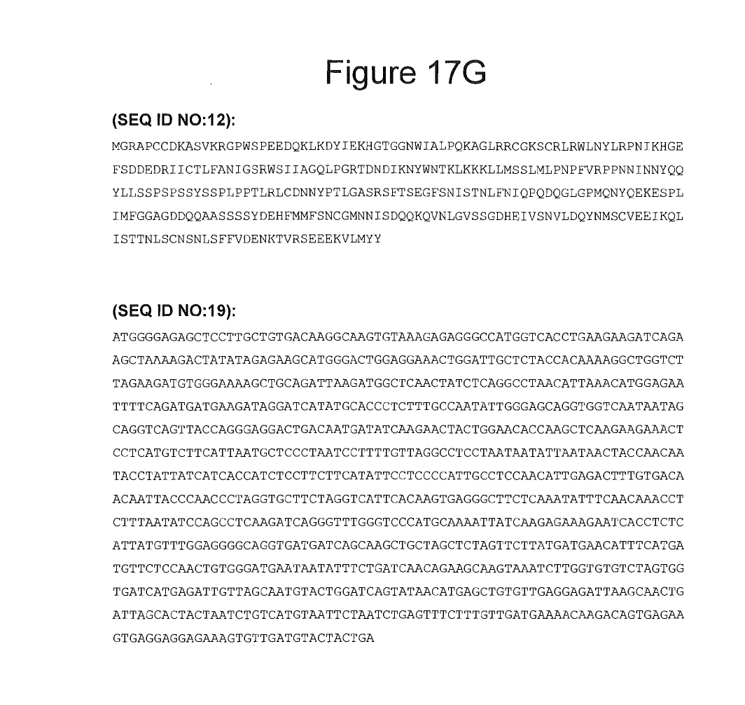

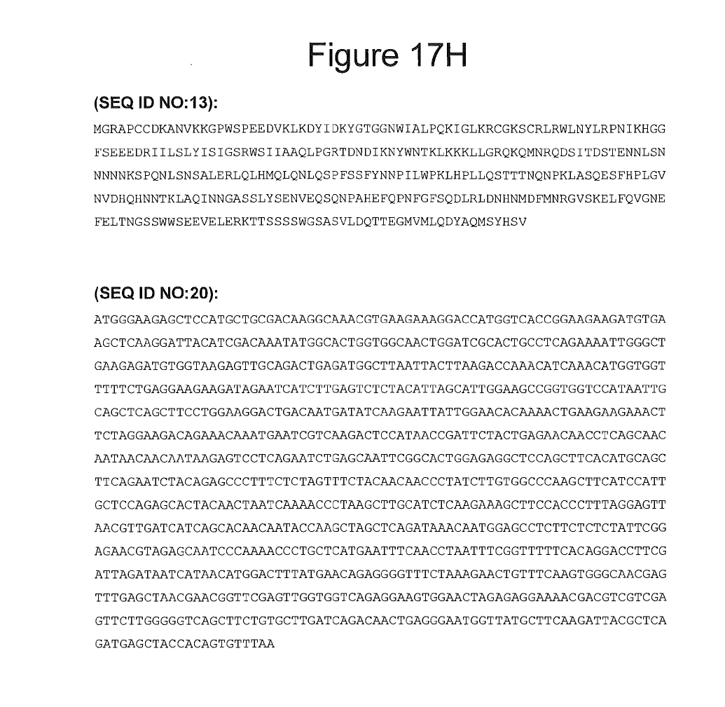

The 867 bp full length cDNA sequence (SEQ ID NO:4; FIG. 1E) of OsMYB55 encodes an R2R3-MYB transcription factor predicted to be 289 amino acids in length (SEQ ID NO:5; FIG. 1F). A BLAST® (National Center for Biotechnology Information, Bethesda, Md.) search was used to identify homologues of OsMYB55. Amino acid sequences of the closest homologs (SEQ ID NQs:6-13; FIGS. 17A-H) were used to generate a phylogenetic tree showing the similarity between OsMYB55 and its homologues (FIG. 1A).

The genomic DNA sequence containing the 5′UTR, promoter sequence, the MYB55 coding region (containing three exons and two introns) and 3′ UTR are shown in FIG. 1D (SEQ ID NO:3); the 5′ UTR and promoter sequence alone are shown in FIG. 1C (SEQ ID NO:2). A 2134 bp portion of the OsMYB55 promoter sequence (lacking nucleotides −1 to −5) is shown in FIG. 1B (SEQ ID NQ:1) and was used to construct a GUS reporter construct (Example 3).

To understand the regulation of the OsMYB55 gene, an in-silico analysis of the OsMYB55 promoter region (2100 bp) was carried out using the PlantCARE database (Flanders Interuniversity Institute for Biotechnology, Zwijnaarde, Belgium; available at http://bioinformatics.psb.ugent.be/webtools/plantcare/html/). Numerous potential CAREs and TFBS were identified in the OsMYB55 promoter region (FIGS. 2A-2B).

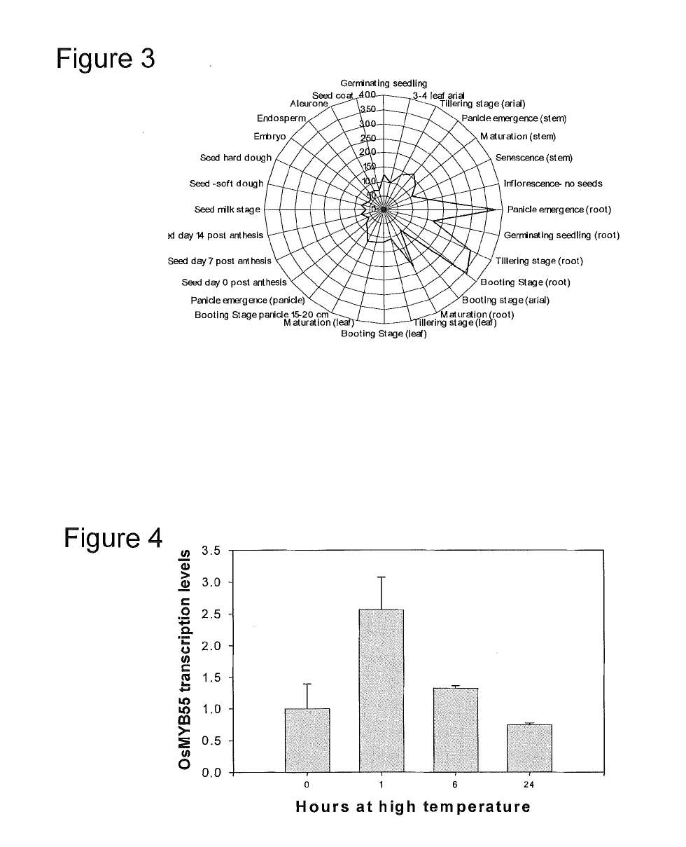

Global expression analysis revealed that OsMYB55 is differentially expressed in plant tissues and that its expression varies throughout the plant's life cycle (FIG. 3). Transcript levels were higher during vegetative stages up to tillering and the inflorescence stage. Transcription was higher in root tissues than in leaf tissues during those stages. OsMYB55's lowest expression level was observed in seeds, both during seed development at seed maturation.

### Example 2

### OsMYB55 Expression is Up-Regulated

in Response to Heat Stress:OsMYB55 Transcripts Seeds from wild-type rice plants were planted in 500 ml pots containing a growth media comprising peat moss and vermiculite in a ratio of 1:4. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions for four weeks.

Following four weeks of growth under normal growth conditions, plants were exposed to 45° C. for 0, 1, 6 or 24 hours. Leaves were harvested from each plant, frozen immediately in liquid nitrogen and stored at −80° C.

Quantitative real-time RT-PCR analysis of the leaves showed that OsMYB55 expression was up-regulated following exposure to 45° C. for one hour and that OsMY1355 expression returned to basal levels following exposure to 45° C. for 6 or 24 hours (FIG. 4).

### Example 3

### OsMYB55 Expression is Up-Regulated in Response to Heat Stress: GUS Reporter Protein

To generate the OsMYB55promoter-GUS construct, a 2134 base pair fragment of the OsMYB55 promoter region was amplified from genomic DNA using the OsMYB55promoter-BamH1 forward primer (5′-TGGTGAGGAGGATTGTGCAAGGATCCGCG-3′; SEQ ID NO:21) and the OsMYB55promoter-EcoR1 reverse primer (5′-CCGGAATTCTTGCACAATCCTCCT CACCA-3′; SEQ ID NO:22).

DNA was isolated from four-week-old plants grown under normal growth conditions using the cetyl trimethylammonium bromide (CTAB) extraction method. The amplified fragment (SEQ ID NO: 1; FIG. 1B) was cloned into the multiple cloning site of the pCAMBIA1391Z (Cambia, Brisbane, Australia) between the BamHI and EcoRI restriction sites to drive expression of the GUS reporter protein. Transgenic rice lines comprising the OsMYB55promoter-GUS construct were generated using Agrobacterium-mediated transformation, and positively transformed lines were selected according to the methods of Miki et al., PLANT PHYSIOL. 138(4):1903 (2005).

Seeds from transgenic rice plants expressing the OsMYB55promoter-GUS construct were planted in 500 ml pots containing a growth media comprising peat moss and vermiculite in a ratio of 1:4. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions for four weeks.

Following four weeks of growth under normal growth conditions, plants were exposed to 29° C. or 45° C. for 0, 1, 6 or 24 hours. Plant tissues were harvested 24 hours after treatment and stained by immersion in 0.1 M sodium citrate-HCl buffer pH 7.0 containing 1 mg/ml 5-bromo-4-chloro-3-indolyl-β-D-glucuronic acid (X-Gluc) (Biosynth International, Inc., Itasca, Ill.), vacuum infiltration for five minutes and incubation at 37° C. for 16 hours. Chlorophyll was removed from the tissues by incubating the tissues in 75% ethanol. The samples were conserved in glycerol 10% until examination. Hand sections were prepared and investigated using a light microscopy.

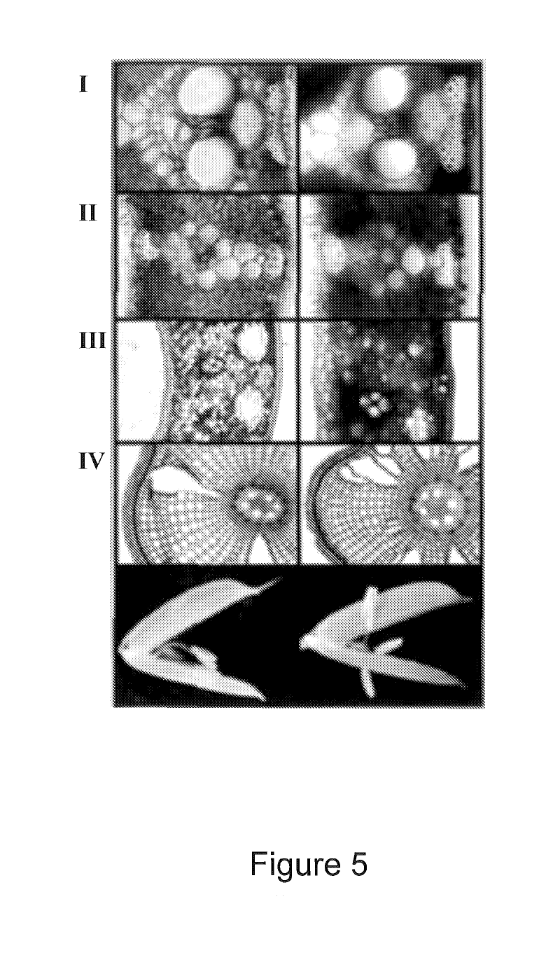

As shown in FIG. 5, GUS expression was higher in plants exposed to 45° C. for 24 hours than in plants exposed to 29° C. for 24 hours.

### Example 4

### Transgenic Rice Plants Overexpressing OsMYB55

Since OsMYB55 expression is up-regulated in response to high temperatures, we investigated whether OsMYB55 expression plays a role in one or more heat stress responses and/or heat tolerance. Experiments were carried out at different developmental stages to determine the effect of OsMYB55 overexpression on plant thermotolerance.

Constructs for over-expressing OsMYB55 were created using the maize ubiquitin promoter. Agrobacterium-mediated transformation was used to generate transgenic plants. Positively transformed plants were selected using the phosphomannose isomerase (PMI) test (Negrotto et al. PLANT CELL REP. 19:798 (2000)).

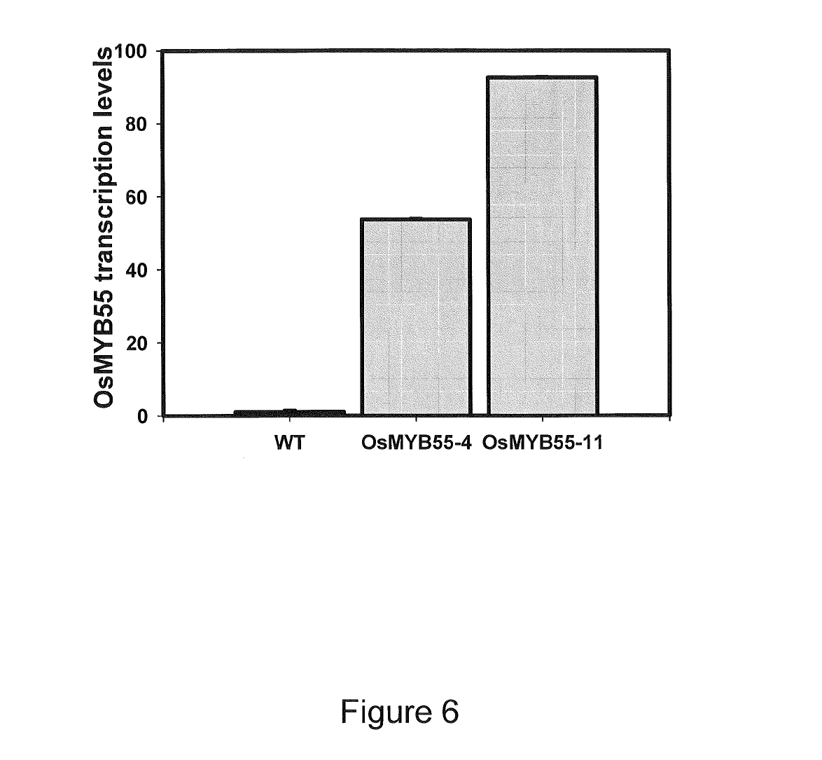

Expression analysis showed that OsMYB55 expression following four weeks of growth under normal growth conditions was fifty to ninety times higher in the leaves of transgenic rice plants overexpressing OsMYB55 as compared to wild-type rice plants (FIG. 6).

### Example 5

### OsMYB55 Overexpression Increases Coleoptile Length at High Temperatures

Seeds from wild-type rice plants and transgenic rice plants overexpressing OsMYB55 were germinated and grown for four days at 28° C. or 39° C.

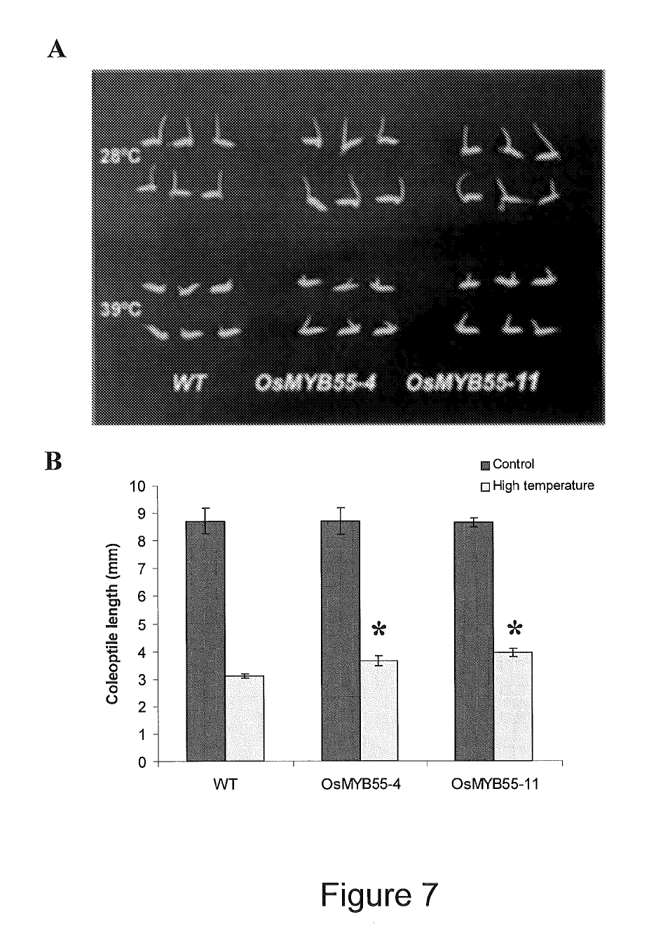

As shown in FIGS. 7A and 7B, although coleoptile length was reduced in all of the plants grown at 39° C., the coleoptiles of transgenic rice plants overexpressing OsMYB55 grown at 39° C. were significantly longer than those of their wild-type counterparts.

### Example 6

### OsMYB55 Overexpression Enhances Growth Under Long Daylight Conditions

Seeds from wild-type rice plants and transgenic rice plants overexpressing OsMYB55 were planted in 500 ml pots containing Turface® MVP® (PROFILE Products, LLC, Buffalo Grove, Ill.) (a 100% baked calcined clay growth media with grain size between 2.5 and 3.5 mm). Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for 4 weeks under long daylight conditions with either normal temperature conditions or high temperature conditions.

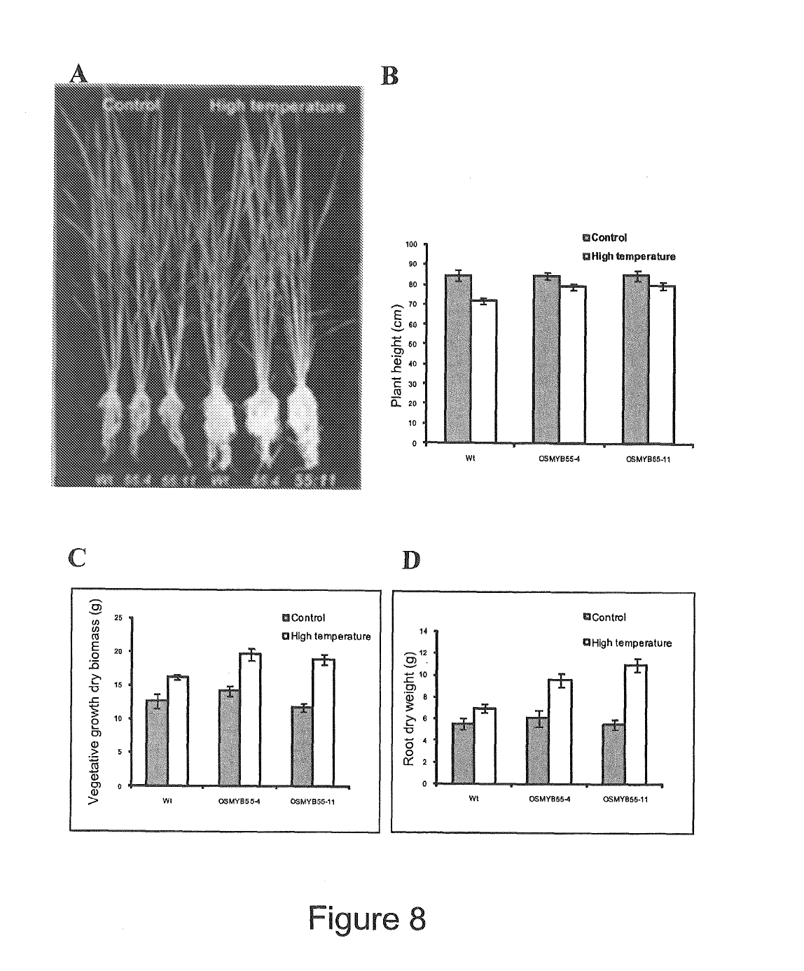

As shown in FIGS. 8A-8D, there were no significant differences in the heights, vegetative biomasses and root biomasses of plants grown under normal temperature conditions. Wild-type rice plants grown under high temperature conditions showed a decrease in plant height (FIG. 8B) and an increase in dry biomass (FIGS. 8C-8D). Transgenic rice plants overexpressing OsMYB55 grown under high temperature conditions showed less reduction in plant height (FIG. 8B) and a significant increase in plant biomass as compared to their wild-type counterparts (FIGS. 8C-8D).

### Example 7

### OsMYB55 Overexpression Enhances Growth Under Normal Daylight Conditions

Seeds from wild-type rice plants and transgenic rice plants overexpressing OsMYB55 were planted in 500 ml pots containing a growth media comprising peat moss and vermiculite in a ratio of 1:4. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under normal daylight conditions with either normal temperature conditions or high temperature conditions.

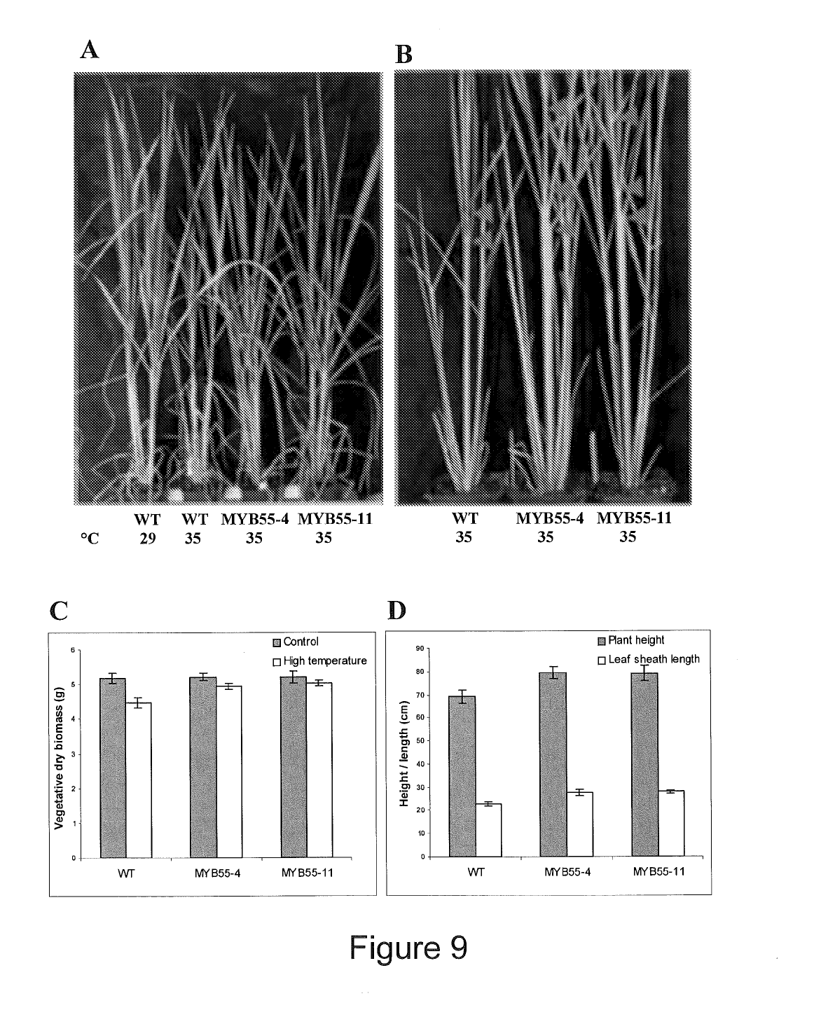

As shown in FIGS. 9A-9D, there were no significant differences in the heights and vegetative biomasses of plants grown under normal temperature conditions. Wild-type rice plants grown at high temperatures showed a decrease in plant height (FIG. 9C), vegetative biomass and leaf sheath length. Transgenic rice plants overexpressing OsMYB55 grown under high temperature conditions showed less reduction in vegetative biomass as compared to their wild-type counterparts (FIG. 9C). Overexpression of OsMYB55 also moderated the negative effects of the high temperature conditions on the height and leaf sheath length of transgenic rice plants overexpressing OsMYB55.

### Example 8

### The Deleterious Effects of Growth at a Continuous High Temperature are More Severe Under Long Daylight Conditions than Normal Daylight Conditions

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for 4, 9, 11 or 17 weeks under normal growth conditions, under normal daylight conditions with high temperature conditions or under long daylight conditions with either normal temperature conditions or high temperature conditions.

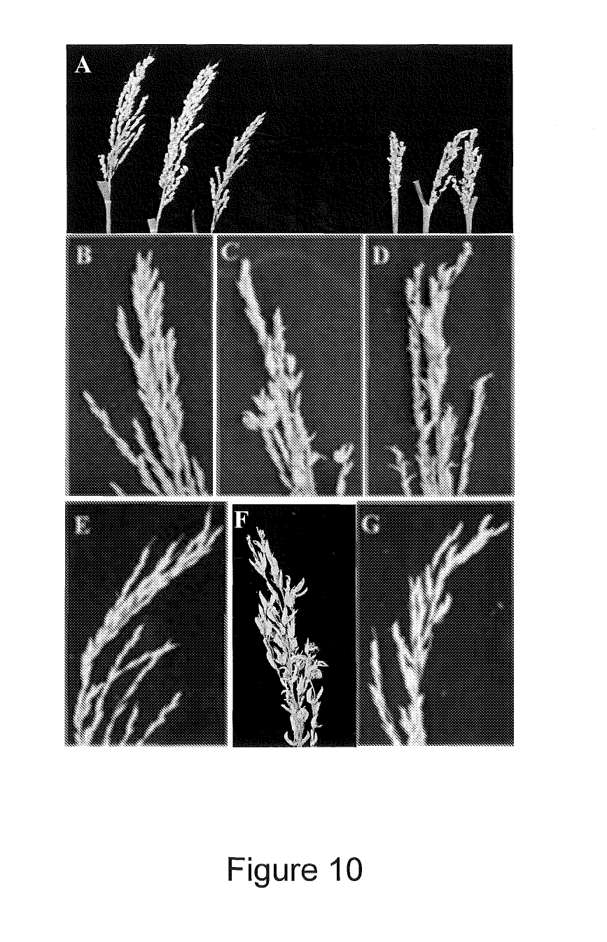

As shown in FIGS. 10A-10G, growth at a continuous high temperature caused deformations of the inflorescences and resulted in complete seed set failure as compared to growth under normal temperature conditions. Overexpression of OsMYB55 did not significantly reduce the occurrence of inflorescence deformations or seed set failure. For both wild-type rice plants and transgenic rice plants overexpressing OsMYB55, the deleterious effects of growth at a continuous high temperature were more severe in plants grown under long daylight conditions than in plants grown under neutral day conditions.

### Example 9

### OsMYB55 Overexpression Improves Plant to High Temperatures

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under normal growth conditions or under normal daylight conditions with high temperature conditions. Following the four-week treatment period, plants were grown under normal growth conditions until harvesting (about 12 weeks).

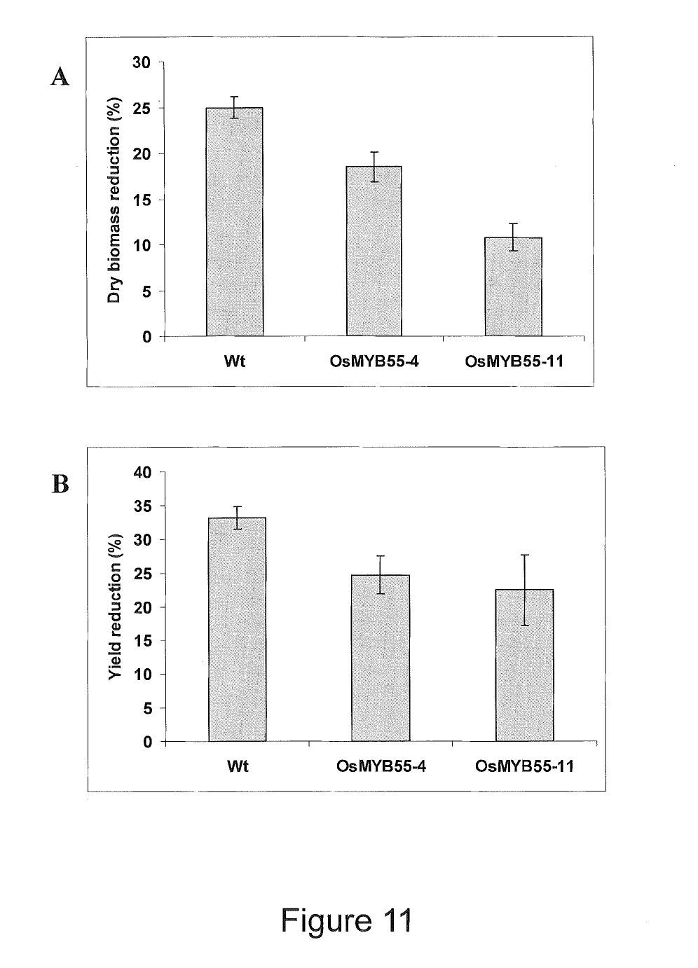

As shown in FIGS. 11A-11B, although growth under high temperature conditions for four weeks resulted in a significant reduction in the total dry biomasses and grain yields of both wild-type rice plants and transgenic rice plants overexpressing OsMYB55, those reductions were less pronounced in transgenic rice plants overexpressing OsMYB55.

### Example 10

### Rice Plants Expressing an OsMYB55-RNAi Construct

An OsMYB55-RNAi construct was prepared according to the methods of Miki et al., PLANT PHYSIOL. 138(4):1903 (2005). cDNA sequence fragments of OsMYB55 491 bp in length and with low similarity to other rice genes were amplified by PCR using the OsMYB55-491 forward primer (5′-CGTCAAGAACTACTGGAACAC C-3′; SEQ ID NO:23) and the OsMYB55-491 reverse primer (5′-CCATGTTCGGGAAGTA GCAC-3′; SEQ ID NO:24). The resultant fragment was cloned into the TOPO® pENTER cloning vector (Life Technologies Corp., Carlsbad, Calif.), and the inverted DNA sequences separated by a GUS intron sequence were generated by the site-specific recombination method in the pANDA binary vector described by Miki et al., PLANT PHYSIOL. 138(4)1903 (2005), downstream of the maize ubiquitin promoter using the Gateway LR Clonase Enzyme Mix (Life Technologies Corp., Carlsbad, Calif.). Transgenic rice lines were obtained using Agrobacterium-mediated transformation, and positively transformed lines were selected according to the methods of Miki et al., PLANT PHYSIOL. 138(4):1903 (2005).

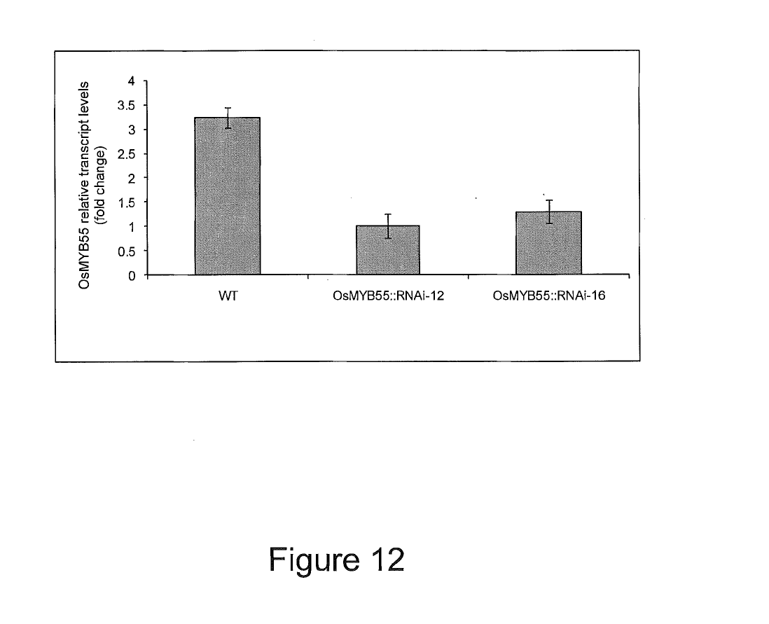

Although the transcript level of OsMYB55 was around three times less in four-week-old rice plants expressing the OsMYB55-RNAi construct (FIG. 12), no phenotypic difference was observed between wild-type rice plants and plants expressing the OsMYB55-RNAi construct. Without wishing to be bound by theory, it is currently believed that the lack of a discernible phenotype in rice plants expressing the OsMYB55-RNAi construct may be due to the large number of members of the R2R3-MYB transcription factor family and the high redundancy among this family.

### Example 11

### OsMYB55 Overexpression Enhances Total Leaf Amino Acid Content

To understand the physiological and molecular mechanisms underlying the enhancement of plant thermotolerance by OsMYB55, various plant tissues were collected and biochemical analyses were carried out to identify differences between wild type rice plants and transgenic rice plants overexpressing OsMYB55 (e.g., differences in sugar content, starch content, hydrogen peroxide content, amino acid content, etc.).

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under normal growth conditions or under normal daylight conditions with high temperature conditions.

Following the four-week treatment period, tissues were collected and freeze dried for 24 hours, then extracted three times using 0.75 mL of 100% methanol. Each extraction was carried out at 70° C. for 15 minutes. Extracts were subjected to chloroform purification by adding 500 μL extract to 355 μL of water and 835 μL chloroform. Following centrifugation, the upper phase was collected and freeze dried, then dissolved in deionized water. Total amino acid content was assayed according to the methods of Rosen, ARCH. BIOCHEM. BIOPHYS. 67:10 (1957).

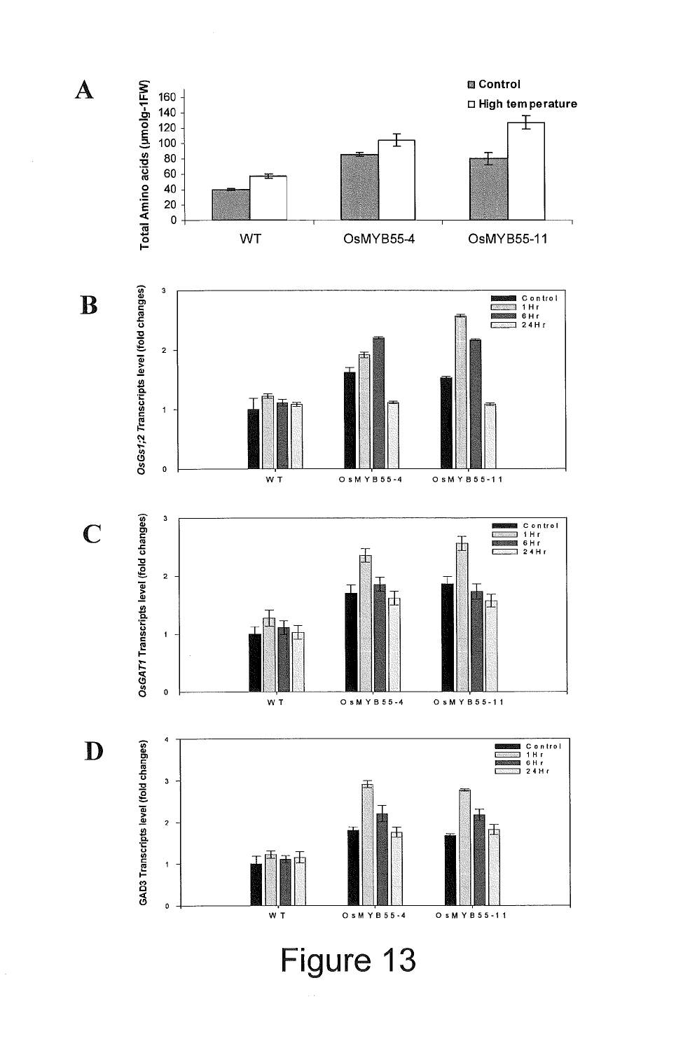

As shown in FIG. 13A, the leaves of transgenic rice plants overexpressing OsMYB55 had a higher total amino acid content than their wild-type counterparts when grown under long daylight conditions with either normal temperature conditions or high temperature conditions. Exposure to high temperature conditions increased the leaf amino acid contents of both wild-type rice plants and transgenic rice plants overexpressing OsMYB55. The leaf amino acid contents of transgenic rice plants overexpressing OsMYB55 were increased more significantly than were the leaf amino acid contents of wild-type rice plants.

### Example 12

### OsMYB55 Overexpression Up-Regulates the Expression of Genes Involved in Amino Acid Metabolism

Based on the total amino acids analysis results, we suspected that OsMYB55 might have a role in the activation of genes involved with amino acid metabolism. To test this hypothesis, a genome-wide transcriptome analysis was conducted using global microarray analysis of wild-type rice plants and transgenic rice plants overexpressing OcMYB55 exposed to high temperatures.

Because microarray analysis can miss more subtle changes in gene expression, quantitative real-time PCR was performed using primers corresponding to other genes involved in amino acids biosynthesis and transport. Quantitative real-time RT-PCR was carried out using specific primers designed from the sequence of the chosen genes. Total RNA was isolated from plant tissues using TRI-Reagent® RNA isolation reagent (Sigma-Aldrich Corp., St. Louis, Mo.). To eliminate any residual genomic DNA, total RNA was treated with RQ1 RNase-free DNase (Promega Corp., Madison, Wis.). cDNA was synthesized from total RNA using the Reverse Transcription System kit (Quanta BioSciences, Inc., Gaithersburg, Md.). Primer Express 2.0 software (Applied Biosystems by Life Technologies Corp., Carlsbad, Calif.) was used to design the primers for the target genes. Relative quantification (RQ) values for each target gene relative to the internal control Actin2 were calculated using the 2CT method described by Livak and Schmittgen, METHODS 25:402 (2001).

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under long daylight conditions with normal temperature conditions for four weeks and then exposed to 45° C. for 0, 1, 6 or 24 hours.

Three candidate genes important for amino acid production were found to be up-regulated: OsGS1;2, OsGAT1 and Os3AD3 (FIGS. 13B-130). GS1;2 is involved in converting glutamine into glutamic acid and represents one of the early steps in amino acids biosynthesis. GAT1, also known as carbamoyl phosphate synthetase, is involved in the first committed step in arginine biosynthesis in prokaryotes and eukaryotes (Holden et al., CURRENT OPIN. STRUCTURAL BIOL. 8:679 (1998)). The GAD genes are involved in converting the L-glutamic acid into GABA (Hiroshi, J. MOL. CATALYSIS B: ENZYMATIC 10:67 (2000)). Quantitative real-time PCR analysis revealed the up-regulation of the three aforementioned genes one hour after the exposure of rice plants to 45° C. The transcript levels of OsGAT1 and OsGAD3 decreased to nearly basal levels 6 hours after exposure to 45° C. (FIGS. 13C-13D), while OsGS1;2 transcript levels remained significantly increased 6 hours after exposure to 45° C. (FIG. 13B).

No significant difference was detected in the transcripts of others genes involved in amino acid metabolism.

### Example 13

### OsMYB55 Binds to Genes Involved in Amino Acid Metabolism

DNA sequences corresponding to the promoters of the genes identified in Example 12 (OsGS1;2, OsGAT1 and OsGAD3) were analyzed using the PlantCARE database (Flanders Interuniversity Institute for Biotechnology, Zwijnaarde, Belgium; available at http://bioinformatics.psb.ugent.be/webtools/plantcare/html/). The results showed that all three promoters contain a potential binding site for MYB proteins, a CAGTTA motif. The CAGTTA motif, located 1079 bp, 460 bp and 554 bp from the first ATG codon in the OsGS1;2, OsGAT1 and OsGAD3 cDNAs, respectively. Because the CAGTTA motif is a binding box for MYB transcription factors and is therefore a potential binding site for OsMYB55, electrophoretic mobility shift assays (EMSAs) were carried out to determine whether OsMYB55 binds to the CAGTTA-box of QsGS1;2, OsGAT1 or OsGAT3 in vitro.

Recombinant OsMYB55 was prepared as follows. The full-length coding region of OsMYB55 cDNA was amplified using the MYB55-P28-BamHI forward primer (5′-CGCGGAT CCATGGGGCGCGCGCCQT-3′; SEQ ID NO:25) and the MYB55-P28-HinlIl reverse primer (5′-CCCAAGCTTTGTCAGGGTGTTGCAGAGACCCTGT-3′; SEQ ID NO:26). The PCR product and a pET15b vector (Novagen®, EMB Biosciences) were digested with BamHI and HindIII. After ligation, the construct was transformed into Arctic Express (DE3) RIL competent cells (Agilent Technologies, Santa Clara, Calif.) according to the manufacturer's instructions. Recombinant OsMYB55 was purified using a His-tag purification system (Qiagen, Inc., Valencia, Calif.).

The potential OsMYB55 binding sites from the promoters of OsGS1;2, OsGAT1 and OsGAD3 were amplified using specific primers to produce. DNA products containing one copy of their respective MYB binding boxes.

EMSAs were carried out with varying amounts of the recombinant OsMYB55 protein (0, 10, 20 or 40 μg) and the QsGS1;2, OsGAT1 and OsGAD3 DNA products (0 or 200 ng) using an EMSA kit (Cat. # E33075, Molecular Probes, Inc., Eugene, Oreg.). The DNA- and/or protein-containing samples were loaded into a Ready Gel TBE, gradient 4-20% polyacrylamide native gel (Bio-Rad Laboratories, Hercules, Calif.) at 200 V for 45 minutes. The DNA in the gel was stained using the SYBR® Green provided in the EMSA kit and visualized using the ChemiDoc™ imaging system (Bio-Rad Laboratories, Hercules, Calif.).

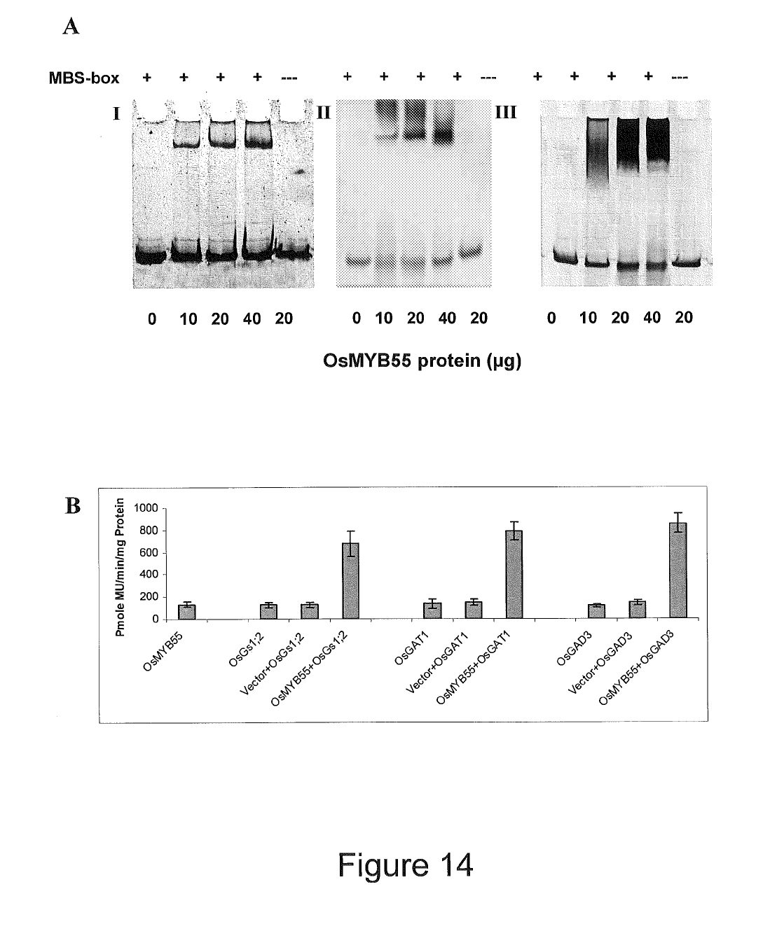

As shown in FIG. 14A, QsMYB55 strongly binds to the QsGS1;2, QsGAT1 and OsGAD3 promoter sequences containing the CAGTTA box motif.

### Example 14

### OsMYB55 Activates Genes Involved in Amino Acid Metabolism

Binding of the OsMYB55 protein to the promoter sequences of OsGs1;2, OsGAT1 and OsGAD3 supports the idea that OsMYB55 might enhance amino acid content through the activation of these genes. To investigate this hypothesis, a transcription activation assay using a transient gene expression strategy was carried out using GUS as a reporter protein.

DNA sequences corresponding to the OsGS1;2, OsGAD3 and OsGAT1 promoters were cloned into an intron-containing GUS reporter vector. The DNA sequence of the OsGS1;2, OsGAD3 and OsGAT1 promoters (1.5-2 kb upstream of the ATG start codon of the cDNA) was amplified from rice genomic DNA using the OsGS1;2 promoter forward primer (5′-CACCTGCGGTGAATGGAAGACGTTTG-3′; SEQ ID NO:27) and the OsGS1;2 promoter reverse primer (5′-TGCTCAAAGCAGAAGAGATCTGAATGAG-3′; SEQ ID NO:28), the OsGAT1 promoter forward primer (5′-CACCGACGGAGGAAGTAGTGTG GAACCAT-3′; SEQ ID NO:29) and the OsGAT1 promoter reverse primer (5′-TGGTG GTAGGGTG CGGC-3′; SEQ ID NO:30) or the OsGAD3 promoter forward primer (5′-CACCCAGATCAAATGTCA AAAGGGGCG-3′; SEQ ID NO:31) and the OsGAD3 promoter reverse primer (5′-CTTGCCT GCCGAGCTATCAACC-3′; SEQ ID NO:32). The resulting fragments were cloned into the TOPO pENTER vector (Life Technologies Corp., Carlsbad, Calif.), and the final construct was prepared by the site-specific recombination method in the DMC162 gateway vector by Gateway® LR Clonase® enzyme mix (Life Technologies Corp., Carlsbad, Calif.). OsMYB55 was inserted next to the 35S promoter in the DMC32 vector using the OsMYB55-Pent forward primer (5′-ATGGGGCGCGCGCCGTG-3′; SEQ ID NO:33) and the OsMYB55-Pent reverse primer (5′-CTATGTCAGGGTGTTGCAG AGACC-3′; SEQ ID NO:34). This plasmid was used as an activator in the co-transformation transient expression analysis. To normalize the GUS activity values, the firefly (Photinus pyralis) luciferase gene driven by the 35S promoter in the pJD312 plasmid (kindly donated from Dr. Virginia Walbot, Stanford University) was used. Equal amounts of DNA from the different plasmid constructs were transformed by particle bombardment into four-week-old old tobacco (Nicotiana plumbaginifolia) leaves. After incubation for 48 hours at room temperature in the dark, total protein was extracted from each sample and GUS and luciferase activities were measured. GUS activity was determined by measuring the cleavage of β-glucuronidase substrate 4-methylumbelliferyl β-D-glucuronide (MUG). Luciferase activity was measured using the Luciferase Assay System kit (Cat. # E1500, Promega Corp., Madison, Wis.) following the manufacturers' instructions. Empty vectors were used as negative controls in this experiment.

As shown in FIG. 14B, OsMYB55 activated the expression of OsGs1;2, GAT1 and GADS in tobacco epidermal cells by almost eight-fold compared to the control experiment. In conjunction with the results of Example 13, these results indicate that OsMYB55 directly regulates the expression of OsGs1;2, OsGAT1 and OsGAD3.

### Example 15

### OsMYB55 Overexpression Enhances Leaf Glutamic Acid and Arginine Content

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under long daylight conditions with either normal temperatures conditions or high temperature conditions.

Following the four-week treatment period, tissues were collected and freeze dried for 24 hours, then extracted three times using 0.75 mL of 100% methanol. Each extraction was carried out at 70° C. for 15 minutes. Extracts were subjected to chloroform purification by adding 500 μL extract to 355 μL of water and 835 μL chloroform. Following centrifugation, the upper phase was collected and freeze dried, then dissolved in deionized water. Glutamic acid and arginine content were determined using L-Glutamic acid and Arginine kits (Megazyme Intl., Bray, Ireland) according to the manufacturer's instructions.

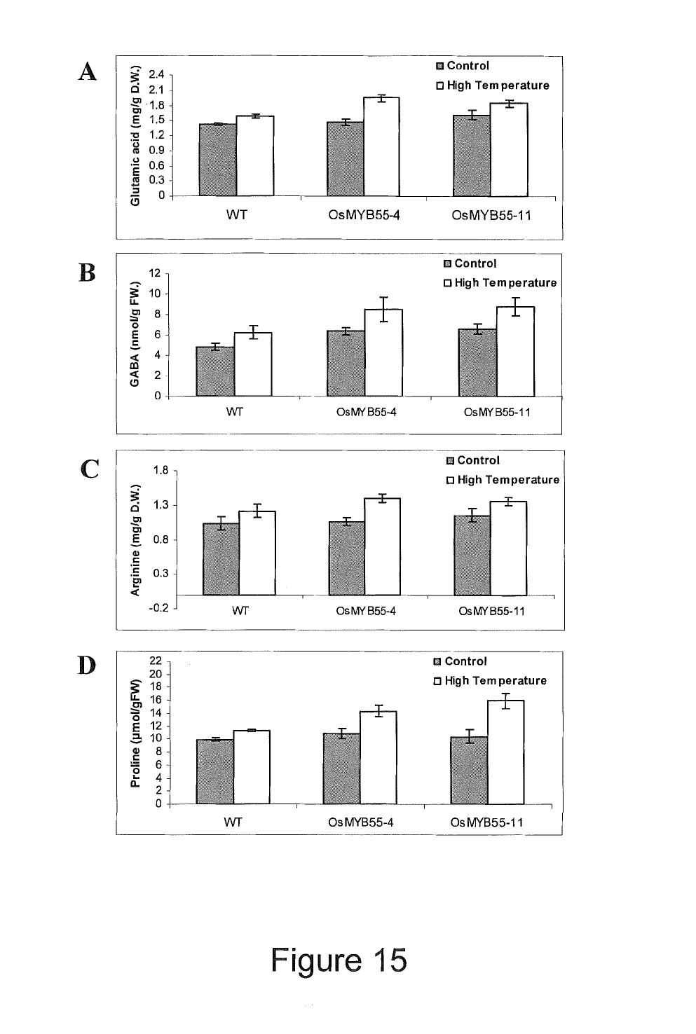

Glutamic acid is one of the first amino acids to be synthesized from nitrogen compounds and can be converted into other amino acids. Consistent with the finding that transgenic rice plants overexpressing OsMYB55 have higher OsGS1; 2 transcript levels than wild-type rice plants when grown under normal growth condition, transgenic rice plants overexpressing OsMYB55 had a higher root glutamic acid content than wild-type rice plants when grown under normal growth conditions. The glutamic acid content of the leaves of transgenic rice plants overexpressing OsMYB55 was similar to that of wild-type rice plants under normal growth conditions (FIG. 15A). Growing the plants under high temperature conditions increased the glutamic acid content of the leaves and leaf sheathes of both wild-type rice plants and transgenic rice plants overexpressing OsMYB55, but the increase was significantly higher in the transgenic rice plants overexpressing OsMYB55 as compared to their wild-type counterparts (FIG. 15A).

Arginine is required for polyamine biosynthesis in plants, which has been reported to be involved in several plant development and stress conditions, including high temperature (Alcazar et al., BIOTECH. LETT. 28:1867 (2006); Cheng et al., J. INTEGRATIVE PLANT BIOL. 51:489 (2009)). Our results showed that leaves of transgenic rice plants overexpressing OsMYB55 had the same level of arginine as those of wild-type rice plants when grown under normal temperature conditions for four weeks (FIG. 15C). Growing the plants under high temperature conditions increased the arginine content of the leaves of both wild-type rice plants and transgenic rice plants overexpressing OsMYB55, but the increase was significantly higher in the transgenic rice plants overexpressing OsMYB55 (FIG. 15C).

### Example 16

### OsMYB55 Overexpression Enhances Leaf GABA Content

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under long daylight conditions with either normal temperatures conditions or high temperature conditions.

Plant tissues were collected and GABA content was determined as described by Zhang and Bown, PLANT J. 44:361 (2005). Briefly, 0.1 g of frozen tissue was extracted with 400 μl methanol at 25° C. for 10 minutes. The samples were vacuum dried, and dissolved in 1 ml of 70 mM lanthanum chloride. The samples were then shaken for 15 minutes, centrifuged at 13,000×g for 5 minutes, and 0.8 ml of the supernatant removed to a second 1.5 ml tube. To this was added 160 μl of 1 M KOH, followed by shaking for 5 min, and centrifugation as before. The resulting supernatant was used in the spectrophotometric GABA assay described below.

The 1 ml assay contained 550 μl of a sample, 150 μl 4 mM NADP+, 200 μl 0.5 M K+pyrophosphate buffer (prepared by adding 0.15 M phosphoric acid drop-wise to reach the pH 8.6), 50 μl of 2 units GABASE per ml and 50 μl of 20 mM α-ketoglutarate. The initial A was read at 340 nm before adding α-ketoglutarate, and the final A was read after 60 min. The difference in A values was used to construct a calibration graph. The commercial GABASE enzyme preparation was dissolved in 0.1 M K-Pi buffer (pH 7.2) containing 12.5% glycerol and 5 mM 2-mercaptoethanol. The resulting solution was frozen until use.

In this study, under normal temperature conditions, we found an increase in the leaf GABA content of the transgenic rice plants overexpressing OsMYB55 compared to their wild-type counterparts (FIG. 15B). Growing the plants under high temperature conditions increased the GABA content of the leaves of both wild-type rice plants and transgenic rice plants overexpressing OsMYB55, but the increase was significantly more obvious in the transgenic rice plants overexpressing OsMYB55 (FIG. 15B).

### Example 17

### OsMYB55 Overexpression Enhances Leaf Proline Content

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under long daylight conditions with either normal temperatures conditions or high temperature conditions.

Plant tissues were collected and proline content was determined according to the protocol previously reported by Abraham et al., METHODS MQL. BIOL. 639:317 (2010). Briefly, 100 mg frozen tissues were extracted by 500 μL 3% sulfosalicylic acid and the supernatant was used for proline quantification. A reaction mixture of 200 μL of glacial acetic acid and 200 μL acidic ninhydrin was added to 200 μL of extract. The reaction was incubated at 96° C. for 60 minutes and terminated in ice. Proline was extracted from the samples in 1 mL toluene and the absorbance in the upper phase was measured at 520 nm after centrifugation. Proline concentration was determined using a standard curve and calculated on fresh weigh basis.

In this study, no significant difference was found in proline content between wild-type rice plants and transgenic rice plants overexpressing OsMYB55 under normal temperature conditions (FIG. 15D). Growing the plants under high temperature conditions increased the proline content of the leaves of both wild-type rice plants and transgenic rice plants overexpressing OsMYB55, but the increase was significantly higher in the transgenic rice plants overexpressing OsMYB55 (FIG. 15D).

### Example 18

### Microarray Hybridization and Data Analysis

Seeds from wild-type rice plants and transgenic rice plants overexpressing OsMYB55 were planted in 590 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions for four weeks, and then exposed to 45° C. for one hour. Leaves of the wild-type and transgenic rice plants were harvested, and total RNA was isolated.

Double-stranded cDNAs were synthesized from 5 μg of total RNA from each sample. Labeled complementary RNA, synthesized from the cDNA was hybridized to a rice whole genome array (Cat. No. 900601, Affymetrix, Inc., Santa Clara, Calif.). The hybridization signal of the arrays was obtained by the GeneChip Scanner 3000 (Affymetrix, Inc., Santa Clara, Calif.) and quantified by Microarray Suite 5.0 (Affymetrix, Inc., Santa Clara, Calif.). The probe set 25 measurement was summarized as a value of weighted average of all probes in a set, subtracting the bottom 5% of average intensity of the entire array using a custom algorithm. The overall intensity of all probe sets of each array was further scaled to a target intensity of 100 to enable direct comparison. Data was analyzed using GeneSpring software (Agilent Technologies, Santa Clara, Calif.). Genes with two-fold change were identified first, and then ANOVA was used to identify significant genes (Welch t-test p-value cutoff at 0.05).

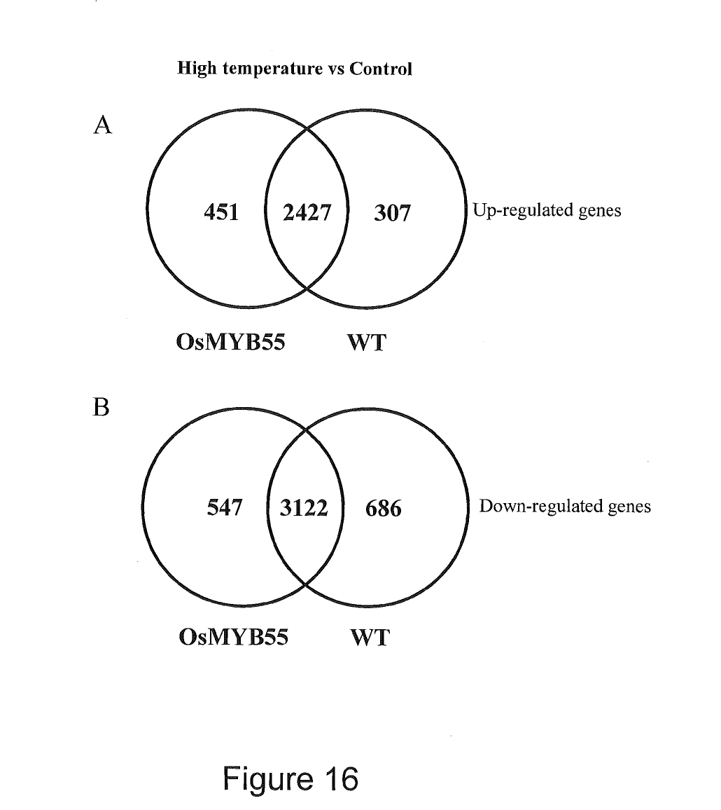

As shown in FIGS. 16A-16B, numerous genes were found to be up-regulated and/or down-regulated in response to high growth temperatures, both in wild-type rice plants and in transgenic rice plants overexpressing OsMYB55.

### Example 19

### Statistical Analyses

All statistical analyses were performed using SigmaStat (SPSS Inc., Chicago, Ill.) with an error rate set at α=0.05. The significance difference between treatments was tested using Tukey's Honestly Significant Difference Test.

### Example 20

### Discussion

We identified a MYB transcription factor that enhances rice plant tolerance to high temperature during the vegetative growth stage. The overexpression of OsMYB55 improved plant growth and productivity under high temperature conditions. The transgenic plants maintain higher plant height and more dry-biomass as compared with the wild-type plants grown under high temperature. Exposure of the wild-type plants for four weeks in the first six weeks of the life cycle to high temperature decreased grain yield at harvest. However, this reduction was significantly less in the transgenic plants. Together, these results indicate that the transgenic lines grow and perform better under high temperature than wild-type. Although, there was a positive effect of OsMYB55 overexpression in plant heat tolerance, a RNAi knockdown of its expression did not show any significant difference from wild-type. This is likely due to the high level of redundancy in this gene family, although it could be due to the fact that these lines still had some expression, albeit lower, of the OsMYB55 gene.

To explore the function of the OsMYB55 in enhancing plant growth under high temperature, different biochemical and molecular analyses have been carried out. Histological analysis of the leaf, leaf sheath and stem tissues of the wild-type and transgenic plants growing at high temperature did not reveal any significant difference (data not shown), suggesting that the higher plant height and leaf sheath length are results of enhancing general plant performance rather than affecting cell expansion or division. In addition, biochemical analysis of starch, sugars and hydrogen peroxide as an indicator for antioxidant activity did not show significant changes in the transgenic plants compared to the wild-type plants (data not shown). Rivero et al., PLANT BIOL. (STUTTG) 6:702 (2004), suggested the importance of nitrogenous compounds and proline in plant heat tolerance. We investigated nitrate and quaternary ammonium compounds in the wild-type and transgenic plants growing at two different temperatures and no significant difference was found (data not shown).

In the present study, plants that overexpressed OsMYB55 had an increase in total amino acid content. Transcript analysis of several Heat Shock Transcription Factors and Heat Shock Proteins known to be involved in plant responses to high temperature revealed no significant difference between the wild-type and the transgenic plants (data not shown). Global transcriptome analysis did result in the identification of three targets for the rice OsMYB55, namely OsGS1;2, GAT1 and GAD3. OsMYB55 binds in vitro to the promoters of these genes and transactivates them in tobacco leaf cells.

OsMYB55 overexpression leads to improved heat tolerance and enhances the level of both total amino acids and glutamic acid, proline, arginine, and GABA in particular. It should be noted that while proline content was increased in the overexpression lines in response to high temperature, there were no significant differences in the expression of the genes involved in proline biosynthesis. These results suggest that the increase in proline content is indirect and could be due to other pathways including protein breakdown as suggested by Becker and Fock, PHOTOSYNTHESIS RES. 8:267 (1986).

In conclusion, overexpression of OsMYB55 leads to increased heat tolerance of rice plants during the vegetative stage. This leads to increased biomass and, if the plants are subsequently grown under normal conditions, increased seed yield. This trait will become of increasing importance as crop yields in many important rice growing regions are decreased due to higher temperatures given global warming. Therefore, it is of great importance to explore different crop genetic solutions to ameliorate this problem. Modulating the expression of OsMYB55 either by itself or in combination with other genes is one potential solution.

The foregoing is illustrative of the present invention, and is not to be construed as limiting thereof. The invention is defined by the following claims, with equivalents of the claims to be included therein.

All publications, patent applications, patents, database accession numbers and other references cited herein are incorporated by reference in their entireties for the teachings relevant to the sentence and/or paragraph in which the reference is presented.

1-42. (canceled)

43. A method of increasing expression of a nucleotide sequence of interest in response to high temperature, comprising:
transforming a plant or plant part with an expression cassette comprising the nucleotide sequence of interest operably associated with:
(a) the nucleotide sequence of SEQ ID NO: 1;
(b) a nucleotide sequence comprising at least 500 consecutive nucleotides of the nucleotide sequence of SEQ ID NO: 1;
(c) a nucleotide sequence that hybridizes to the complete complement of the nucleotide sequence of (a) or (b) under stringent conditions comprising a wash stringency of 50% Formamide with 5×Denhardt's solution, 0.5% SDS and 1×SSPE at 42° C.; and
(d) a nucleotide sequence having at least 95% sequence identity to the nucleotide sequence of any of (a) to (c).

44. The method of claim 43, wherein the nucleotide sequence of interest encodes a polypeptide.

45. The method of claim 43, wherein the nucleotide sequence of interest can be transcribed to produce a functional RNA.

46. The method of claim 43, wherein the nucleotide sequence of interest encodes a water stress polypeptide, an abscisic acid receptor, a dehydration protein, a glutamine synthetase 1;2 (GS1;2), a glutamate decarboxylase 3 (GAD3), a class I glutamine amidotransferase (GATT), or any combination thereof.

47. The method of claim 43, wherein the expression cassette further comprises a nucleotide sequence that encodes a selectable marker.

48. A method of increasing heat tolerance in a plant or plant part, comprising:
transforming a plant or plant part with an expression cassette comprising a nucleotide sequence of interest operably associated with:
(a) the nucleotide sequence of SEQ ID NO: 1;
(b) a nucleotide sequence comprising at least 500 consecutive nucleotides of the nucleotide sequence of SEQ ID NO: 1;
(c) a nucleotide sequence that hybridizes to the complete complement of the nucleotide sequence of (a) or (b) under stringent conditions comprising a wash stringency of 50% Formamide with 5×Denhardt's solution, 0.5% SDS and 1×SSPE at 42° C.; and
(d) a nucleotide sequence having at least 95% sequence identity to the nucleotide sequence of any of (a) to (c),
wherein the transformed plant or plant part expresses the nucleotide of interest, and
wherein expression of the nucleotide sequence of interest increases heat tolerance.

49. The method of claim 48, wherein the method further comprises regenerating a transgenic plant from the transformed plant or plant part.

50. The method of claim 48, wherein the method further comprises obtaining a progeny plant derived from the transgenic plant, wherein the progeny plant comprises the nucleotide sequence of interest in its genome.

51. The method of claim 48, wherein the nucleotide sequence of interest encodes a polypeptide.

52. The method of claim 48, wherein the nucleotide sequence of interest can be transcribed to produce a functional RNA.

53. The method of claim 48, wherein the nucleotide sequence of interest encodes a water stress polypeptide, an abscisic acid receptor, a dehydration protein, a glutamine synthetase 1;2 (GS1;2), a glutamate decarboxylase 3 (GAD3), a class I glutamine amidotransferase (GAT1), or any combination thereof.

54. The method of claim 48, wherein the expression cassette further comprises a nucleotide sequence that encodes a selectable marker.

55. The method of claim 48, wherein the method further comprises exposing the plant, plant part or plant cell to high temperature.

56. The method of claim 48, wherein the method further comprises exposing the plant, plant part or plant cell to abscisic acid.

57. The method of claim 48, wherein the method further comprises exposing the plant, plant part or plant cell to methyl jasmonate.

58. The method of claim 48, wherein the plant is a monocot.

59. The method of claim 48, wherein the plant is a dicot.

60. The method of claim 48, wherein the plant is rice, maize, wheat, barley, sorghum, oat, rye, sugar cane, soybean or Arabidopsis.

61. A transgenic plant produced by the method of claim 49.

62. A seed produced from the transgenic plant of claim 61, wherein the seed comprises the nucleotide sequence of interest.
